SEQUENCE LISTING (1 sequence)

SEQ ID NO: 1 - protein, 8 aa, Artificial Sequence
  synthetic
SIINFEKL

(19) United States
(12) Patent Application Publication
(10) Pub. No.: US 2022/0127575 A1
(43) Pub. Date: Apr. 28, 2022

(54) GENETICALLY MODIFIED HEMATOPOIETIC STEM AND PROGENITOR CELLS (HSPCS) AND MESENCHYMAL CELLS AS A PLATFORM TO REDUCE OR PREVENT METASTASIS, TREAT AUTOIMMUNE AND INFLAMMATORY DISORDERS, AND REBALANCE THE IMMUNE MILIEU AND DYSREGULATED NICHES

(71) Applicant: The United States of America,as represented by the Secretary,Department of Health and Human Services, Bethesda, MD (US)

(72) Inventors: Rosandra N. Kaplan, Potomac, MD (US); Sabina A. Kratzmeier, North Bethesda, MD (US); Daniel W. Beury, Laurel, MD (US); Haiying Qin, Potomac, MD (US)

(73) Assignee: The United States of America,as represented by the Secretary,Department of Health and Human Services, Bethesda, MD (US)

(21) Appl. No.: 17/429,391

(22) PCT Filed: Feb. 10, 2020
(86) PCT No.: PCT/US2020/017515

§ 371 Date: Aug. 9, 2021

Related U.S. Application Data
(60) Provisional application No. 62/803,468, filed on Feb. 9, 2019.

(51) Int. Cl. C12N 5/0789; C12N 5/0775

(52) U.S. Cl. CPC C12N 5/0647; C12N 2510/00; C12N 5/0663

(57) ABSTRACT

Provided are compositions comprising genetically modified hematopoietic stem and progenitor cells (HSPCs) and/or genetically modified mesenchymal cells, wherein the cells contain a vector comprising a transgene, as well as methods of producing the genetically modified HSPCs and genetically modified mesenchymal cells, and methods of treating or preventing cancer (e.g., metastasis) and neurodegenerative conditions, autoimmune disorders, and inflammatory disorders.

### CROSS-REFERENCE TO RELATED APPLICATIONS

This patent application claims the benefit of U.S. Provisional Patent Application No. 62/803,468, filed Feb. 9, 2019, which is incorporated by reference.

### SEQUENCE LISTING

Incorporated by reference in its entirety herein is a nucleotide/amino acid sequence listing submitted concurrently herewith.

### BACKGROUND OF THE INVENTION

Metastasis is the primary cause of death in patients with solid tumors. A deeper understanding of the key regulators of this process is needed in order to develop effective therapeutic strategies.

Harnessing the immune system to target and eliminate distant metastatic lesions is a major challenge. Most immunotherapeutic strategies, including CAR-T cell therapy, are limited by immunosuppression in the tumor and pre-metastatic tumor microenvironment.

There is a desire for an effective prophylactic and/or treatment method for metastasis.

### BRIEF SUMMARY OF THE INVENTION

The invention provides a composition comprising (a) genetically modified hematopoietic stem and progenitor cells (HSPCs), (b) genetically modified mesenchymal cells, or (c) both (a) and (b), wherein the cells contain a vector (e.g., lentiviral vector) comprising a transgene. The invention also provides compositions comprising genetically modified bone marrow-derived myeloid cells, such as CXCR4+ myeloid cells.

The invention provides a method for producing genetically modified hematopoietic stem and progenitor cells (HSPCs) comprising obtaining HSPCs from a mammal, and transfecting the HSPCs with a vector (e.g., lentiviral vector) comprising a transgene, thereby producing genetically modified HSPCs.

The invention also provides a method for producing genetically modified mesenchymal stem cells comprising obtaining mesenchymal stem cells from a mammal, and transfecting the mesenchymal stem cells with a viral vector comprising a transgene, thereby producing genetically modified mesenchymal stem cells.

Additionally, the invention provides treatment methods using the genetically modified HSPCs or genetically modified mesenchymal cells, including for treating cancer in a mammal with cancer, reducing tumor growth or reducing or preventing recurrence of tumor in a mammal with cancer, extending survival time of a mammal with cancer, preventing tumor dormancy in a mammal with cancer, reducing or preventing metastasis in a mammal with cancer, treating a neurodegenerative condition, autoimmune disorder, or inflammatory disorder in a mammal, rebalancing dysregulated niches in a mammal, restoring gut function, memory, behavior, hair growth, nail growth, and/or marrow function in a mammal, and reducing or preventing movement disorders, memory dysfunction, confusion, or motility abnormalities in a mammal.

### BRIEF DESCRIPTION OF THE SEVERAL VIEWS OF THE DRAWINGS

FIGS. 1A-IM demonstrate that immune cell populations are dysregulated and upregulate a core immune suppression gene signature in the lung during rhabdomyosarcoma primary tumor growth at a distant site. Mice were inoculated with M3-9-M rhabdomyosarcoma primary tumor orthotopically in the leg. Lungs from naïve and tumor-bearing mice were harvested at various time points (n=8). Flow cytometry analysis of (A) myeloid populations (Myeloid=CD11b⁺, Granulocytes=CD11b⁺Ly6G⁺, Monocytes=CD11b⁺Ly6G⁻Ly6C⁺, Macrophages=CD11b⁺F4/80⁺, Monocytic DC=CD11b⁺CD11c⁺, Conventional DC=CD11b⁺CD11c⁺) and (B) lymphocyte populations (T Cells=CD3⁺, CD8⁺ T cells=CD3⁺CD8⁺, CD4⁺ T Cells=CD3⁺CD4⁺, NK Cells=CD3⁻ NK1.1⁺). All populations are gated on live CD45⁺ cells. Data was analyzed by ordinary one-way ANOVA with Dunnett's multiple comparisons test between the mean of day 0 and each time point. (C) The PD1^{hi}CD44^{int} population, associated with more dysfunctional T cells, and the activated PD1^{int}CD44^{hi} cells. (D) T cells were isolated from naïve non-tumor-bearing or M3-9-M tumor-bearing mice on day 18 post tumor inoculation by magnetic bead negative selection. Cells were pulsed with CellTrace Violet and activated with Mouse T-Activator CD3/CD28 beads at a 1:1 ratio in the presence of 10 ng/mL of IL-7 for 3 days. Cells were analyzed by flow cytometry gating on CD4⁺ or CD8⁺ T cells. (E) A volcano plot of all expressed genes and a heat map of selected differentially-expressed genes, with the immune suppression gene signature in bold. (F) The top 50 genes with the highest fold change increase and decrease in the lungs of tumor-bearing versus naïve mice. Only genes with a p-value <0.05 are shown. These gene signatures (FIG. 1E-F) mark the metastatic process and can serve as a biomarker for metastatic progression and in gauging response to microenvironment targeting therapy. (G) Gene Ontology (GO) analysis of the top 50 genes upregulated in pre-metastatic compared to naïve lungs. Relevant, significantly-changed GO terms associated with pre-metastatic lungs are shown. Arrows indicate >100 fold enrichment. (H) A bar graph of Ingenuity Pathway Analysis of differentially-expressed genes in pre-metastatic compared to naïve lungs. (I) Lungs were harvested on day 15 and processed into single cell suspension for single cell RNA sequencing (n=4). UMAP plots with dotted lines highlighting most notably changed populations, bar graph of cell number per cluster statistically analyzed by the Kolmogorov-Smirnov test, and pie charts of cell populations illustrate changes in immune cell populations in pre-metastatic lungs. (J) Expression levels of relevant genes in each cluster in naïve and pre-metastatic lungs. (K) Immune cell populations are dysregulated in the pre-metastatic liver during pancreatic cancer progression. Similar to findings in the lungs from sarcoma primary tumor bearing mice, the immune environment of the liver shows elevated myeloid cells and decreased antigen presenting cells and lymphocytes in this tissue prior to pancreatic metastatic progression. Flow cytometry analysis of (upper panel) myeloid populations (Myeloid=CD11b⁺, Macrophages=CD11b⁺F4/80⁺, Monocytes=CD11b⁺CD11c⁻ Ly6C⁺ Ly6G⁻, Granulocytes=Ly6G⁺Ly6C⁺, Conventional DC=CD11b⁺CD11c⁺, and Antigen Presenting Cells MHCII+). PD-L1 expression (lower panel) on myeloid cells and lymphocyte populations (T Cells=CD3⁺, CD8⁺ T cells=CD3⁺CD8⁺, CD4⁺ T Cells=CD3⁺CD4⁺, NK Cells=CD3⁻NK1.1⁺). All populations are gated on live CD45⁺ cells. (L-M) Data from normal human bone marrow was queried using the Human Cell Atlas bone marrow single-cell interactive web portal. Gene expression data of select transcripts per cluster in pre-metastatic lungs.

FIGS. 2A-2E demonstrate genetically engineered myeloid cell (GEMy) production and verification. (A) is a schematic of IL-12 lentiviral maps for the production of GEMys. (B) Cells were harvested after 4 days in culture and analyzed by flow cytometry. Top panel: myeloid cell populations. Bottom panel: Thy1.1 expression as readout for transduction efficiency. (C) is a graph demonstrating that cultured myeloid cells can produce IL-12 when transduced by IL-12-encoding lentivirus. Lin− cells were plated in the presence of 50 ng/mL of IL-6, FLT3-L, and SC with various dilutions of IL-12-encoding lentiviral supernatant dilutions or media. Cultures were assessed for IL-12 by ELISA. (D) Demonstrates that vector control myeloid cells and GEMys traffic to various organs. Tissues were assessed for CD45.1+ cells at 24 hours after intravenous injection by flow cytometry. The phenotype of endogenous and transferred myeloid cells is shown below as pie charts. (E) shows that IL12 levels were elevated in the lung after IL-12 GEMy systemic delivery to mice with localized primary sarcoma tumor in the leg.

FIGS. 3A-J demonstrate that IL12-GEMy treatment rescues and activates T cell populations in pre-metastatic lungs. Mice were inoculated with M3-9-M rhabdomyosarcoma primary tumor orthotopically and not treated (n=9) or treated with non-transduced myeloid cells (n=5) or IL12-GEMys (n=10) on days 12, 19, and 26. Lungs were harvested at primary tumor endpoint on day 27 and analyzed by flow cytometry gated on live CD45⁺ cells. (A) The number of T and NK cells in the lungs (T Cells=CD3⁺, CD8⁺ T cells=CD3⁺CD8⁺, CD4⁺ T Cells=CD3⁺CD4⁺, NK Cells=CD3⁻NK1.1⁺). (B) The proportion of CD8⁺ and CD4⁺ T cells expressing PD1 and CD44 in the lungs to distinguish between the activated PD1^{int}CD44^{hi} and the dysfunctional PD1^{hi}CD44^{int} T cell populations. (C) Graphs demonstrating lymphoid populations in particular organs. Non-transduced myeloid cells or GEMys were transferred into M3-9-M tumor-bearing mice on day 12 post tumor inoculation. Tissues were harvested at primary tumor endpoint (day 27) and lymphocyte populations were analyzed by flow cytometry. (D) Immune cell populations in the lung over time after GEMy treatment. M3-9-M tumor-bearing mice were injected with 1.85×10⁶ GEMys intravenously on day 11 and lungs were harvested 1, 3, and 7 days post-GEMy transfer. The number of T cells and NK cells, the levels of T cell activation markers PD1 and CD44, myeloid populations and transferred cells (IL12-GEMys) over time after GEMy treatment are depicted. (E) The expression of key T cell phenotype genes in bulk RNA isolated from the lungs of tumor-bearing mice three days after IL12-GEMy treatment (n=4). Box plots from single cell RNA sequencing data showing gene expression by cluster (n=4 mice per group). Ingenuity pathway analysis of the cytotoxic T cell cluster from single cell RNA sequencing. Splenocytes from (F) OT-I or (G) OT-II mice were co-cultured with non-transduced myeloid cells or IL12-GEMys at various ratios in the presence of 1 μg/mL OVA₂₅₇₋₂₆₄ (SIINFEKL; SEQ ID NO: 1) or OVA₃₂₃₋₃₃₉ peptide, respectively. To generate activated T cells, splenocytes were first cultured in the presence of 1 μg/mL peptide and 50 Units/mL of recombinant IL-2 for 4 days before co-culture with myeloid cells in the presence of 1 ng/mL peptide. Supernatant from co-cultures was collected after 24 hours and IFNγ was quantified by ELISA (n=3 replicates). (H) Myeloid cell populations in the lungs (Myeloid=CD11b⁺, Monocytic DC=CD11b⁺CD11c⁺, Conventional DC=CD11b⁻CD11c⁺, Granulocytes=CD11b⁺Ly6G⁺, Classical Monocytes=CD11b⁺CD43⁺Ly6C⁺, Macrophages=CD11b⁺CD43⁺Ly6C⁺F480⁺). (I) Non-transduced myeloid cells or IL12-GEMys were transferred into M3-9-M tumor-bearing mice on day 12 post tumor inoculation. Tissues were harvested at primary tumor endpoint (day 27) and myeloid populations in the spleen, lymph node, and tumor were analyzed by flow cytometry. (J) CODEX Immunofluorescence staining of lung sections collected at primary tumor endpoint from non-treated and IL12-GEMy-treated mice (n=3). Nuclear, CD11b, and TCRB staining is shown. T cells are indicated with white arrows.

FIGS. 4A-E demonstrate IL12-GEMy treatment reverses core immune suppression gene program in the lung microenvironment and facilitates immune activation. Mice were inoculated with M3-9-M rhabdomyosarcoma primary tumor orthotopically and treated with IL12-GEMys on day 12. Lungs were harvested three days post-treatment. RNA isolated from flash-frozen lungs was sequenced (n=4). (A) Mice were inoculated with M3-9-M rhabdomyosarcoma primary tumor orthotopically and treated with IL12-GEMys on day 12. Lungs were harvested three days post-treatment. RNA isolated from flash-frozen lungs was sequenced (n=4). Expression of selected genes in the lung that are known to be associated with immune activation and immune suppression comparing naïve, non-treated tumor-bearing mice, and IL12-GEMy-treated tumor-bearing mice. Key immunosuppressive genes from the pre-metastatic gene signature are in bold. (B) Ingenuity Pathway Analysis (IPA) of differential gene expression data from the lungs of IL12-GEMy-treated compared to non-treated mice. (C) Mice were inoculated with M3-9-M rhabdomyosarcoma primary tumor orthotopically and treated with IL12-GEMys on day 12. Lungs were harvested three days post-treatment. RNA isolated from flash-frozen lungs was sequenced (n=4). UMAP projection of single cell RNA sequencing data from the lungs of tumor-bearing mice three days after IL12-GEMy injection (n=4). Labelled dotted lines distinguish most notably changed clusters. IPA of the differential gene expression between IL12-GEMy-treated and non-treated pre-metastatic lungs for individual myeloid cell clusters. (D) The top 50 genes upregulated in each myeloid cell population in the lungs of IL-12 GEMy treated compared to non-treated mice from FIG. 4C defining gene signatures of IL12-GEMy efficacy. (E) Expression levels of key genes associated with response to IL-12, antigen processing and presentation, and immune suppression and the pre-metastatic niche are shown on a per-cluster basis for non-treated and IL12-GEMy-treated tumor-bearing mice.

FIGS. 5A-I demonstrate IL12-GEMy treatment delays tumor progression and metastasis in mice. (A) Mice were treated with one dose of 8×10⁶IL12 GEMys or vector control myeloid cells on day 12 after orthotopic injection of 5×10⁵ M3-9-M-ffLuc2-mCherry rhabdomyosarcoma cells in the gastrocnemius muscle. Mice showed decreased primary tumor growth, prolonged survival and decreased metastatic burden. The IL12-GEMy treatment results in a doubling of overall survival. (B-C). Mice were orthotopically inoculated with 5×10⁵ M3-9-M-ffLuc2-mCherry cells and treated with equal numbers of non-transduced myeloid cells, non-transduced myeloid cells cultured in the presence of 10 ng/mL IL-12, or IL12-GEMys on days 12, 19, and 26 post tumor inoculation (4.1×10⁶, 2.8×10⁵, and 5×10⁶ cells at each time point, respectively). (B) Mice were followed for primary tumor growth and survival (n=8). (C) Lungs were harvested on day 27 and assessed for metastasis by bioluminescent imaging ex vivo by IVIS (no treatment: n=12, myeloid: n=9, IL-12 pre-treated: n=9, IL12-GEMy: n=12). Left panel: Average radiance from harvested lungs. Right panel: Pictures of lungs were normalized, visually inspected for bioluminescence, and categorically grouped into high metastasis (presence of bioluminescence) or no/low metastasis (no bioluminescence). Statistical analysis was determined by Fisher's exact test. (D) IL12-GEMys inhibit tumor progression in a dose dependent fashion. Mice were injected with M3-9-M and on day 12, groups of mice were left untreated or treated with 1×10⁶ or 8×10⁶ IL12-GEMys i.v. (labeled “Low IL12-GEMy” and “High IL12-GEMy,” respectively) and followed for primary tumor growth and survival (n=10). Statistics are shown for low dose IL12-GEMy compared to high dose IL12-GEMy (p=0.0003). Comparison of mice treated with high and low dose IL12-GEMy. (E) Mice were orthotopically inoculated with 5×10⁵ M-3-9M-ffLuc2-mCherry cells. On day 10, groups of mice were left untreated or given a single dose of 2 mg cyclophosphamide (Cy) i.p. On day 12, groups of mice were left untreated or treated with 1×10⁶ or 8×10⁶ IL12-GEMys i.v. (labeled “Low IL12-GEMy” and “High IL12-GEMy,” respectively) and followed for primary tumor growth and survival (n=10). Comparison of mice treated with cyclophosphamide (Cy) and high or low IL12-GEMy doses (Cy alone: n=10, low dose IL12-GEMy: n=10, high dose IL12-GEMy: n=9). Statistics are shown for Cy compared to no treatment (p=0.0035), Cy+Low IL12-GEMy compared to Cy (p=0.006), and Cy+High IL12-GEMy compared to Cy (p<0.001). (F) Mice were injected with 5×10⁴ M-3-9M via tail vein and treated with 8×10⁶ IL12-GEMy i.v. 11 days post tumor injection, then followed for survival and metastatic progression by IVIS (no treatment n=10, IL12-GEMy n=8). Quantification is shown on day 20 post tumor inoculation. (G) Mice were orthotopically injected with 5×10⁵ M3-9-M-ffLuc2-mCherry cells, mice were treated with 8×10⁶ IL12-GEMys on day 17, primary tumors were amputated on day 24 and monitored for survival and metastatic progression by IVIS (no treatment: n=15, IL12-GEMy: n=10). (H) Mice were intrasplenically injected with 5×10⁵ KPC177669-ffLuc2-mCherry cell, spleens were resected, and mice were treated with 8×10⁶ IL12-GEMys on day 5. Mice were monitored for survival and tumor growth by IVIS (no treatment: n=11, IL12-GEMy: n=12). Survival data were tested for significance by log-rank test. (I) Mice were injected with 3×10⁴ 4T1 mammary carcinoma cells s.c. in the mammary fat pad and were treated with Cy/Flu on day 10 followed by 5×10⁶ IL12-GEMys on day 12 and monitored for primary tumor growth and survival.

FIG. 6 depicts that the efficacy of GEMy treatment is dependent on CD8+ T cells. M3-9-M tumor-bearing mice were treated with 200 μg of isotype, anti-CD8, or anti-CD4 antibody or 100 μg of anti-NK-1 antibody i.p. on days 9, 11, and 12. GEMys were injected intravenously on day 12. Depletion antibody treatment was continued at 200 μg per dose every 3-5 days for the duration of the experiment. Graphs illustrating survival and tumor growth of mice treated with GEMys and antibody depletion regimens.

FIG. 7A depicts the generation of ovalbumin-expressing M3-9-M. FIG. 7A is a schematic of the ovalbumin gene inserted into the MSCV lentiviral backbone. M3-9-M-ffuc2-mCherry cells were transduced with the lentiviral vector containing ovalbumin. SIINFEKL (SEQ ID NO: 1)-positive cells were sorted by fluorescence-activated cell sorting (FACS) to establish the M3-9-M-ffluc2-mCherry-OVA cell line. Splenocytes from OT-I mice were activated in culture with 1 μg/mL SIINFEKL (SEQ ID NO: 1) peptide and 50 units/mL of recombinant IL-2. M3-9-M-ffluc2-mCherry and M3-9-M-ffluc2-mCherry-OVA cells were plated and allowed to adhere overnight. OT-1 cells were collected on day 5 post-activation and plated onto the tumor cells at the indicated ratios. Supernatant was collected at 24 hours and analyzed by ELISA for IFNγ (bottom right graph). After the supernatant was removed, luciferin was added to the cells and luminescence was recorded as a readout for luciferase-expressing tumor cell abundance. Percent tumor killing was calculated as follows: 100−[(tumor cells+ T cells)/(tumor cells alone)×100] (bottom left graph).

FIG. 7B demonstrates IL12-GEMy treatment is enhanced by combination with tumor-specific CD8⁺ T cells. Mice were injected with M3-9-M-OVA tumors orthotopically. Splenocytes from Rag1^{-/-} OT-I mice were activated with 1 μg/mL SIINFEKL (SEQ ID NO: 1) peptide and 50 Units/mL of recombinant IL-2 and cultured for 4 days. Mice received either 7.4×10⁶ OT-I T cells, 3.5×10⁶ IL12-GEMys, or both intravenously on day 12 (no treatment: n=10, OT-I T cells: n=11, IL12-GEMy: n=11, OT-I T cells+IL12-GEMy: n=11).

FIGS. 8A-D show IL12-GEMys combined with a standard chemotherapy conditioning regimen used in many adoptive cell therapies. (A) Non-tumor bearing mice were treated with 2 mg of cyclophosphamide and 5 mg of fludarabine i.p. and immune cell populations in the blood were analyzed by flow cytometry 48 hours after treatment. (B) Mice were injected with M3-9-M-OVA and treated with 2 mg of cyclophosphamide and 5 mg of fludarabine i.p. on day 8 (abbreviated Cy/Flu in figure). Graphs depict survival and tumor growth over time (no treatment: n=10, Cy/Flu: n=9, Cy/Flu+IL12-GEMy: n=10). (C) Splenocytes from Rag1−/− OT-1 mice were activated with 1 μg/mL SIINFEKL (SEQ ID NO: 1) peptide and 50 units/mL of recombinant IL-2 and cultured for 4 days. Mice received either 7.4×10⁶ OT-I T cells, 3.5×10⁶ GEMys, or both intravenously on day 10. (no treatment: n=10, Cy/Flu: n=9, Cy/Flu+IL12-GEMy: Cy/Flu T cells and IL12-GEMy n=10) (D) 100% of the mice were cured with IL12-GEMy treatment—these mice were then re-challenged with unlabeled M3-9-M cells or their original tumor cell line, M3-9-M-ffLuc2-mCherry-OVA, in the contralateral leg compared to new naïve age-matched control mice (n=5). Survival data were tested for significance by log-rank test.

FIGS. 9A-9D depict the generation of human GEMys. (A) Schematic of human truncated EGFR (tEGFR) IL-12 lentiviral map for the production of human IL12-GEMys. (B) Human CD34 cells were isolated from apheresis of a patient with mobilized stem cells and cultured for 6 days in StemSpan SFEM in the presence of human recombinant SCF, FLT3L, TPO, and/or IL-6. Cells were washed and transduced with lentivirus containing IL-12 at a multiplicity of infection (MOI) of 50 viral particles per cell and supernatant was collected at 24 hours for ELISA. (C) Human CD34 cells were isolated from apheresis of a patient with mobilized stem cells and cultured for 6 days in StemSpan SFEM in the presence of SCF, FLT3L, TPO, and IL-6. Cells were washed and transduced with lentivirus containing IL-12 for 2 days, at which point supernatant was collected for ELISA and cell pellets were frozen. DNA was isolated from the cell pellets and qPCR was performed to determine copy number. (D) Human myeloid cells were acquired from the RO fraction of elutriated apheresis product from a healthy donor. Cells were transduced with lentiviral vector containing IL-12 for 2 days and supernatant was collected for ELISA.

FIGS. 10A-10C correspond to the production of CXCL9, IL12-CXCL2 GEMys, or dual IL12-CXCL9 lentivirus. (A)-(B) are vector maps for the production of CXCL9 GEMys and dual IL12-CXCL9 GEMys, respectively. (C) Mouse GEMys were generated as previously described and transduced with IL-12, CXCL9, or dual IL12-CXCL9 lentivirus for four days. Culture supernatant was collected analyzed for mouse IL-12 and CXCL9 by ELISA.

FIGS. 11A-11C correspond to production of mouse hyaluronidase (HYAL2) and sperm adhesion molecule 1 (SPAM1) expressing genetically engineered mesenchymal cells (GEMesys). (A-B) are vector maps for the production of mouse HYAL2 and SPAM1 expressing GEMesys. (C) Western blot analysis of Hyal2 and Spam1 expression in primary mouse lung mesenchymal stem cells (MSCs) transduced with a control empty vector, mouse Hyal2 overexpression, or mouse Spam1 overexpression lentiviral vector for 48 hours. Cells were washed and cultured for an additional three days, at which point cells were harvested and cell lysates were prepared and analyzed by western blot. Vinculin serves as the protein loading control and arrows indicate the mHyal2 or mSpam1 protein band.

FIG. 12 is a UMAP which is a graphical representation of visualization tool with an algorithm for dimension reduction based on manifold learning techniques and ideas for topological single cell sequencing data allowing for visualization of multibranched cellular trajectories. Each dot represents an individual cell from a single cell suspension of metastatic osteosarcoma. The clustering analysis reveal cells that are closer together are more related than those further apart. A myeloid cell cluster holds gene expression reminiscent of myeloid mediated immune suppression in the pre-metastatic niche gene signature.

### DETAILED DESCRIPTION OF THE INVENTION

The invention provides a modular system of cellular therapy that can be used as a platform to deliver local targeting of tumors and metastatic microenvironment to treat and/or prevent metastasis, limit autoimmunity (e.g., autoimmune disorders and inflammatory disorders), treat niche dysregulation (altered stem cell niches or dysregulated niches such as neurogenic niche or abnormal bone marrow, crypt or bulge niches), and/or treat neurodegeneration (dementia, Alzheimer's disease, amyotrophic lateral sclerosis, Parkinson's disease, and stroke). The system allows for localized delivery of protein of interest (e.g., immunomodulator, enzyme, substrate, receptor, decoy receptor, antibody, suicide gene system, and/or CRISPR or other gene editing technology for modulation of phagocytic and immune suppression function) without the effects of systemic exposure.

In that regard, the invention provides composition comprising (a) genetically modified hematopoietic stem and progenitor cells (HSPCs), (b) genetically modified mesenchymal cells, or (c) both (a) and (b), wherein the cells contain a vector comprising a transgene or multiple transgenes.

The genetically modified HSPCs can by produced by any suitable method. In one embodiment, the HSPCs are obtained from a mammal and transfected with a vector comprising a transgene, thereby producing genetically modified HSPCs. In a particular embodiment, the HSPCs obtained from the mammal express CD34 (i.e., the cells are CD34+). The HSPCs can be from any suitable source in the mammal, including bone marrow and blood (e.g., peripheral blood). In one embodiment, the HSPCs are obtained by performing lineage depletion of bone marrow or blood cells of CD31, CD3, CD19, CD56, and CD11b; in human patients, CD34+ cells are selected.

Although not wishing to be bound by any particular theory, when the genetically modified HSPCs are delivered to the mammal, it is believed that the genetically modified HSPCs will home to pre-, early, and late metastatic sites better than macrophages and dendritic cells that tend to reside in tissue and home to lymph nodes. Then, the genetically modified HSPCs can proliferate and differentiate into myeloid cells (e.g., monocytes and macrophages) once in the specific tissue site.

The method for producing the genetically modified HSPCs can further comprise differentiating the genetically modified HSPCs into myeloid cells to produce genetically engineered myeloid cells (e.g., macrophages and monocytes). Any suitable means for differentiating the genetically modified HSPCs into myeloid cells can be used. For example, media supporting myeloid cell differentiation includes, but is not limited to, StemSpan SFEM II (StemCell Technologies), StemSpan CD34+ Expansion Supplement (StemCell Technologies), and StemSpan Myeloid Expansion Supplement II (StemCell Technologies).

Accordingly, genetically engineered myeloid cells (GEMys) can be generated in vitro prior to administration to the mammal or the genetically modified HSPCs can be delivered to the mammal.

In one embodiment, the genetically modified myeloid cells are genetically modified bone marrow derived CXCR4+ myeloid cells.

Similarly, the genetically modified mesenchymal cells can be produced by any suitable method. In one embodiment, mesenchymal cells are obtained from a mammal and transfected with a vector comprising a transgene, thereby producing genetically modified mesenchymal cells. The mesenchymal cells can be from any suitable source in the mammal, including bone marrow and blood (e.g., peripheral blood). In one embodiment, the mesenchymal cells are obtained by performing lineage depletion of bone marrow or blood cells of CD45, CD31, CD3, CD19, CD56, and CD11b; cells positive for CD51, PDGFRa (CD140), and CD105 cells are selected. Cells grown in hypoxia enhance stem capacity of these cells.

In one embodiment, the mesenchymal cells are mesenchymal stem cells. The method for producing the genetically modified mesenchymal cells can further comprise differentiating the genetically modified mesenchymal stem cells into genetically engineered stromal cells, such as activated pericytes, myoepithelial cells, fibroblasts (myofibroblasts), and vascular smooth muscle cells.

The HSPCs and/or mesenchymal (e.g., mesenchymal cell bone marrow-derived) cell can express high levels of CXCR4 and home to SDF1+ pre-metastatic niches.

Examples of suitable vectors include plasmids (e.g., DNA plasmids), bacterial vectors (e.g., a Listeria or Salmonella vector), yeast vectors, and viral vectors. In one embodiment, the vector is a viral vector, such as retrovirus, poxvirus (e.g., an orthopox (e.g., vaccinia, modified vaccinia Ankara (MVA), Wyeth, NYVAC, TROYVAC, Dry-Vax, or POXVAC-TC), avipox (e.g., fowlpox, pigeonpox, or canarypox, such as ALVAC), raccoon pox, rabbit pox, capripox (e.g., goat pox or sheep pox), leporipox, or suipox (e.g., swinepox), adenovirus, adeno-associated virus, herpes virus, polio virus, alphavirus, baculorvirus, and Sindbis virus. In a specific embodiment, the vector is a lentiviral vector.

Retroviral vectors, including lentiviral vectors, are suitable delivery vehicles for the stable introduction of a variety of genes of interest into the genomic DNA of a broad range of target cells. Without being bound by theory, the ability of retroviral vectors to deliver unrearranged, single copy transgenes into cells makes retroviral vectors well suited for transferring genes into cells. Further, retroviruses enter host cells by the binding of retroviral envelope glycoproteins to specific cell surface receptors on the host cells. Consequently, pseudotyped retroviral vectors in which the encoded native envelope protein is replaced by a heterologous envelope protein that has a different cellular specificity than the native envelope protein (e.g., binds to a different cell-surface receptor as compared to the native envelope protein) also can be used.

There are many retroviruses and examples include: murine leukemia virus (MLV), lentivirus such as human immunodeficiency virus (HIV), equine infectious anaemia virus (EIAV), mouse mammary tumor virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV). Other retroviruses suitable for use include, but are not limited to, Avian Leukosis Virus, Bovine Leukemia Virus, and Mink-Cell Focus-Inducing Virus. The core sequence of the retroviral vectors can be derived from a wide variety of retroviruses, including for example, B, C, and D type retroviruses, as well as spumaviruses and lentiviruses. An example of a retrovirus suitable for use in the compositions and methods disclosed herein, includes, but is not limited to, lentivirus.

One lentivirus is a human immunodeficiency virus (HIV), for example, type 1 or 2 (i.e., HIV-1 or HIV-2). Other lentivirus vectors include sheep Visna/maedi virus, feline immunodeficiency virus (FIV), bovine lentivirus, simian immunodeficiency virus (SIV), an equine infectious anemia virus (EIAV), and a caprine arthritis-encephalitis virus (CAEV).

In addition to the transgene, the vector can include an expression control sequence operatively linked to the transgene's coding sequence, such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons. Suitable promoters include, but are not limited to, a hVMD2 promoter, an SV40 early promoter, RSV promoter, adenovirus major late promoter, human CMV immediate early I promoter, poxvirus promoter, 30K promoter, I3 promoter, sE/L promoter, 7.5K promoter, 40K promoter, C1 promoter, and EF-1α promoter.

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. For example, the nucleic acid encoding the polypeptide can be operably linked to a CMV enhancer/chicken β-actin promoter (also referred to as a “CAG promoter”).

Additionally, the vector can comprise a reporter to identify the transfection/transduction efficiency of the vector. Exemplary reporters include, but are not limited to, EGFR and CD90.1. As described in Example 9, truncated EGFR (tEGFR) can be used as a reporter to measure transduction efficiency and as a potential safety switch to deplete transduced cells in vivo by using anti-EGFR antibody (such as Cetuximab).

A nucleic acid encoding the polypeptide can be cloned or amplified by in vitro methods, such as the polymerase chain reaction (PCR), the ligase chain reaction (LCR), the transcription-based amplification system (TAS), the self-sustained sequence replication system (3SR) and the QP replicase amplification system (QB). For example, a polynucleotide encoding the polypeptide can be isolated by polymerase chain reaction of cDNA using primers based on the DNA sequence of the molecule. A wide variety of cloning and in vitro amplification methodologies are well known to persons skilled in the art.

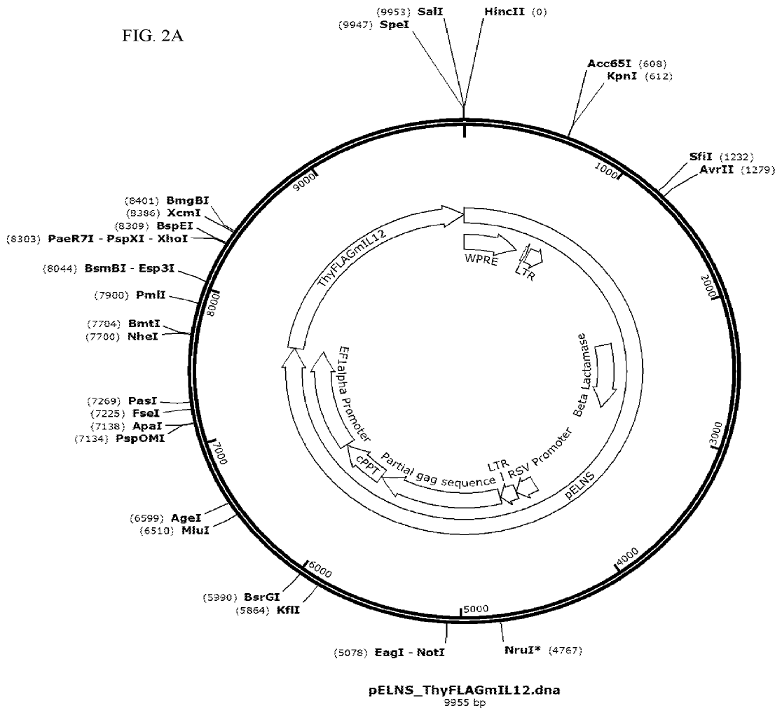

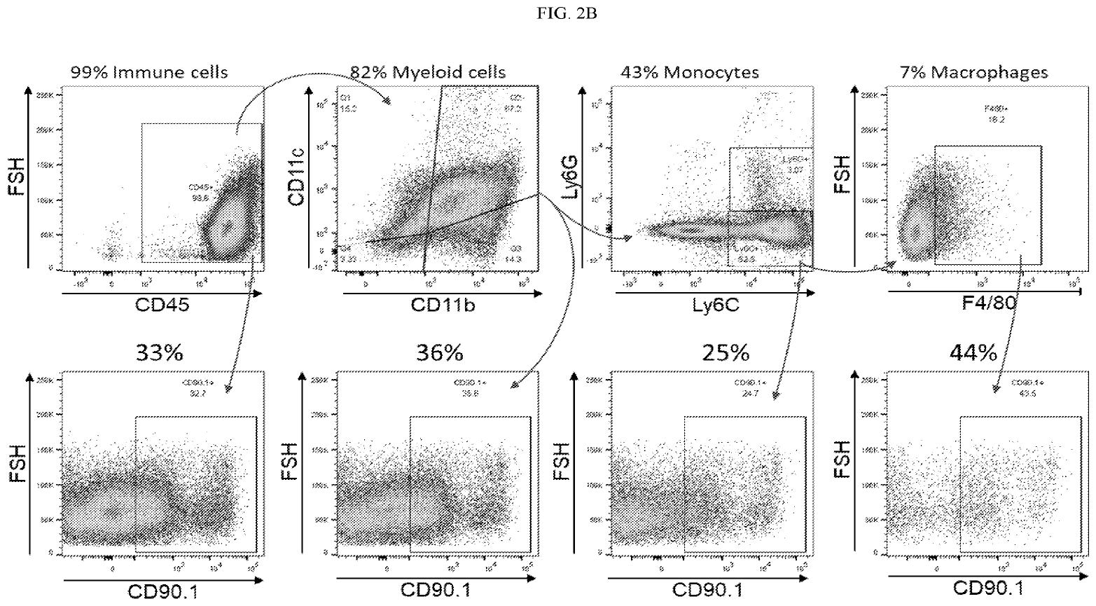

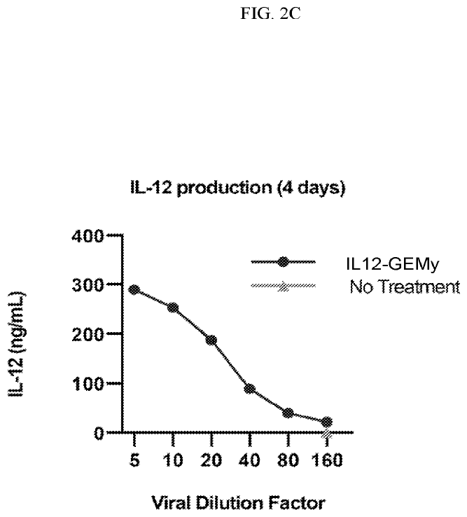

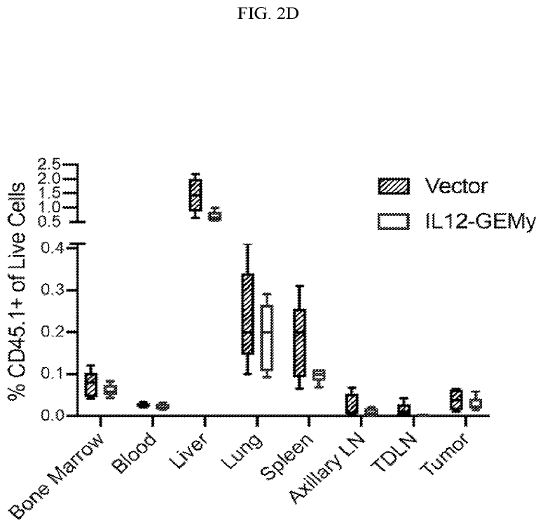

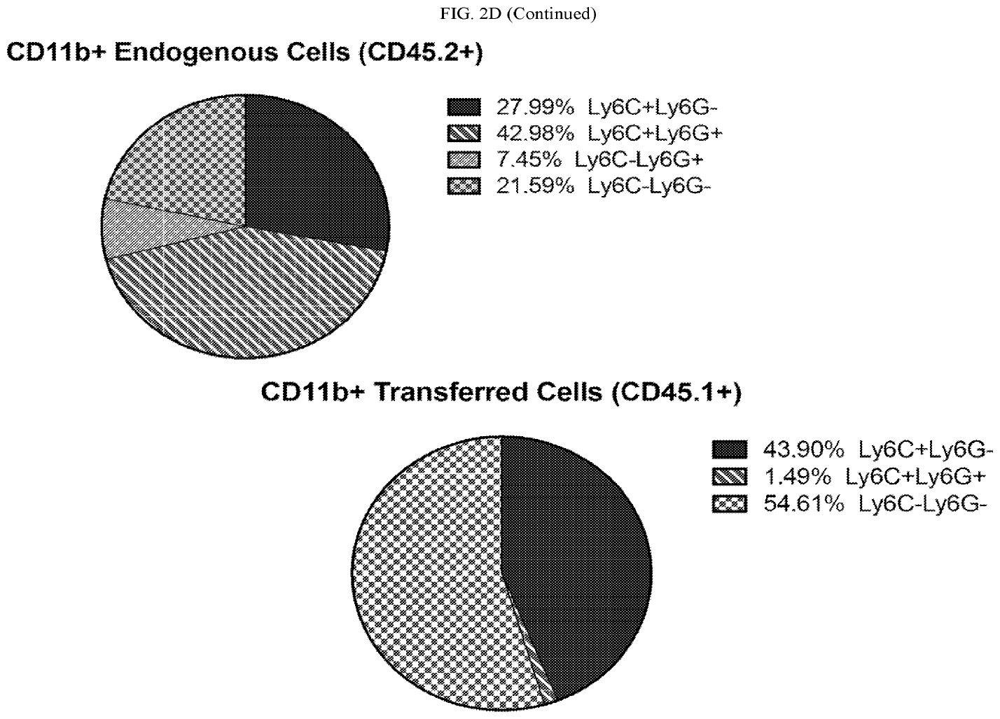

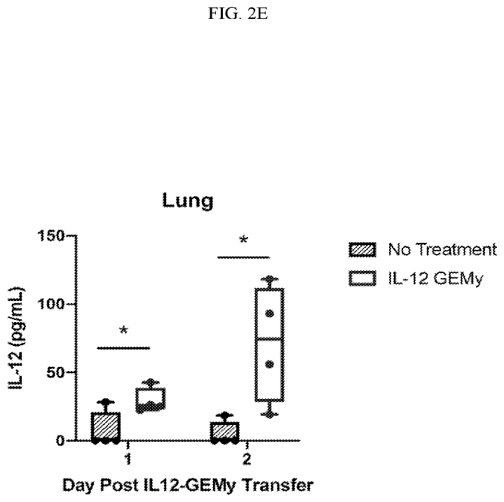

Exemplary vectors for use in the invention include those depicted in FIGS. 2A, 9A, 10A, 10B, 11A, and 11B.

The transgene can be any suitable transgene, such as transgene encoding one or more of a cytokine, a chemokine, an enzyme, a substrate, a receptor decoy/dead receptor (TNFα decoy/dead receptor), an antibody (e.g., scFv, IgG, or a bispecific or trispecific antibody for secretion, binding, and opsonizing tumor/increasing phagocytosis; or an antibody-drug that targets tumor cells, damaged neurons, or damaged, dead or dying cells), a suicide gene system, a CRISPR edited gene, or a protein induced after binding a receptor.

In one embodiment, the transgene encodes an enzyme that is an extracellular matrix remodeling protein, such as hyaluronidase. While not wishing to be bound by any particular theory, the expression of the extracellular matrix remodeling protein alters the extracellular matrix by interacting with stromal cells to limit, prevent, and/or treat metastasis.

In another embodiment, the transgene encodes a suicide gene system, including a Herpes Simplex Virus Thymidine Kinase (HSVTK)/Ganciclovir (GCV) suicide gene system and an inducible Caspase suicide gene system. The suicide gene system kills disseminated tumor cells independent of the host's immune system.

The transgene also can encode a cytokine, chemokine, or a related protein, such as IL-12, CXCL9, CXCL10 (anti-tumor); IL-10, SMAD (immune suppressing to rebalance the immune milieu), TGFβIL-2, TREM1, TREM2, CD2AP, GPR32, FPR2, P2ry2, P2ry6, ChemR23, ERV, GPR32, GPR18, GPR37, and LGR6.

In one embodiment, the method includes the administration of one or more (e.g., 2, 3, 4, or more) transgenes, which may or may not perform complementary functions. For example, IL-12 recruits T-cells and CXCL9 activates T cells for the purpose of treating and/or preventing tumor metastasis. The invention encompasses the co-secretion or co-expression of two different transgenes (bi-GEMy; e.g., IL-12 and CXCL9 as described in Example 10) and co-expression or co-secretion of three transgenes (Tri-GEMy).

The one or more transgenes can be present in a single vector. Alternatively, one or more vectors can be employed each containing one or more transgenes, wherein the transgenes in the one or more vectors can be the same or different.

In one embodiment, the method includes different temporal administrations of one or more complementary transgenes. For example, a first population of genetically modified HSPCs, genetically modified mesenchymal cells, genetically engineered myeloid cells, genetically engineered stromal cells, or compositions thereof that has been transfected with a first vector comprising a first transgene is administered prior to (i.e., sequential delivery) a second population of genetically modified HSPCs, genetically modified mesenchymal cells, genetically engineered myeloid cells, genetically engineered stromal cells, or compositions thereof that has been transfected with a second vector comprising a second transgene, wherein the first and second transgenes encode complementary proteins. In one example, the invention provides temporal delivery of one local protein or decoy receptor or TRAP after binding or secreting another factor.

In another embodiment, a first population of genetically modified HSPCs, genetically modified mesenchymal cells, genetically engineered myeloid cells, genetically engineered stromal cells, or compositions thereof that has been transfected with a first vector comprising a first transgene is administered at the same time as a second population of genetically modified HSPCs, genetically modified mesenchymal cells, genetically engineered myeloid cells, genetically engineered stromal cells, or compositions thereof that has been transfected with a second vector comprising a second transgene, wherein the first and second transgenes encode complementary proteins.

The invention includes bi- or tri-GEMys/GEMesys. For example, a bi-GEMy/GEMesys binds a tumor antigen to induce cytokine release. The invention includes co-administration or sequential administration of mono-, bi- or tri-GEMys/GEMesys.

The invention also provides an inducible system of genetically modified HSPCs, genetically modified mesenchymal cells, genetically engineered myeloid cells, genetically engineered stromal cells, or compositions thereof. For example, in an inducible system, the expression of one or more transgenes from genetically modified HSPCs, genetically modified mesenchymal cells, genetically engineered myeloid cells, genetically engineered stromal cells, or compositions thereof can depend on temperature, pH, and/or the presence of particular drugs. An inducible system can be used to target specific cells or tissues in a mammal and/or target particular disorders.

In one embodiment, the method includes the administration of a transgene encoding a protein that only gets released after exposure to a specific extracellular matrix protein or in response to a tumor specific protein or in response to a particular secreted protein, pH change or oxygen level or receptor expression for example.

The genetically modified HSPCs, genetically modified mesenchymal cells, genetically engineered myeloid cells, genetically engineered stromal cells, or compositions thereof (e.g., containing a pharmaceutically acceptable carrier) can be administered to a mammal with cancer in order to treat cancer.

Non-limiting examples of specific types of cancers include cancer of the head and neck, eye, skin, mouth, throat, esophagus, chest, bone, lung, colon, sigmoid, rectum, stomach, prostate, breast, ovaries, kidney, liver, pancreas, brain, intestine, heart or adrenals. More particularly, cancers include solid tumor, sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendothelio sarcoma, synovial sarcoma, medullary thyroid carcinoma, adrenocortical carcinoma, desmoplastic small round cell tumor (DSRCT), malignant peripheral nerve sheath tumors (MPNST), pericytoma, NTRK+ and NTRK− fusion tumors, rhabdoid tumors, Fusion negative, Ewings like sarcomas, mesothelioma, Ewings tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma, retinoblastoma, a blood-bom tumor, acute lymphoblastic leukemia, acute lymphoblastic B-cell leukemia, acute lymphoblastic T-cell leukemia, acute myeloblastic leukemia, acute promyelocytic leukemia, acute monoblastic leukemia, acute erythroleukemic leukemia, acute megakaryoblastic leukemia, acute myelomonocytic leukemia, acute lymphocyctic leukemia, acute undifferentiated leukemia, chronic myelocytic leukemia, chronic lymphocytic leukemia, hairy cell leukemia, or multiple myeloma as well as ultra/very rare cancers such as globus tumors, PECOMAs, IMF, GIST, chordomas, etc.

Treatment of cancer comprises, but is not limited to, destroying tumor cells, reducing tumor burden, inhibiting tumor growth, reducing the size of the primary tumor, reducing the number of metastatic lesions, increasing survival of the individual, delaying, inhibiting, arresting or preventing the onset or development of metastatic cancer (such as by delaying, inhibiting, arresting or preventing the onset of development of tumor migration and/or tumor invasion of tissues outside of primary cancer and/or other processes associated with metastatic progression of cancer), delaying or arresting primary cancer progression, improving immune responses against the tumor, improving long term memory immune responses against the tumor antigens, and/or improving the general health of the patient with illness. It will be appreciated that tumor cell death can occur without a substantial decrease in tumor size due to, for instance, the presence of supporting cells, vascularization, fibrous matrices, etc. Accordingly, while reduction in tumor size is preferred, it is not required in the treatment of cancer.

Administration of the genetically modified HSPCs, the genetically modified mesenchymal cells, genetically engineered myeloid cells, genetically engineered stromal cells, or compositions thereof can be “prophylactic” or “therapeutic.” When provided prophylactically, the genetically modified HSPCs, genetically modified mesenchymal cells, genetically engineered myeloid cells, genetically engineered stromal cells, or compositions thereof can be administered to a mammal, with the goal of preventing, inhibiting, or delaying metastases of tumors and/or generally preventing or inhibiting progression of cancer in an individual, and generally to allow or improve the ability of the host's immune system to fight against a tumor that the host is susceptible of developing. When provided therapeutically, the genetically modified mesenchymal cells, genetically engineered myeloid cells, genetically engineered stromal cells, or compositions thereof are provided at or after the diagnosis of the cancer, with the goal of ameliorating the cancer, such as by reducing tumor burden in the individual; inhibiting tumor growth in the individual; increasing survival of the individual; and/or preventing, inhibiting, reversing or delaying progression of the cancer in the individual.

Accordingly, the invention provides methods of reducing tumor growth or reducing or preventing recurrence of tumor growth in a mammal with cancer, extending survival time of a mammal with cancer, and preventing tumor dormancy in a mammal with cancer by administering the genetically modified mesenchymal cells, genetically engineered myeloid cells, genetically engineered stromal cells, or compositions thereof to the mammal. In one embodiment, the cancer or tumor has not yet metastasized in the mammal.

Additionally, the invention provides a method of reducing or preventing metastasis in a mammal with cancer comprising administering the genetically modified mesenchymal cells, genetically engineered myeloid cells, genetically engineered stromal cells, or compositions thereof to the mammal.

When the mammal has already been diagnosed with cancer (e.g., metastatic cancer), the genetically modified HSPCs, the genetically modified mesenchymal cells, genetically engineered myeloid cells, genetically engineered stromal cells, or compositions thereof can be administered in conjunction with other therapeutic treatments such as chemotherapy, surgical resection of a tumor, treatment with targeted cancer therapy, allogeneic or autologous stem cell transplantation, T cell adoptive transfer, other immunotherapies, and/or radiation. In particular, the genetically modified HSPCs, the genetically modified mesenchymal cells, genetically engineered myeloid cells, genetically engineered stromal cells, or compositions thereof can be administered (concurrently or concomitantly) with an additional therapeutic agent including, but not limited to, chimeric antigen receptor (CAR)-modified T cells, T cell receptor (TCR)-modified T cells, a dendritic cell vaccine, an oncolytic virus, chemotherapy, a small molecule, a monoclonal antibody or antigen binding fragments thereof, hormone-blocking therapy, and/or radiation therapy.

Most T cell therapies currently used in the clinic are given following a pre-conditioning regimen of cyclophosphamide and fludarabine (Cy/Flu). Therefore, the method of the invention can include the administration of cyclophosphamide, fludarabine, or a combination thereof.

In one embodiment, the method of treating cancer includes surgical resection of a tumor and administration of the genetically modified HSPCs, the genetically modified mesenchymal cells, genetically engineered myeloid cells, genetically engineered stromal cells, or compositions thereof. While not wishing to be bound by any particular theory, it is believed that administration of the genetically modified HSPCs will result in the genetically modified HSPCs homing to distant tissue sites and prevent or limit metastatic outgrowth.

The invention also provides a method of treating a neurodegenerative condition, autoimmune disorder, or inflammatory disorder in a mammal comprising administering the genetically modified HSPCs, the genetically modified mesenchymal cells, genetically engineered myeloid cells, genetically engineered stromal cells, or compositions thereof. Exemplary neurodegenerative conditions, autoimmune disorders, and inflammatory disorders include, but are not limited to, Alzheimer's disease, amyotrophic lateral sclerosis, inflammatory bowel disease (IBD), rheumatoid arthritis, graft versus host disease (GVHD), multiple sclerosis, and alopecia areata.

Additionally, the invention provides a method of rebalancing dysregulated niches; restoring gut function, memory, behavior, hair growth, nail growth, and/or marrow function; or reducing or preventing movement disorders, memory dysfunction, confusion, or motility abnormalities in a mammal comprising administering the genetically modified HSPCs, the genetically modified mesenchymal cells, genetically engineered myeloid cells, genetically engineered stromal cells, or compositions thereof.

The genetically modified HSPCs, genetically modified mesenchymal cells, genetically engineered myeloid cells, genetically engineered stromal cells, or compositions thereof can be administered to a mammal by various routes including, but not limited to, subcutaneous, intramuscular, intradermal, intraperitoneal, intrathecal, intravenous, and intratumoral. In one embodiment, the genetically modified mesenchymal cells, genetically engineered myeloid cells, genetically engineered stromal cells, or compositions thereof can be directly administered (e.g., locally administered) by direct injection into the cancerous lesion or tumor. When multiple administrations are given, the administrations can be at one or more sites in a host and a single dose can be administered by dividing the single dose into equal portions for administration at one, two, three, four or more sites on the individual.

The following formulations are merely exemplary and are in no way limiting. Suitable formulations include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, com, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets.

Preferably, the cells are administered by injection, e.g., intravenously. The pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, Ill.), PLASMA-LYTE A (Baxter, Deerfield, Ill.), about 5% dextrose in water, or Ringer's lactate. In an embodiment, the pharmaceutically acceptable carrier is supplemented with human serum albumen.

The following examples further illustrate the invention but, of course, should not be construed as in any way limiting its scope.

### EXAMPLES

Metastasis is a very demanding process even for the most fit of tumor cells. Acquiring all the genetic acumen needed to successfully proliferate, survive, develop resistance to chemotherapy, migrate, invade, and colonize does not impart disseminated tumor cells with guaranteed success to form metastasis in distant organ sites. Despite these challenges, metastasis does occur, suggesting that survival of these cells is regulated by stromal and immune cell populations. The major events allowing for successful metastasis, include the formation of a metastatic niche environment that supports disseminated tumor cell adhesion, growth, and survival in distant tissue sites (see Kaplan et al., Nature, 438(7069): 820-827 (2005); and Murgai et al., Nat. Med., 23(10): 1176-1190 (2017)).

To explore the impact of reprogramming myeloid cells from immune suppressive to immune activating in order to reverse the functional loss in adaptive cell immunity, genetically-engineered myeloid cells (GEMys) that are engineered to secrete IL-12 were adoptively transferred into mice to determine their homing and functional capacity to rebalance the immune microenvironment in the tumor and early metastatic sites and promote robust anti-tumor immunity. This restoration of immune balance is associated with a strong impact on metastatic outcome, with tumor-bearing mice treated with GEMys surviving twice as long as tumor-bearing mice treated with unaltered or vector control myeloid cells.

The materials and methods for the following examples are set forth below.

Synthesis of Human GEMys

Apheresis product from healthy donors (NIH Bloodbank) was enriched for CD34+ cells using a magnetic bead-based CD34+ cell isolation kit (Miltenyi Biotec) as per the recommended manufacturer's protocol. The isolated cells were cultured in StemSpan SFEM II (StemCell Technologies) supplemented with 100 units/mL penicillin, 100 μg/mL streptomycin, and StemSpan CD34+ Expansion Supplement (StemCell Technologies), StemSpan Myeloid Expansion Supplement II (StemCell Technologies), or cytokines such as SCF, FLT3L, IL-6, and TPO (BioTechne) at 37° C. for 96 hours. Cells were transduced between days 2-6 of culture with lentivirus encoding human IL-12 with truncated EGFR as a reporter for transduction efficiency. Transduction was confirmed by performing an ELISA for IL-12 on the culture supernatant, quantitative PCR, and by staining the putative GEMys for truncated EGFR and analysis by flow cytometry.

Synthesis of Mouse GEMys

Bone marrow was flushed from the femurs and tibia of syngeneic mice and enriched for hematopoietic stem and progenitor cells using a magnetic bead-based lineage depletion kit (StemCell Technologies) as per the recommended manufacturer's protocol. The isolated cells were cultured in StemSpan SFEM (StemCell Technologies) supplemented with 50 ng/mL of mouse SCF, IL-6, and FLT3-L (Bio-Techne), 100 units/mL penicillin, and 100 μg/mL streptomycin at 37° C. for 96 hours. Cells were transduced during the start of culture with lentivirus encoding either mouse IL-12 and/or CXCL9 and with CD90.1 as a reporter for transduction efficiency. Transduction efficiency was confirmed by performing an ELISA for IL-12 on the culture supernatant and by staining the putative GEMys for CD90.1 and analysis by flow cytometry.

Human and Mouse GEMesys (Genetically Engineered Mesenchymal Cells)

(1) Media Preparation (MesenPure Media (MSC+ Media))

Thaw 10× Supplement at room temperature or at 4° C. overnight. Mix thoroughly. Once thawed, use immediately or aliquot and store at −20° C. After thawing aliquots, use immediately. Do not re-freeze.

Add 50 ml of 10× Supplements to 450 ml of Basal Medium. Add 5 ml of Pen/Strep/Glutamine. Mix thoroughly.

Thaw MesenPure at room temperature. Mix thoroughly. Once thawed, use immediately or aliquot and store at −20° C. After thawing aliquots, use immediately. Do not re-freeze.

Dilute MesenPure 1:1000 in complete MesenCult expansion media (i.e. add lul MesenPure per 1 ml complete medium) and mix thoroughly. If not used immediately, store media at 4° C. for up to 2 weeks (can be longer, e.g., ˜2 months).

(2) Isolation from Bone:

Carefully pick meat off the bones (such as 2 tibiae and 2 femurs). Add bones to a mortar and pestle+5 mL of sterile collection media (HBSS, 500 ml; FPS, 10 ml). Crush bones until no more large chunks of bone and marrow remain. Use serological pipette to gently break up clumps and apply to 70 um filter over a 50 mL conical tube. Wash remaining bone pieces in mortar and pestle with 5 ml collection media and apply to filter. Centrifuge cells at 1400 rpm for 4 minutes. Decant the supernatant and resuspend the pellet in 10 mL of collection media. If clumpy, filter again into new 50 ml conical tube. Centrifuge cells at 1400 rpm for 4 minutes. Decant the supernatant and resuspend the pellet in 2 mL of MesenPure media (MSC+ media).

Plate 1 ml of cell suspension into 2-10 cm plates and add 6 ml MesenPure media (MSC+ media) to each plate. Place in hypoxia (5% 02) incubator. After 3 hours of incubation, aspirate media and gently wash plate with PBS (repeat wash). Add 6 ml MesenPure media (MSC+ media) to plate and place in hypoxia (5% 02) incubator.

(3) Isolation from Soft Tissues (Liver, Lung):

Chop organ up into small pieces using forceps and scalpel. Place tissue into 1.5 ml Eppendorf tube containing 1 ml collagenase digestion media (HBSS, 10 ml; Collagenase 1, 10 mg; DNAase 1, 100 μL; Dispase 2, 100 μL). Incubate for 20 minutes on the shaker at 37° C.

After incubation, transfer digested tissue to 70 um filter over a 50 ml conical tube. While adding 5 ml sterile collection media with serological pipet, smash tissue using plunger. Use an extra 5 ml of sterile collection media to wash filter.

Centrifuge cells at 1400 rpm for 4 minutes. Decant the supernatant and resuspend the pellet in 10 mL of sterile collection media by pipetting up and down. If clumpy, filter again into new 50 ml conical tube. Centrifuge cells at 1400 rpm for 4 minutes. Decant the supernatant and resuspend the pellet in 2 mL of MesenPure media (MSC+ media)

Plate 1 ml of cell suspension into 2-10 cm plates and add 6 ml MesenPure media (MSC+ media) to each plate. Place in hypoxia (5% 02) incubator. After 3 hours of incubation, aspirate media and gently wash plate with PBS (repeat wash). Add 6 ml MSC+ media to plate and place in hypoxia (5% 02) incubator.

(4) For all Tissues:

After initial seeding do not disturb cells (i.e. wash or passage) for ˜2-3 days to allow the cells to fully attach. Monitor plates regularly to determine when to wash, add media, or passage.

For bone marrow, there are usually a lot of floating cells remaining in the plate. If so, change media on day 3 or 4. If there are minimal floating cells, perform a half media change (i.e. aspirate 3 ml media and add 3 ml fresh MesenPure media).

For lung and liver, plates become confluent more quickly and may need to be split as early as day 4. If more time is needed, perform a half media change on day 3 or 4.

(5) Passaging MSCs:

Wash cells once gently with 5-10 ml PBS (depending on plate size). Add 3-5 ml trypsin (depending on plate size) and incubate at 37° C. for ˜5 minutes. Bone marrow MSCs may take a bit longer to trypsinize. Monitor cell detachment under microscope every few minutes to reduce trypsin exposure time.

Add 5 ml MesenCult media and collect cells in conical tube. Confirm all cells have been collected off plate using microscope. Centrifuge at 1400 rpm for 4 minutes to pellet cells. Aspirate supernatant and resuspend cells in 1 ml MesenPure media. Depending on size of pellet (or cell count), plate cells onto a new tissue culture plate and place in hypoxia (5% O2) incubator. ˜1 million onto a T75; 3 million onto a T175

(6) Freezing MSCs:

Passage cells as described above and freeze ˜1-2 million cells in 90% FBS+10% DMSO as usual. Following thaw of MSC vial, cells are cultured in normoxia (for experimental simplicity, nitrogen costs, etc). MSCs do not change their expression phenotype when cultured at this stage in normoxia.

Isolated human mensenchymal stem cells (MSCs) are maintained and expanded in growth medium containing StemProMSC SFM XenoFree basal medium (StemCell Technologies) supplemented with StemProMSC SFM XenoFree supplement (StemCell Technologies) and 200 mM L-Glutamine. Isolated murine MSCs are maintained and expanded in growth medium containing MesenCult Basal Medium (StemCell Technologies) supplemented with MesenCult 1× supplement (StemCell Technologies) and MesenPure (StemCell Technologies).

Cells are transduced during the start of culture with a vector (e.g., lentivirus) comprising one or more transgenes.

Differentiation Protocol

Isolated murine or human (MSCs) are maintained and expanded in alpha MEM in BFGF 10 ng/ml or TGFβ 5 ng/ml, VEGF 5 ng/mL, and PDGFBB 10 μg/mL to differentiate MSCs into different stromal cell populations (e.g., conventional vascular smooth muscle cells, activated pericytes, myoepithelial cells, fibroblasts). Cells after treatment can then be switched to StemProMSC SFM media (StemCell Technologies) or alpha MEM for adoptive cell transfer.

Mice

6-10-week-old C57BL/6 and C57BL/6 Albino mice were purchased from Charles River at NCI Frederick. B6.SJL-Ptprca Pepcb/BoyJ (Pepboy), C57BL/6-Tg (TcraTcrb)1100Mjb/J (OT-I), and B6.Cg-Tg (TcraTcrb)425Cbn/J (OT-II) mice were purchased from the Jackson Laboratory. RAG^{-/-} OT-I mice were donated by Terry Fry. All experiments were approved by the NCI Animal Care and Use Committee and were conducted in specific pathogen-free conditions at the NIH Animal facility.

Cell Lines

M3-9-M embryonal rhabdomyosarcoma cells were derived as described in Meadors et al., Pediatr. Blood Center, 57, 921-929 (2011). KPC177669 pancreatic adenosarcoma cells were obtained from the NCI Center for Advanced Preclinical Research (CAPR). Lenti-X cells were provided by Terry Fry. All cell lines were verified via microarray analysis and routinely tested for mycoplasma. Tumor lines were stably-transduced with pFUGW-Pol2-ffLuc2-eGFP or pFUGW-Pol2-ffLuc2-mCherry and sterile-sorted by fluorescence activated cell sorting (FACS) to establish cell lines. M3-9-M-ffLuc2-mCherry cells were transduced with a retrovirus encoding ovalbumin (pMSCV-OVA) provided by Terry Fry, stained with PE anti-mouse H-2K^{b} bound to SIINFEKL (BioLegend), and sorted by FACS to establish the M3-9-M-ffLuc2-mCherry-OVA cell line. All tumor cell lines were cultured in complete RPMI. 10% FBS (Atlantic Biologicals), 1% glutamax (Gibco), 1% penicillin-streptomycin (Gibco), 1% non-essential amino acids (Gibco), 1% sodium pyruvate (Gibco), 1 mM HEPES (Gibco), and 50 μM 2-mercaptoethanol (Sigma) at 37° C. 5% CO₂.

Tumor Models

For M3-9-M orthotopic tumor experiments, mice were injected in the gastrocnemius muscle with 5×10⁵ M3-9-M cells in 100 μL of HBSS (Gibco). Primary tumors were measured two to three times a week and mice were monitored for survival over time. Tumor volume was calculated as 4/3π(X−X_{baseline})(Y−Y_{baseline}) (Z), where X, Y, and Z are the radius of each dimension of the mouse leg. For amputation experiments, primary tumors were surgically resected when tumor diameter was approximately 2 cm in the longest direction. For lung colonization experiments, 5×10⁴ M3-9-M-ffLuc2-mCherry cells were injected intravenously (i.v.) via tail vein in 200 μL HBSS. For KPC177669 tumor experiments, mice were injected intrasplenically. Briefly, mice were anesthetized with isoflurane and an 8-10 mm left subcostal incision was made. The spleen was exteriorized and 5×10⁵ KPC177669-ffLuc2-mCherry cells were injected in 100 μL HBSS, followed by an additional 100 μL of HBSS to flush the cells into the portal circulation. After two minutes, splenectomy was performed and the incision was closed in two layers.

Bioluminescent Tumor Cell Tracking

For metastasis experiments, lesions were detected by whole body in vivo and postmortem ex vivo organ bioluminescence imaging by an in vivo imaging system (IVIS). Anesthetized mice received a 100 μL intraperitoneal (i.p.) injection of 30 μg/mL D-luciferin (Gold Biotechnology) and were incubated for five minutes. For ex vivo tissue imaging, tissues were perfused with PBS, harvested, and incubated in 1 μg/ml D-luciferin in PBS for five minutes. Luminescence was detected by IVIS Lumina Series III (Perkin Elmer) for an exposure time of one minute. Display and image analysis were performed using Living Image Software (Perkin Elmer).

Lentivirus Production

Genetic constructs were synthesized by Genewiz and cloned into the lentiviral transfer vector pELNS. Lenti-X cells were cultured in high-glucose DMEM supplemented with 10% FBS, 25 mM HEPES, 2 mM L-glutamine, and 1% penicillin-streptomycin (Gibco) at 37° C. 5% CO₂. The day preceding lentivirus production, 1.8×10⁷ cells were seeded onto a 150 mm poly-D-Lysine coated plates (Corning). Cells were transiently transfected with Opti-MEM media (Gibco) containing Lipofectamine 3000 and P3000 (Thermo Fischer) 15 μg pRSV-Rev, 15 μg pMDLg/pRRE, 7.5 μg pMD2.G, and 22.5 μg pELNS for 6-8 hours, after which the transfection mixture was aspirated and replaced with fresh meda. Virus-containing supernatants were harvested at 24 hours and 48 hours, centrifugated to remove cellular debris, and stored at −80° C.

Tissue Processing

Lungs were perfused with PBS and inflated with digestion medium (HBSS supplemented with 1 mg/mL collagenase I, 20 μg/mL DNAse I and Dispase II). Single-cell suspensions were prepared by finely mincing tissues with a scalpel and incubating the tissue on a shaker at 37° C. for 20 minutes in 1 mL of digestion media. Tissue was passed through a 70 μM mesh strainer and washed with collection media twice. Tumors were disassociated using a modified protocol as described in Beury et al., J. Immunol, 196: 3470-3478 (2016). A fragment of the tumor was placed into a gentleMACs C tube (Miltenyi Biotech) containing 5 mL of digestion media. Tumors were then minced with scissors, processed on the gentleMACS dissociator (Miltenyi Biotech) using the program m_impTumor_02. Tubes were secured in an inverted position and agitated in a 37° C. shaker at 100 rpm for 40 minutes. Samples were processed again on the gentleMACS dissociator using program m_impTumor_03, passed through 70 μM cell strainer, and washed with collection media. For spleens and lymph nodes, tissues were mashed through a 70 μM cell strainer and washed with collection media. For spleen and tumor tissues, red blood cells were lysed with ACK lysis buffer (Life Technologies) for 5 minutes and washed with collection media.

Flow Cytometry

Cells were washed with PBS and stained with either Fixable Viability Dye e506 (eBiosciences) or Live/Dead Aqua (Thermo Fisher) for 30 minutes at 4° C. in the dark. Cells were then washed in FACS buffer (PBS supplemented with 1% BSA and 0.05% NaN₃), Fc blocking was performed with purified CD16/CD32 antibody (Invitrogen) and a combination of antibodies diluted in either FACS buffer or Brilliant Violet Stain Buffer (BD Biosciences) for 30 minutes at 4° C. in the dark. Cells were washed and, if necessary, fixed with 4% paraformaldehyde. Flow cytometry data were acquired on a BD LSR Fortessa or BD LSRII and analyzed with FlowJo software version 10.5 or greater (Tree Star). In all flow cytometry assays, manual gating was based on fluorescence minus one (FMO) controls.

Chemotherapy Treatment

Cyclophosphamide monohydrate (Sigma Aldrich) was prepared to a final concentration of 20 mg/mL in PBS and passed through a 22 μm filter. Fludarabine phosphate (Actavis Pharma, Inc.) was reconstituted with sterile PBS to a final concentration of 50 mg/mL. 100 μL was injected per mouse i.p. 48 hours prior to IL12-GEMy transfer.

Antibody Depletion

Mice were injected with 100 μL depletion antibodies i.p. An initial depletion with 200 μg of anti-CD8a antibody clone 2.43, anti-CD4 antibody clone GK1.5, or rat IgG2b isotype control clone LTF-2, or 100 μg of anti-NK1.1 (PK136) (BioXCell) was administered on days 9, 11, and 12 post tumor inoculation. Antibody depletion treatment continued with the administration of 200 μg of antibody every 3-5 days for the duration of the experiment.

T Cell Activation

Spleens from OT-I or Rag^{-/-} OT-I mice were harvested and processed into single cell suspension as described above. Splenocytes were activated in complete RPMI in the presence of 50 U/mL IL-2 and 1 μg/mL OT-I cognate peptide OVA₂₅₇₋₂₆₄ (SIINFEKL) or OT-II cognate peptide OVA₃₂₃₋₃₃₉ for four days. Activated OT-I T cells were transferred into mice i.v. via tail vein.

Immunofluorescence

Lungs were harvested, embedded fresh-frozen in OCT, and sectioned. CODEX analysis was performed in collaboration with the NCI Collaborative Protein Technology Resource according to published protocols (Akoya Biosciences) (Goltsev et al., Cell, 174: 968-981 e915 (2018)). Images were prepared using the Palantir Foundry image viewing and analysis platform developed for the NIH (Palantir Technologies).

Bulk RNA Sequencing

Naïve or tumor-bearing mice were treated or not with 8×10⁶ IL12-GEMys on day twelve post tumor inoculation. Mice were euthanized three days after IL12-GEMy transfer and lungs were harvested. For bulk RNA sequencing, lungs were flash-frozen in liquid nitrogen. Tissue was homogenized in TRIzol (Thermo Fisher) and RNA was isolated by chloroform extraction followed by the RNeasy Mini Kit (Qiagen) according to manufacturer's recommendations. 3′ library preparation was performed using the Illurnina TruSeq Stranded rnRNA kit and Hiseq4000 platform (Illumina) at a total of 100 million reads per sample according to standard operating procedure at the CCR Genomics core facility. Alignment, normalization, and primary gene expression analysis were performed as described in Murgai et al., Nat. Med., 23: 1176-1190 (2017) utilizing the computational resources of the NIH HPC Biowulf cluster (http://hpc.nih.gov). Pathway analysis was performed using Ingenuity Pathway Analysis (Qiagen) on gene sets with a greater-than-two-fold change and p-value cut off of 0.05.

Single Cell RNA Sequencing

Lungs from naïve or tumor-bearing mice with or without IL12-GEMy treatment (n=4 mice per group) were processed into single-cell suspension and oligodT-based cDNA libraries were barcoded by droplet-partioning using the Chromium Single Cell Controller (10× Genomics) system at the NCI-CCR Single Cell Analysis Facility. Dead cell removal was performed and samples were incubated with TotalSeq-A hashtag oligos (HTOs) (BioLegend). Two biological replicates were run together per capture lane. Sequencing was performed on the NovaSeq (Illumina) at the NCI-CCR Sequencing Facility. Sequencing read demultiplexing, alignment to mm10 (Ensembl Ref annotation 93), and generation of a unique-molecular-index-collapsed gene expression matrix was performed using cellranger version 3.0.2 (10× Genomics). Additional data processing and analysis was performed using Seurat v3.0.2 in RStudio running R v3.6.0. In short, each sample set was filtered for cell barcodes with greater than 500 genes detected and less than 20% percent mitochondrial gene expression. Cell barcodes with either no cell hashing antibody detected or multiple were excluded from the analysis. Data from all samples were merged, and normalization and scaling was performed using SCTransform. Clustering and UMAP projections were performed on combined data, with collapsing of biologically-relevant clusters informed by clustree empirical analysis. Cell type/cluster marker gene detection was performed with FindAllMarkers in Seurat with Wilcoxon ranked sum test. Differential expression testing across conditions for each cluster performed with MAST. Single cell plots generated within Seurat using ggplot.

Statistical Analysis

All statistical analysis was performed in Prism version 7.03 or greater (GraphPad Software). Graphs represent mean values±standard error. P values were calculated for bar graphs using unpaired two-tailed Student's t test, one-way ANOVA, or log-rank statistics for survival analyses in Prism as indicated in FIG. legend. Error bars represent standard error. In boxplots, the center line represents the median, the box limits denote the 25^{th} to the 75^{th} percentile and the whiskers represent the minimum and maximum value. p<0.05 was considered statistically significant. * p≤0.05, ** p≤0.01, *** p≤0.001, **** p≤0.0001.

### Example 1

This example demonstrates that a core program of immune suppression is consistent with a stem cell-like niche.

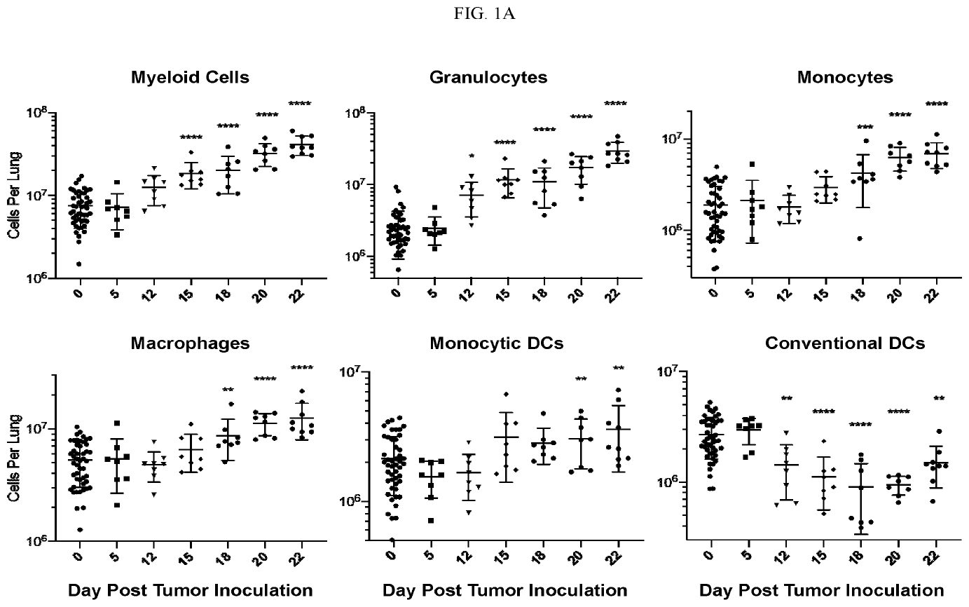

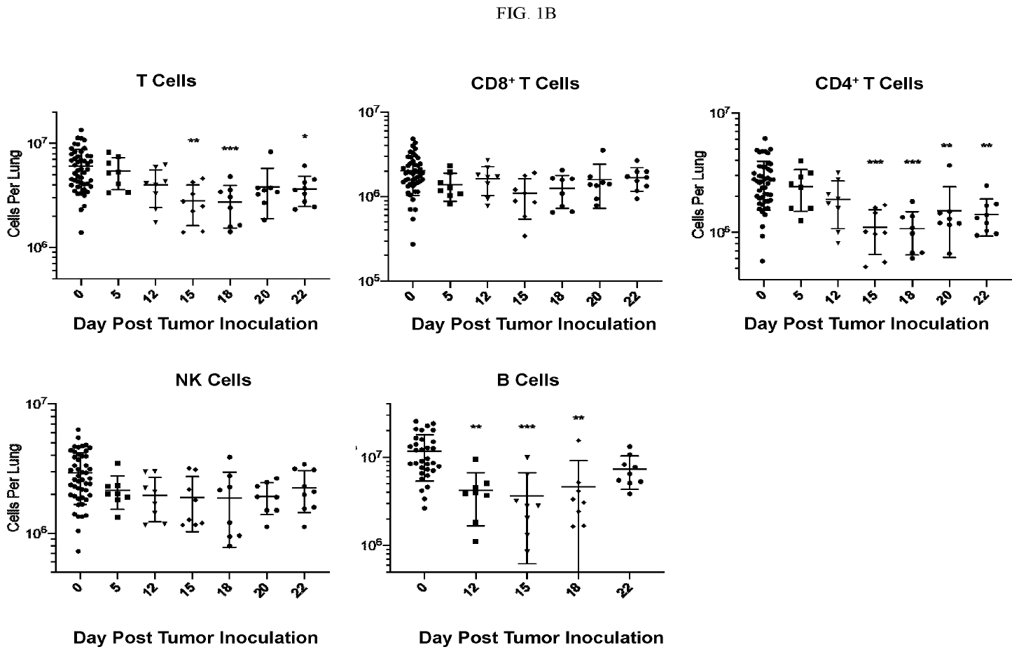

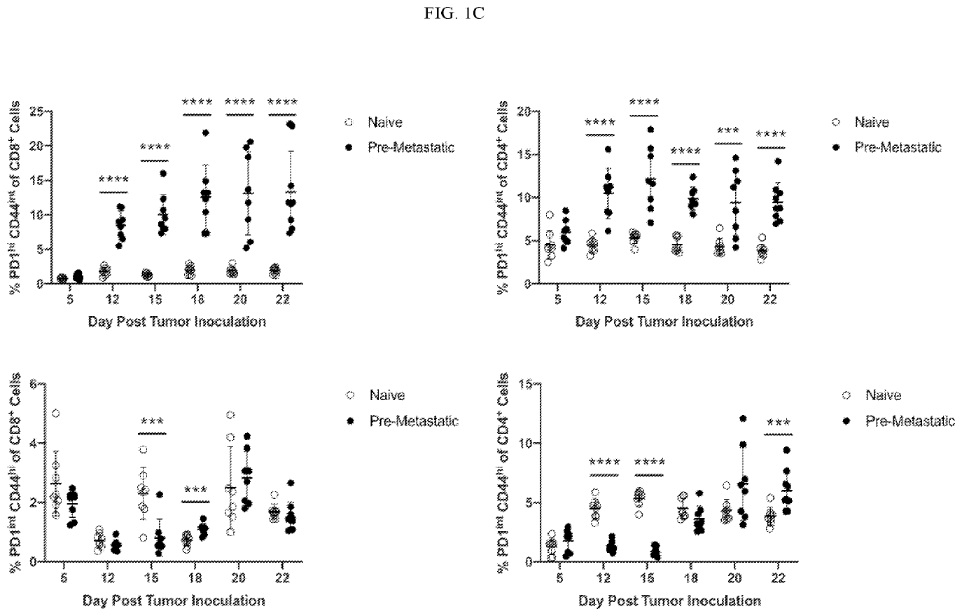

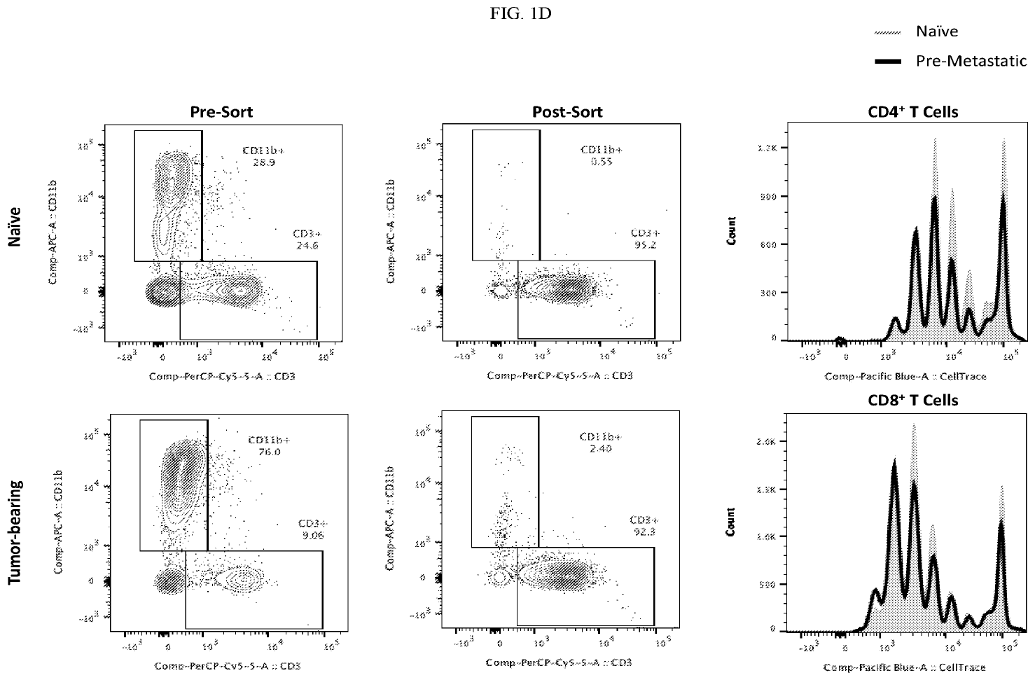

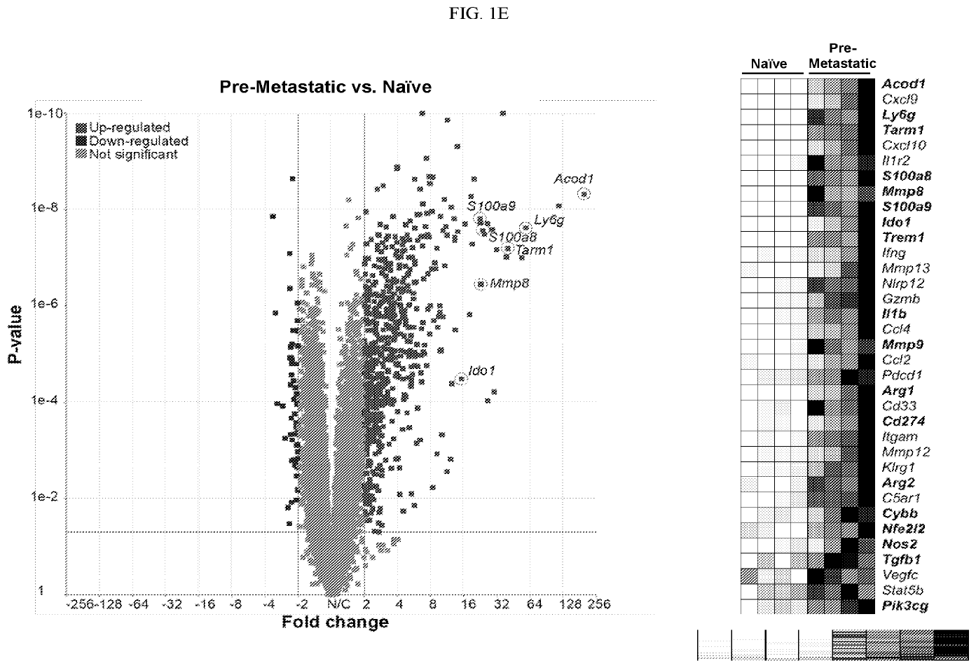

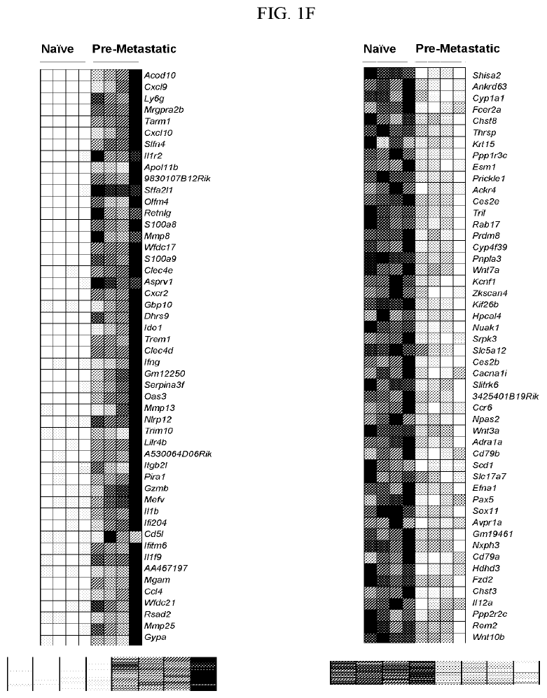

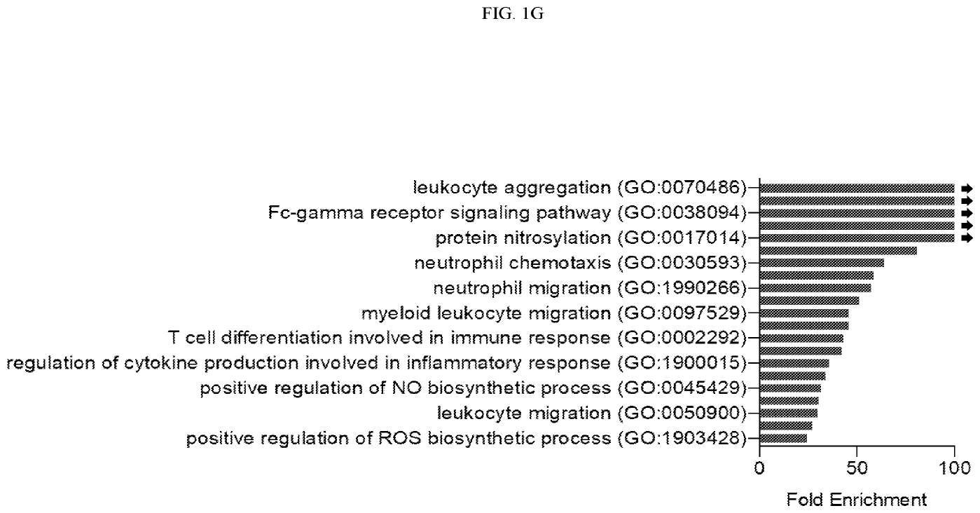

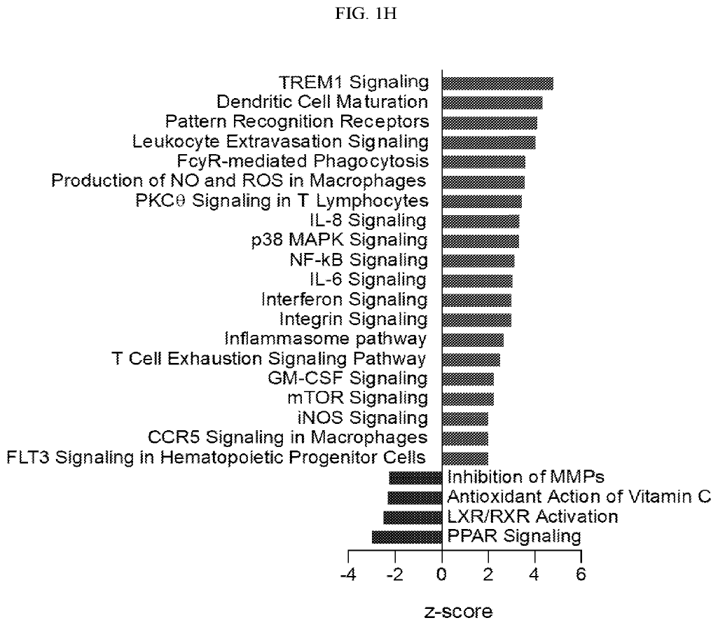

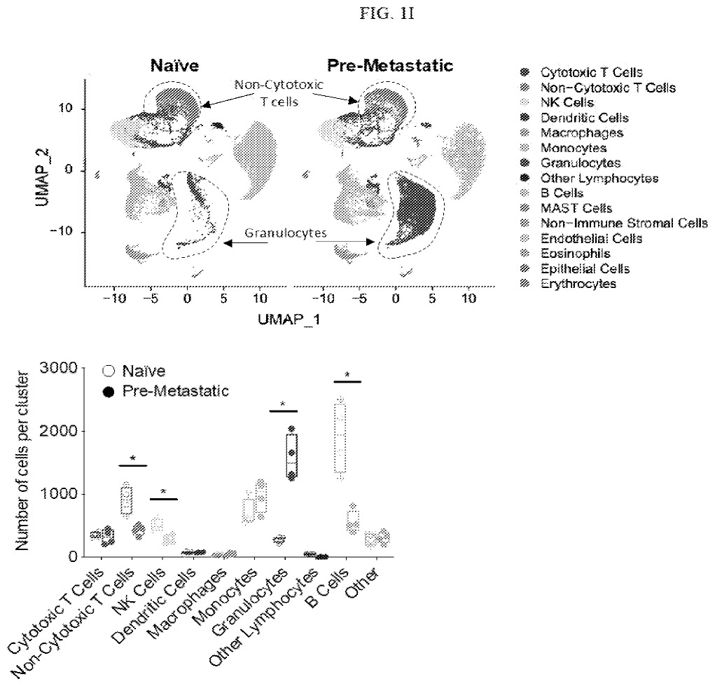

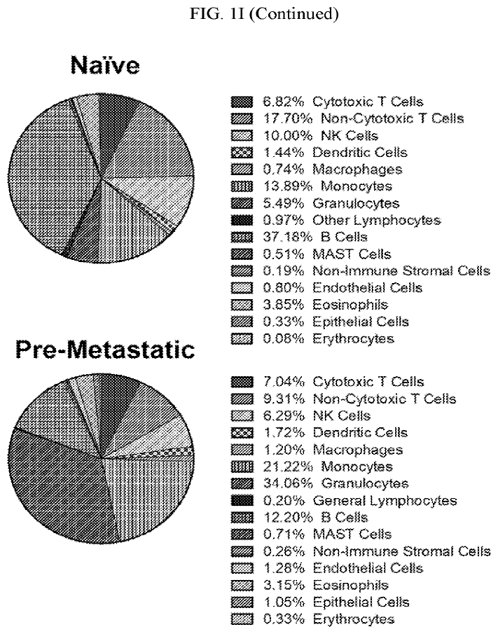

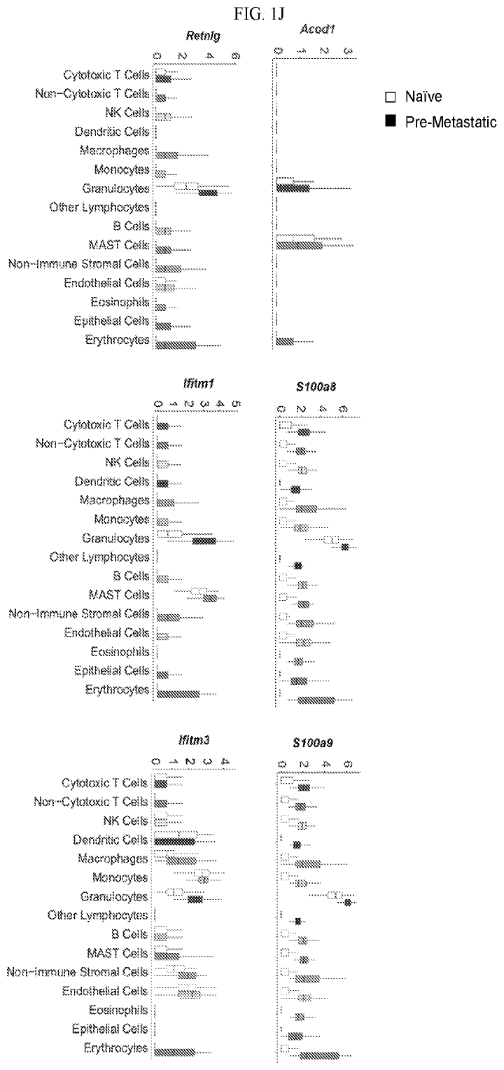

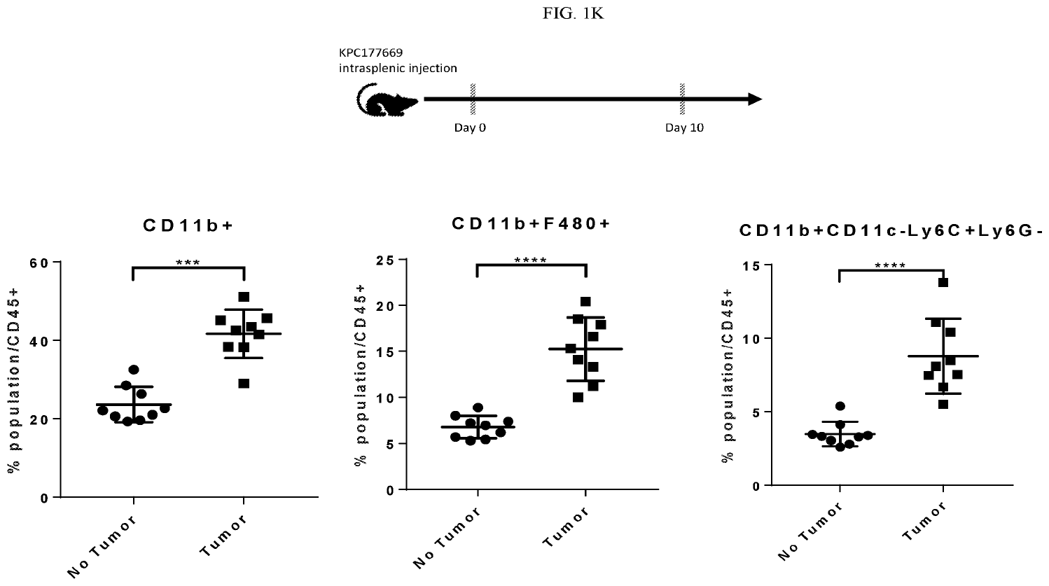

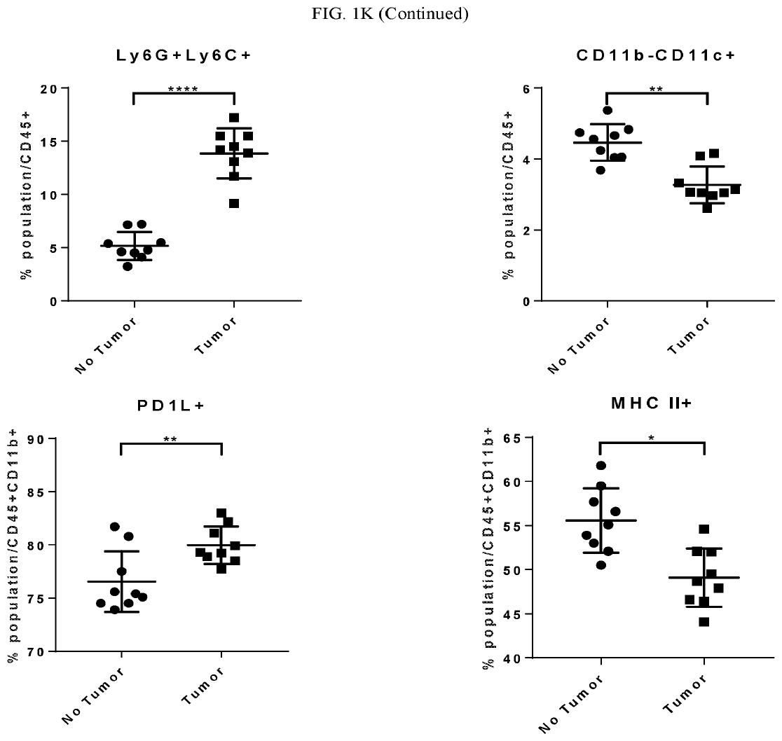

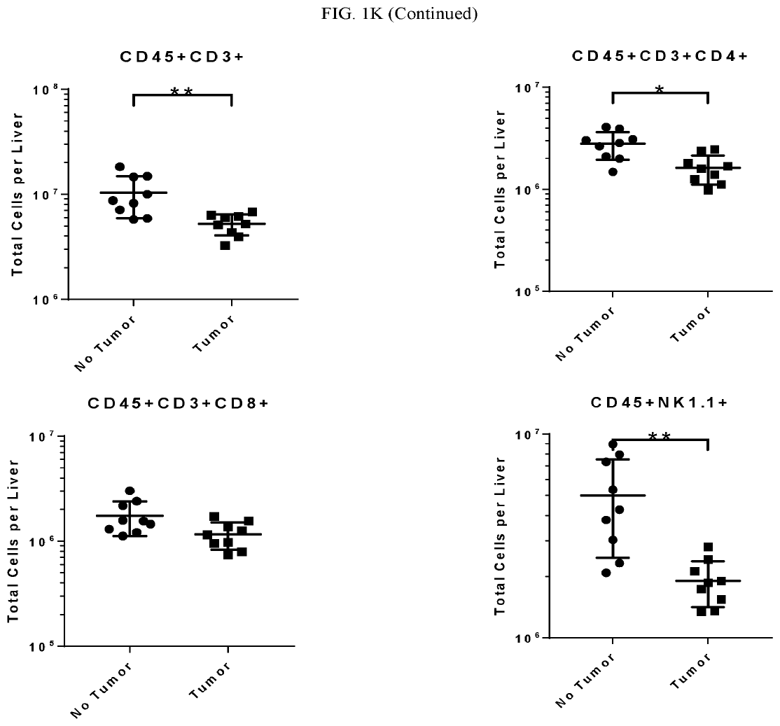

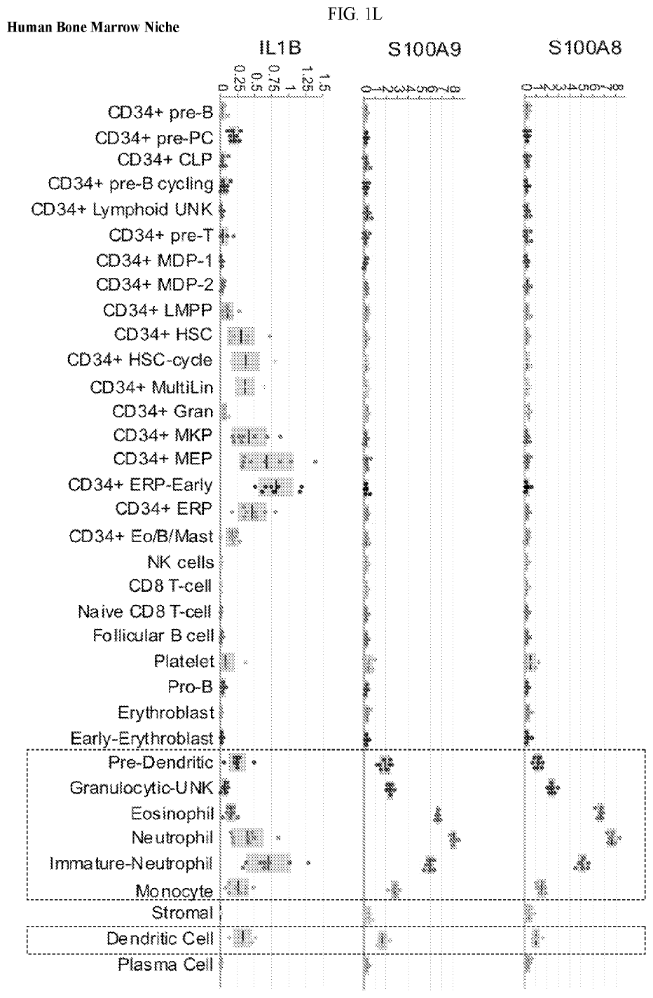

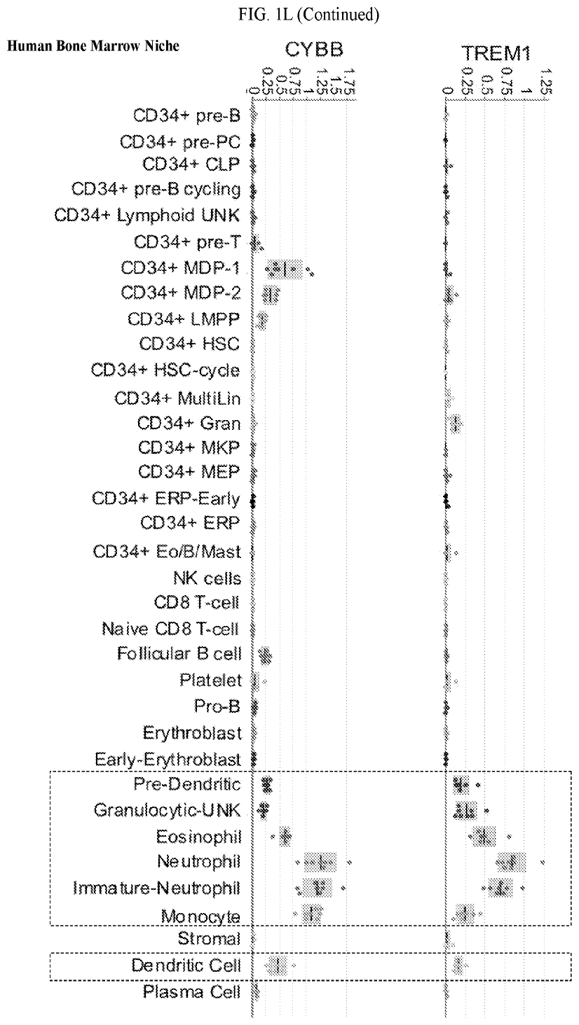

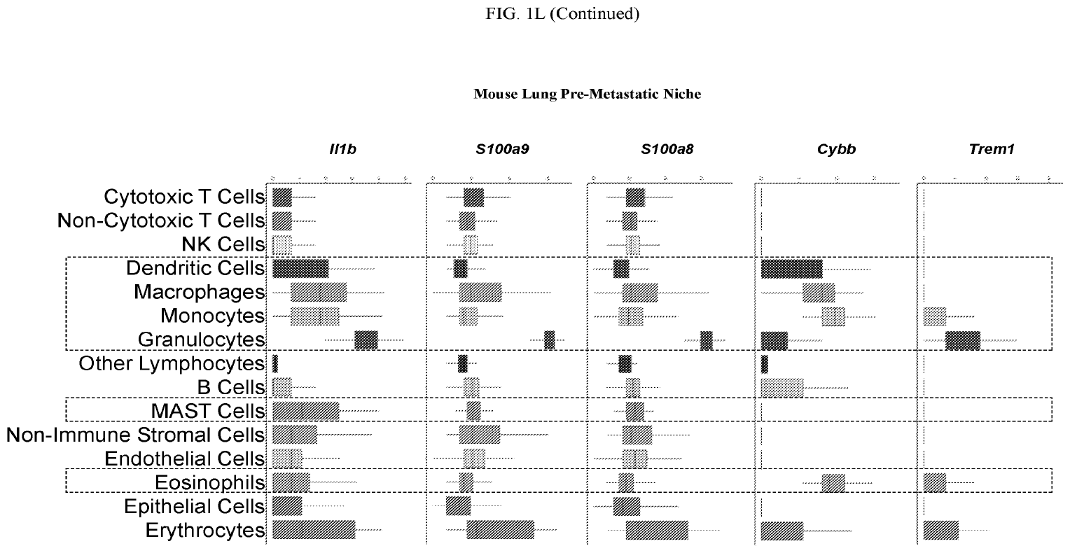

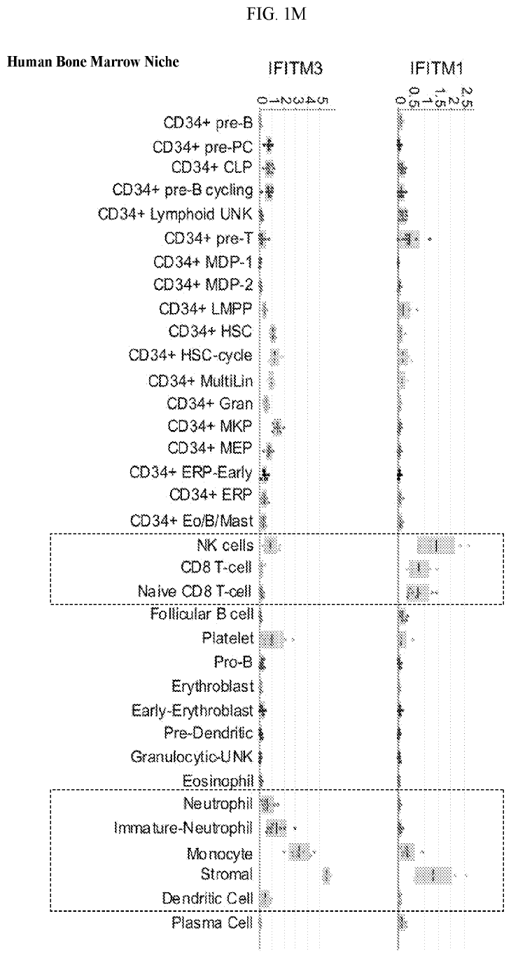

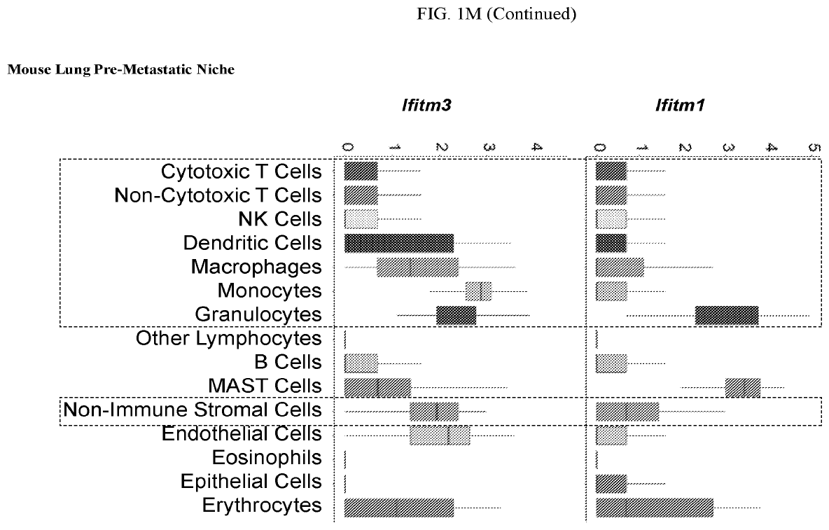

To explore how diverse immune populations are transformed at distant metastatic sites in response to primary tumor growth during metastatic progression, M3-9-M, an orthotopic syngeneic tumor model of rhabdomyosarcoma that reliably metastasizes to the lungs and is highly analogous to human metastatic rhabdomyosarcoma, was utilized. Immune population dynamics in the pre- and early metastatic lungs of tumor-bearing mice were analyzed by flow cytometry (FIGS. 1A-C).

Naïve mice were taken at each time point and are indicated as day zero post tumor inoculation. A significant increase in the number of myeloid (CD11b⁺) cell populations, including granulocytes (CD11b⁺Ly6G⁺), monocytes (CD11b⁺Ly6G⁻Ly6C⁺), macrophages (CD11b⁺F4/80⁺), and monocytic dendritic cells (CD11b⁺CD11c⁺), was observed in the lungs of tumor-bearing mice (FIG. 1A). Interestingly, a dramatic decrease of conventional DCs (CD11b⁻CD11c⁺) was observed, suggesting that antigen presentation and priming of an effective adaptive immune response is diminished.

In parallel with the observed increase in myeloid populations, there was a marked reduction in the number of lymphocytes including total T cells (CD3⁺), CD4⁺ T cells (CD4⁺CD3⁺), as well as B cells (CD19⁺) with increasing primary tumor burden, while the number of NK cells (CD3⁻NK1.1⁺) remains unchanged (FIG. 1B). The PD1^{hi}CD44^{int} population, associated with more dysfunctional T cells, is most significantly increased in the premetastatic lung. Interestingly, the percentage of activated PD1^{int}CD44^{hi} cells is lower in premetastatic lungs than in naïve lungs at early time points (day 15) (FIG. 1C). Although T cell numbers were diminished, those T cells that were found within pre-metastatic lungs retain their proliferative capacity when isolated from the pre-metastatic microenvironment and activated in vitro, suggesting that local signals within the pre-metastatic milieu suppress T cell activity rather than an intrinsic defect in T cell function (FIG. 1D). These data demonstrate the development of a myeloid-rich, T cell-poor environment that is formed at metastatic sites during disease progression from pre-metastatic to late metastatic stages.

In order to investigate the transcriptional programs that underlie the metastatic niche environment, deep transcriptional analysis of whole lungs from naïve and pre-metastatic mice fifteen days post primary tumor inoculation was performed. A dramatic shift in the expression of many genes in the lungs was observed in response to the presence of a distant tumor, with many more genes being upregulated than downregulated (FIG. 1E). The top fifty genes upregulated and downregulated in pre-metastatic compared to naïve lungs are shown (FIG. 1F). Genes associated with immune activation (Cxcl9, Tarm1, Ifng, Gzmb, Pdcd1, and Klrg1) were significantly increased in tumor-bearing mice compared to naïve mice, demonstrating an immune response to the tumor. Most notably, however, a strong gene signature associated with immune suppression (Acod1, Ly6g, S100a8, S100a9, Mmp8, Mmp9, Ido1, Trem1, Il1b, Arg1, Arg2, Cd274, Cybb, Nfe2l2, Nos2, Tgfb and Pik3cg) was identified and this core transcriptional program is one of the most highly upregulated functional features of pre-metastatic lungs.

To investigate the functional implications of the most altered gene programs, gene ontology (GO) analysis of the top fifty genes upregulated in pre-metastatic lungs was performed, which revealed the enrichment of many immunosuppressive biological processes (FIG. 1G). Among these, leukocyte and neutrophil migration and aggregation indicate enhanced expression of genes that promote the intravasation of neutrophils into the metastatic niche, while nitric oxide (NO) and reactive oxygen species (ROS) biosynthesis and protein nitrosylation inhibit T cell receptor signaling and T cell activation. To learn more about the underlying biology of the most differentially-expressed genes between pre-metastatic and naïve lungs, pathway analysis of the gene set significantly upregulated in pre-metastatic lungs was performed (FIG. 1H). Pathway analysis indicated a significant enrichment in multiple pathways of myeloid-cell mediated immune suppression, including production of NO and ROS, p38 MAPK signaling, granulocyte-macrophage colony-stimulating factor (GM-CSF) signaling, the inflammasome pathway and inducible nitric oxide synthase (iNOS) signaling (FIG. 1H). T cell exhaustion-associated pathways were also enriched in the pre-metastatic lungs, and T cell activation genes were downregulated in the pre-metastatic setting such as IL12a, Tril and Ccr6, implicating suppression of adaptive immune responses during metastatic development. Furthermore, anti-inflammatory, anti-angiogenic pathways liver X receptor (LXR)/retinoid X receptor (RXR) and peroxisome proliferator-activated receptor (PPAR) signaling were significantly downregulated in the lungs of tumor-bearing mice, which are pathways involved in lipid metabolism, suppression of tumor growth, reduction of MDSC abundance and dampening inflammation in the bone marrow niche. LXR agonists, such as RGX-104, have begun to be used in clinical trials in patients with metastatic solid tumors to reduce MDSC expansion (ClinicalTrials.gov ID: NCT02922764).

Together, the transcriptomics data support a core functional module of immune suppression which we define as the core immune suppression program within the pre-metastatic niche represented by the upregulation of Acod1, Ly6g, Tarm1, S100a8, S100a9, Mmp8, Mmp9, Ido1, Trem1, Il1b, Arg1, Arg2, Cd274, Cybb, Nfe2l2, Nos2, Tg1b1 and Pik3cg (FIG. 1E). This pre-metastatic niche gene signature is a marker of metastasis and can serve as a readout of response to microenvironment targeted therapy.

To further elucidate the cellular source of our transcriptional immune suppression niche program in the lungs of tumor-bearing hosts, scRNA-seq of naïve and pre-metastatic lungs at day fifteen post tumor engraftment was performed. Cell type identity of each cluster was defined by the expression of lineage markers enriched in each cluster. ScRNA-seq revealed a global shift in the relative abundance of specific immune cell populations. Consistent with the flow cytometric data, a striking expansion of the number of granulocytes was observed along with a significant decrease in lymphocytes including non-cytotoxic T cells, NK cells, and B cells compared to naïve lung (FIG. 1I). Myeloid cell populations demonstrate an upregulation of many of the genes responsible for the immune suppression signature seen in the whole-lung pre-metastatic niche including Acod1, S100a8 and S100a9 (FIG. 1J). Additionally, one of the most highly upregulated genes in pre-metastatic lungs, Retnlg, is expressed in almost all cell types examined and has been reported to be involved in promyelocytic differentiation and myeloid cell chemotaxis (FIG. 1J).

Many of the upregulated genes in the pre-metastatic lungs of tumor-bearing mice compared to naïve mice have previously been implicated in metastatic niche biology (S100a8, S100a9, and Mmp9). However, a new immunoregulatory set of genes was discovered to be upregulated in many cell clusters in the pre-metastatic niche: IFN-inducible transmembrane (IFITM) genes Ifitm1 and Ifitm3 (FIG. 1J). These interferon-stimulated genes (ISGs) are activated in response to nucleic acids in extracellular vesicles and from apoptotic cells in the context of tissue damage, as often occurs in cancer. These genes are highly evolutionarily conserved to protect stem cells from viral invasion by preventing endocytic fusion events of viral entry and have developed within the stem cell niche to negatively-regulate type I interferon responses that could potentially damage the stem cell pool. These same genes are found to be upregulated in the pre-metastatic niche, where it is believed that they attenuate interferon-mediated immune responses to cancer. Drawing further parallels between stem cell niches and the pre-metastatic niche, the absence of T cells in neurogenic niches is considered a sign of a healthy niche, while the infiltration of T cells in old neurogenic niches is associated with loss of niche function, recapitulating the discovery of T cell exclusion that was observed during pre-metastatic niche formation.

The liver is another common site of metastasis in many cancers. Using an orthotopic pancreatic tumor model that metastasizes to the lungs, the immune populations in the liver at a pre-metastatic time point (day 10) were analyzed (FIG. 1K). The same trends that we observed in pre-metastatic lungs are present in pre-metastatic liver, mainly, an increase in myeloid cells, a decrease in dendritic cells and antigen presentation, as well as a decrease in T cells, mostly CD4+ T cells.

Based on this data, the inventors propose that myeloid cells play an important role in creating an immune privileged niche environment to protect stem cells from immune attack. Therefore, to test the contribution of this transcriptional regulatory niche program present in the in the human setting, the core immune suppression program in the human hematopoietic stem cell niche was explored (FIG. 1L). Indeed, a high degree of similarity was found in many of the myeloid-associated immune suppression genes found in the bulk sequencing data in human bone marrow myeloid populations from the hematopoietic stem cell niche (TRFMI, CYBB, S100A8, S100A9 andILIB) as well as in single cell data. Although this transcriptional signature is most notably upregulated in myeloid cell populations, it is important to acknowledge that multiple other cell types also contribute to this immunosuppressive program of the pre-metastatic niche, consistent with the role of other cell types in creation of a stem cell niche environment. Furthermore, IFITM1 and IFITM3 are also upregulated in the human bone marrow niche with a similar expression pattern in myeloid, stromal and lymphocyte populations as in the pre-metastatic niche, validating this niche gene program which includes highly-conserved mechanisms of stem cell protection (FIG. 1M). This identified core immune suppression program with a shared transcriptional signature in niche biology is created in the lung during the early stages of metastatic development, implicating immune suppression and enhanced niche formation as essential in the process of metastatic progression. The immune suppression signature (FIG. 1E-F) in lung and other pre-metastatic organs such as liver in pancreatic cancer setting marks dysregulated niches and the metastatic process.

### Example 2

This example demonstrates the generation of genetically-engineered myeloid cells (GEMys) to modulate the key regulatory programs in the pre-metastatic environment.

Myeloid cells are elevated in circulation and highly enriched in the tumor and metastatic microenvironments in both mice and humans (FIG. 1A). To take advantage of this marked infiltration of myeloid cells into pre-metastatic lungs, GEMys were generated as a novel platform to deliver cargo to manipulate the cellular crosstalk in the niche microenvironment. As IL-12 has been shown to demonstrate potent antitumor activity, GEMys were designed to produce IL-12 (IL12-GEMys) as proof-of-principle to evaluate the functional impact of reversing the core immune suppression gene niche program in the metastatic microenvironment.

IL12-GEMys were generated by transduction of lineage-depleted bone marrow cells with lentivirus encoding the potent antitumor cytokine IL-12 (FIG. 2A). These cells were cultured in media promoting myeloid cell expansion for four days, the time point that yielded the greatest expansion of cells. The IL-12-producing construct co-expresses Thy1.1 to enable IL12-GEMy tracking and evaluate transduction efficiency. The IL12-GEMy product is a heterogeneous population of myeloid cells with a large component of Ly6G⁻ Ly6C⁺ monocytes with transduction efficiency ranging from 25%-50% (FIG. 2B). IL-12 production by IL12-GEMys in culture was confirmed and found to be directly related to viral titer by ELISA (FIG. 2C).

IL12-GEMys were found primarily in the liver, lung, and spleen in similar proportions as vector control cells, indicating that cell-autonomous homing behaviors were not disrupted by IL-12 expression and illustrating the ability of these cells to home to many sites (FIG. 2D). Both IL12-GEMys and vector control myeloid cells are found at very low frequencies in the bone marrow, tumor, lymph nodes and circulating blood. Pie charts showing the change in phenotype of the myeloid populations present in the lung as a percentage of CD11b⁺ cells demonstrate that IL12-GEMys retain a Ly6C⁺Ly6G⁻ monocytic phenotype in the lungs, suggesting that their phenotype is not drastically changing in vivo (FIG. 2D). Upon intravenous (i.v.) transfer of IL12-GEMys into tumor-bearing mice, IL-12 could not be detected in the plasma above baseline levels however was detected in the lung tissue, suggesting that the secreted IL-12 is limited to the microenvironment and does not accumulate to significant systemic levels (FIG. 2E). This is an advantage of the IL12-GEMys compared to systemic IL-12 administration, which has shown dose-limiting toxicity in the clinic (Leonard et al., Blood, 90: 2541-2548 (1997)). Together these data demonstrate that IL12-GEMys home to pre-metastatic lungs and can be exploited to deliver cytokines to reprogram the core immune suppression program in the pre-metastatic microenvironment. These studies are the first to demonstrate the feasibility of generating functional murine GEMys from hematopoietic stem and progenitor cells in the metastatic microenvironment.

### Example 3

This example demonstrates that IL12-GEMys restore and activate T cell populations in pre-metastatic lungs.

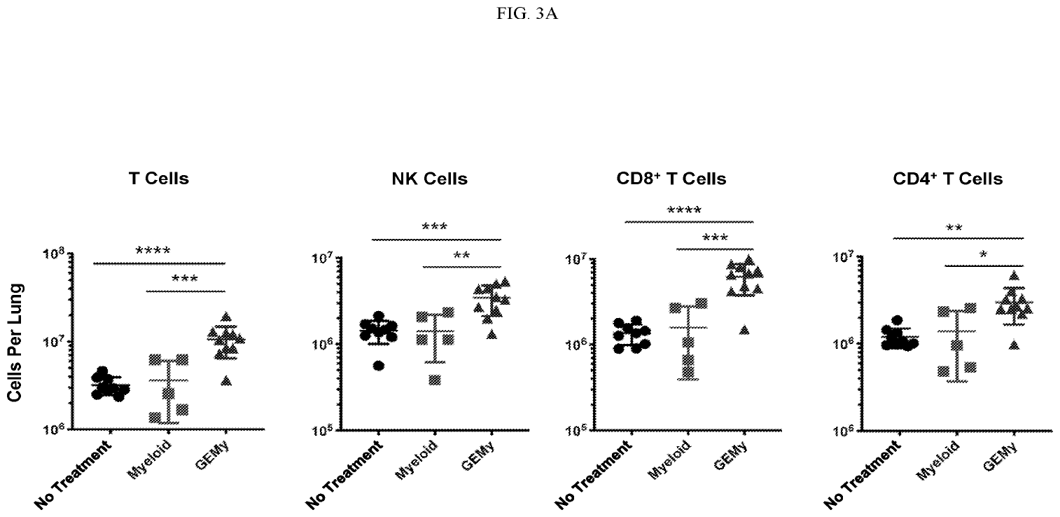

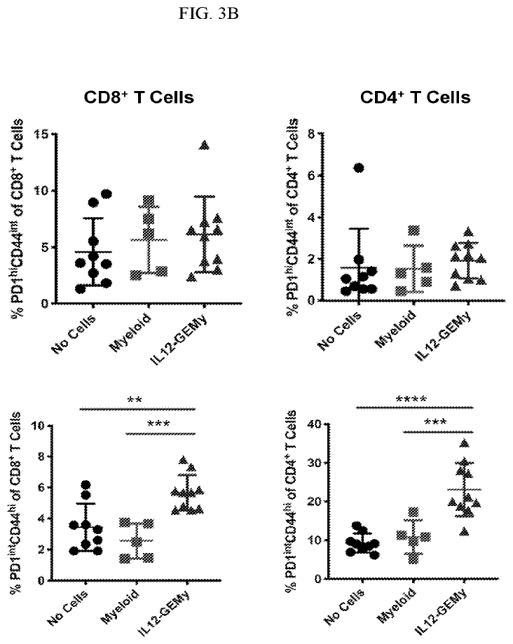

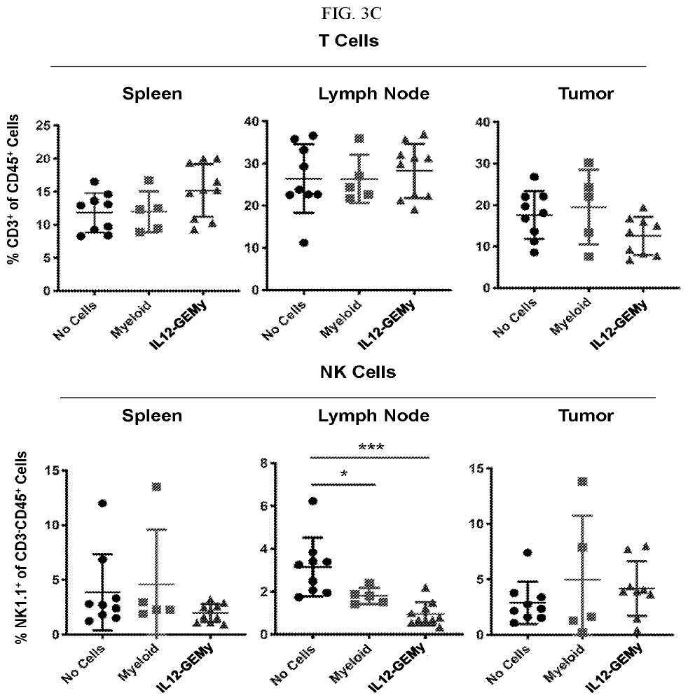

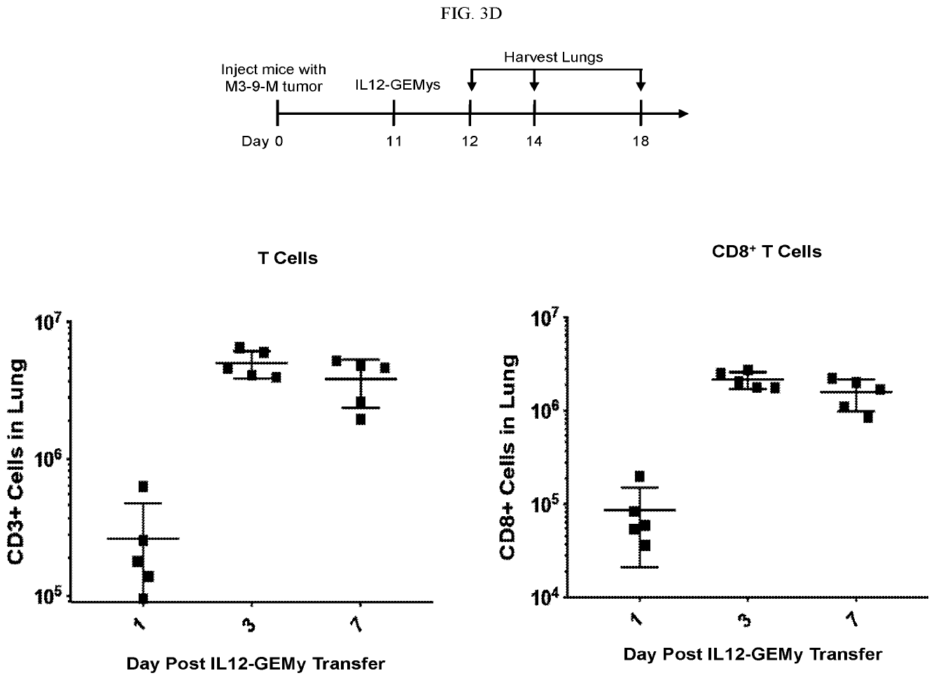

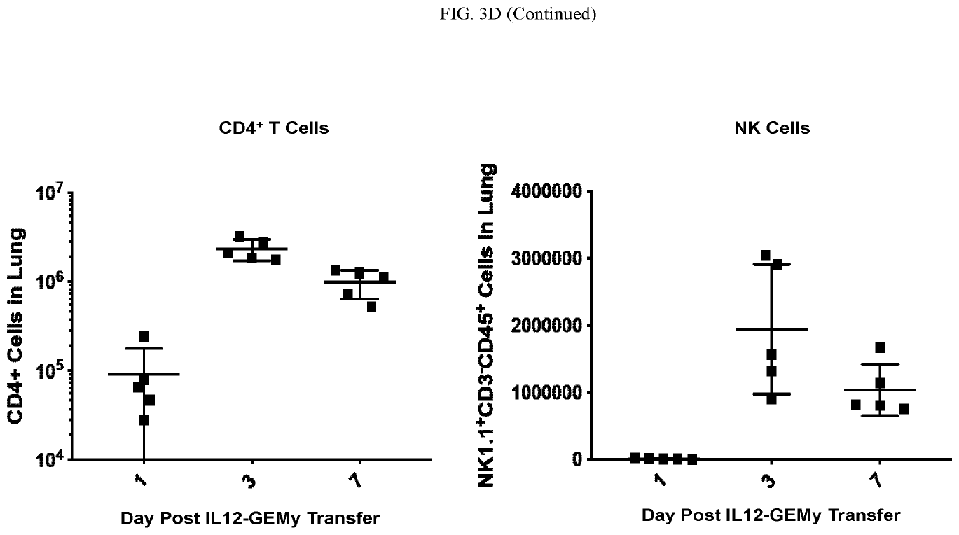

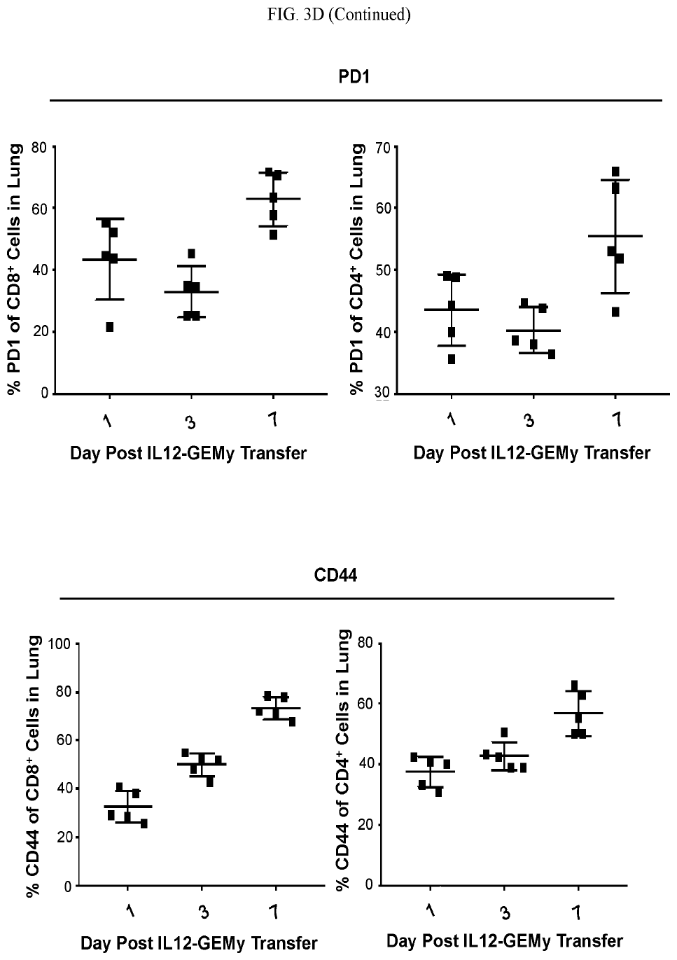

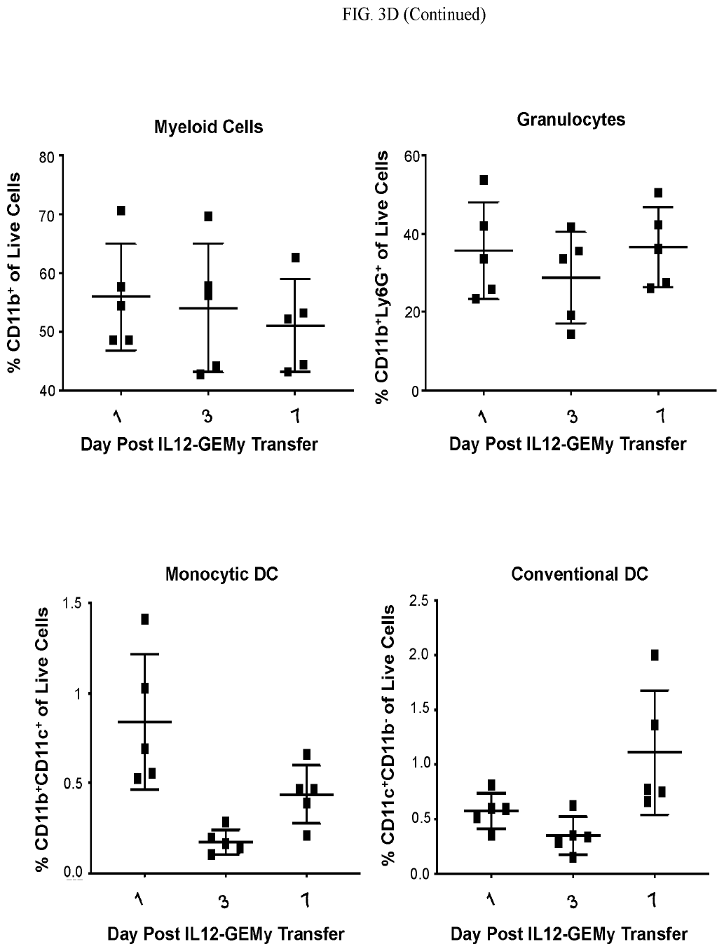

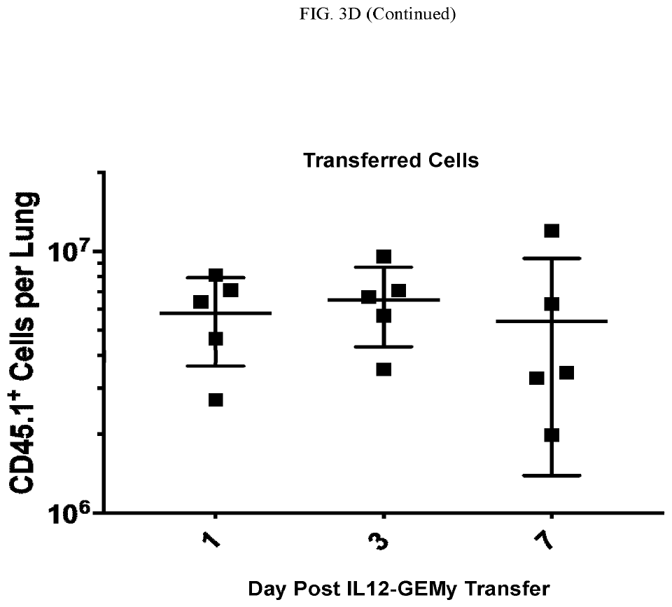

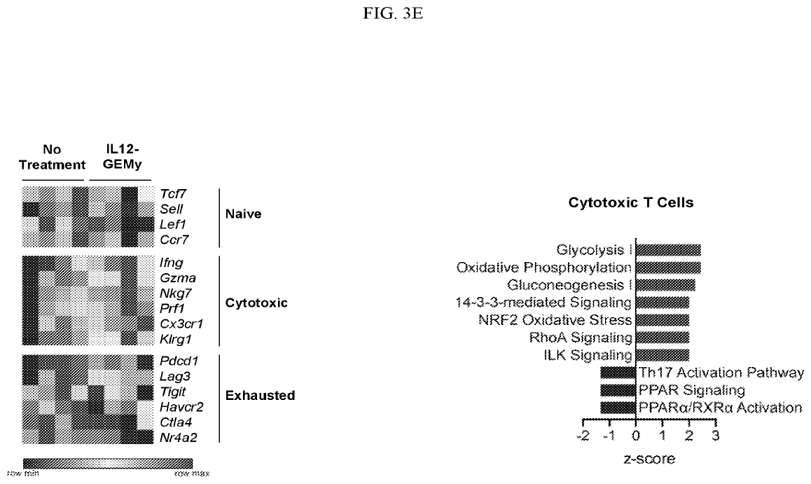

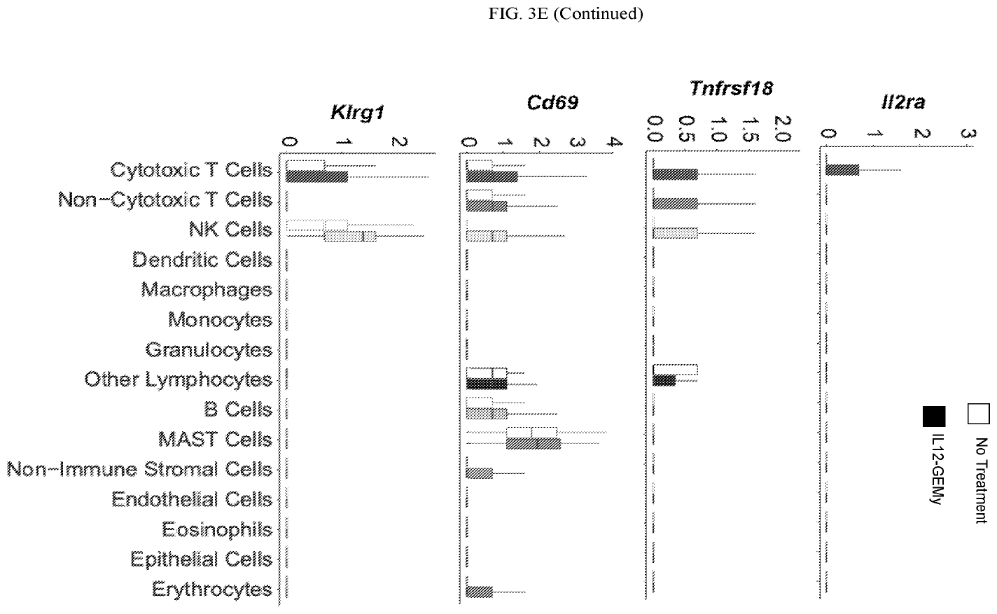

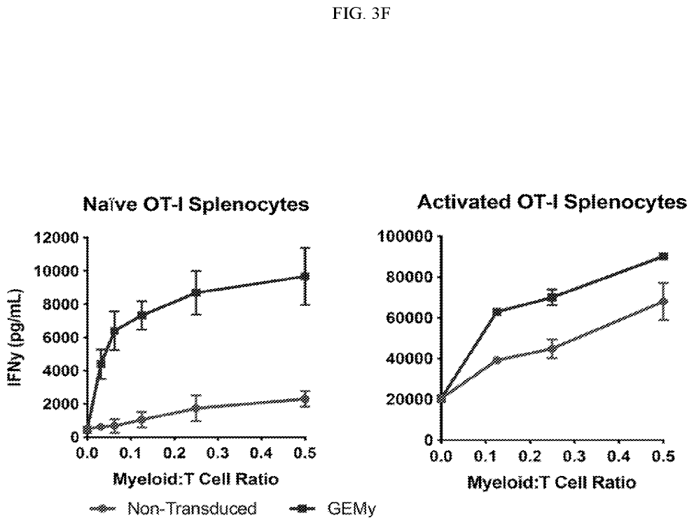

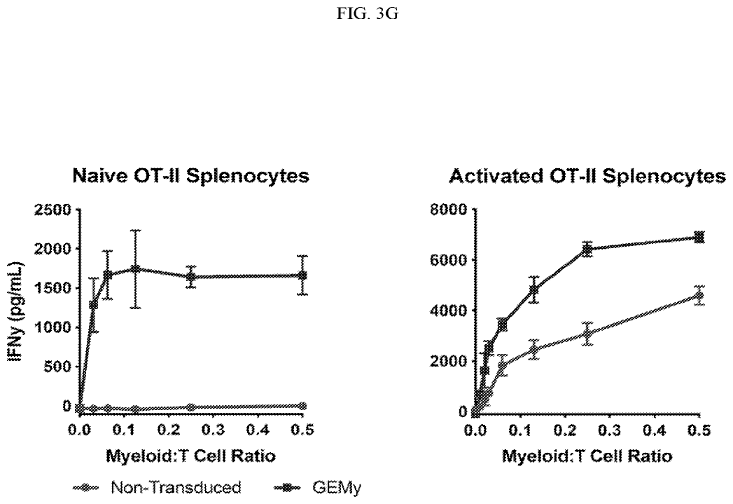

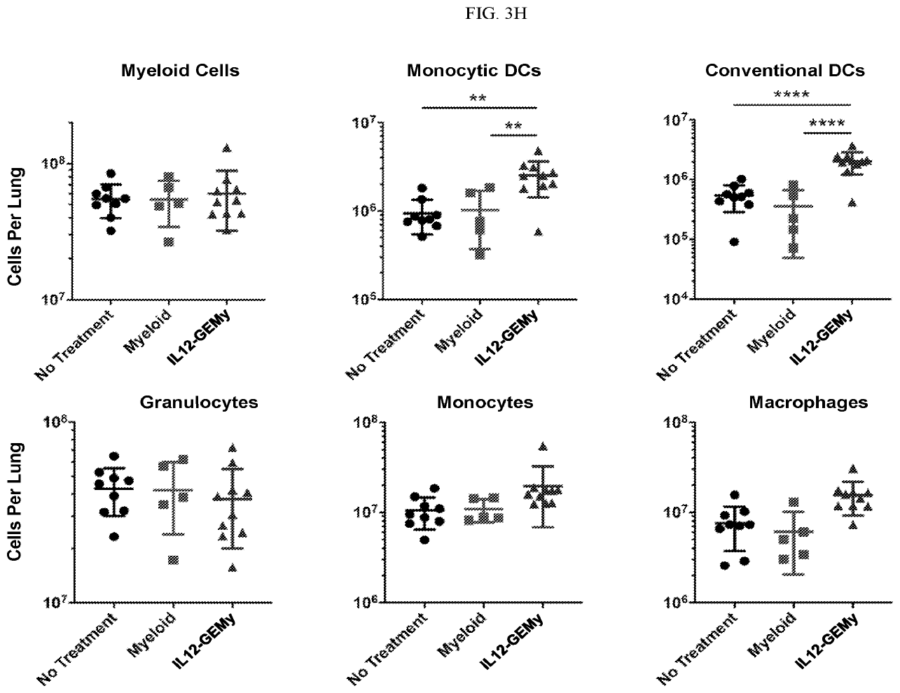

To determine the impact of IL12-GEMys on the immunosuppressive metastatic microenvironment, lymphoid and myeloid populations in the lungs of mice at primary tumor endpoint (day 27) that received either no treatment, non-transduced myeloid cells, or IL12-GEMys were profiled. The lungs of tumor-bearing mice that received IL12-GEMys had significantly more CD8⁺ T cells, CD4⁺ T cells and NK cells compared to either mice receiving no treatment or non-transduced myeloid cells (FIG. 3A). Additionally, the percentage of CD4⁺ and CD8⁺ T cells that express PD1^{int}CD44^{hi} was also significantly increased in the lungs of IL12-GEMy-treated mice, indicating that T cells from IL12-GEMy-treated mice display a more activated phenotype (FIG. 3B). There were no appreciable differences in the frequency of CD3⁺ and NK1.1⁺ cells in the spleen, tumor-draining lymph node, or tumor, with the exception of decreased NK1.1⁺ cells in the tumor-draining lymph node of IL12-GEMy-treated mice, indicating a metastatic niche-specific response to IL12-GEMy treatment in tumor-bearing mice (FIG. 3C).

To further delineate the kinetics of T cell recruitment or expansion, lungs were harvested from M3-9-M tumor-bearing mice that were treated with IL12-GEMys and analyzed by flow cytometry at pre- and early metastatic time points (FIG. 3D). The number of T cells and NK cells increased as early as three days post-IL12-GEMy transfer (FIG. 3D), while the greatest increase in the proportion of PD-1+ and CD44+ T cells occurred between three to seven days after IL12-GEMy delivery (FIG. 3D).

Together, these data demonstrate that T cell recruitment and/or expansion occurs early in response to IL12-GEMy treatment and results in subsequent T cell activation that persists to limit metastatic progression.

Whole lung deep transcriptomic profiling of the lungs of IL12-GEMy-treated mice demonstrates low expression of genes associated with naïve T cells and elevated expression of genes associated with cytotoxicity, suggesting that IL12-GEMy cell therapy promotes the activation of cytotoxic T cell responses (FIG. 3E). Furthermore, the expression of genes associated with T cell exhaustion is low, suggesting that T cells are being functionally activated in the lungs of IL12-GEMy-treated mice. Single cell analysis demonstrates the upregulation of key T cell activation markers in cytotoxic T cells including Il2ra (CD25), Tnfrsf18 (GITR), Cd69 and Klrg1 (FIG. 3E). Pathway analysis of the cytotoxic T cell cluster from scRNA-seq supports these findings, as metabolic activity is enriched in these cells and T cell exhaustion signaling is downregulated (FIG. 3E). These data implicate cytotoxic T cells as key effectors of IL12-GEMy cell therapy on limiting metastatic progression.

As markers and gene expression pathways associated with T cell activation were upregulated in the lungs of IL12-GEMy treated mice, the mechanism of T cell activation by IL12-GEMys was investigated by analyzing the impact of IL12-GEMys on T cell function in vitro. Given the strong Ifng gene signature seen in the transcriptomic analysis of whole lung and the induction of Ifng in the cytotoxic T cell cluster by single cell analysis, the ability of IL12-GEMys to induce IFNγ production by T cells in an isolated culture system was evaluated. Co-culture of T cells with IL12-GEMys enhanced the ability of both naïve and activated OT-I CD8⁺ and OT-II CD4⁺ T cells to produce IFNγ in vitro in response to cognate peptide relative to co-culture with non-transduced myeloid cells (FIG. 3F-G). These data demonstrate the direct impact that IL12-GEMys have on T cell IFNγ production and support our previous findings that IL12-GEMys induce IFNγ production by T cells in vivo.

Although, there was no significant decrease in the total number of CD11b⁺ myeloid cells in the lungs of IL12-GEMy-treated mice compared to controls, there was a change in the composition of myeloid cell populations (FIG. 3H). There was a significantly higher number of both monocytic and conventional DCs (CD11b⁺CD11c⁺ and CD11b⁻ CD11c⁺, respectively), while there were no changes in number or frequency of monocyte (CD11b⁺Ly6C⁺Ly6G⁻) or macrophage (CD11b⁺CD43⁺Ly6C⁺F4/80⁺) populations (FIG. 3H). This indicates that IL12-GEMy treatment enhances the professional antigen-presenting DC population to activate adaptive immunity.

There were minimal changes in the number of myeloid populations in the spleen, lymph nodes, and tumor between IL12-GEMy-treated and control mice (FIG. 3I), apart from GEMy treatment significantly decreasing the frequency of overall myeloid cells in the tumor-draining lymph nodes (FIG. 3I).

IL12-GEMy cell number remained stable in the lung up to a week after transfer (FIG. 3D), indicating that the IL12-GEMys do not expand in vivo, which is a cause of toxicity associated with many T cell adoptive transfer therapies.

Overall, an increase of T cells with IL12-GEMy treatment without a dramatic change in total myeloid cell infiltrate was also observed by CODEX immunofluorescence imaging of lung tissue (FIG. 3J).

These data indicate that IL12-GEMy treatment reshapes the metastatic environment by promoting the accumulation of antitumor immune populations including activated T cells, NK cells and DCs.

### Example 4

This example demonstrates that IL-12-GEMys reverse the core immune suppression program in the pre-metastatic lung.

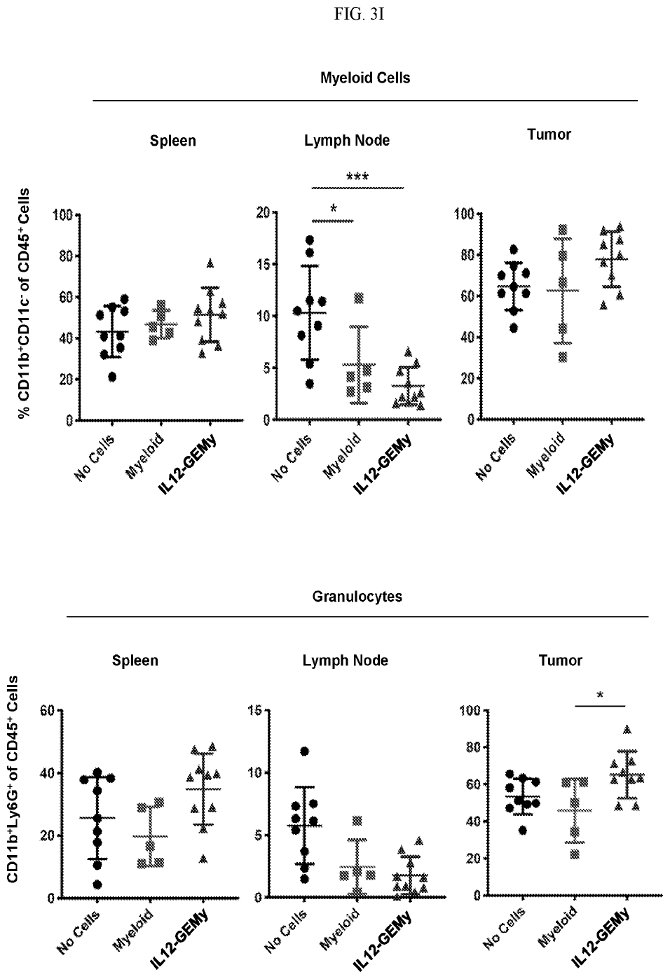

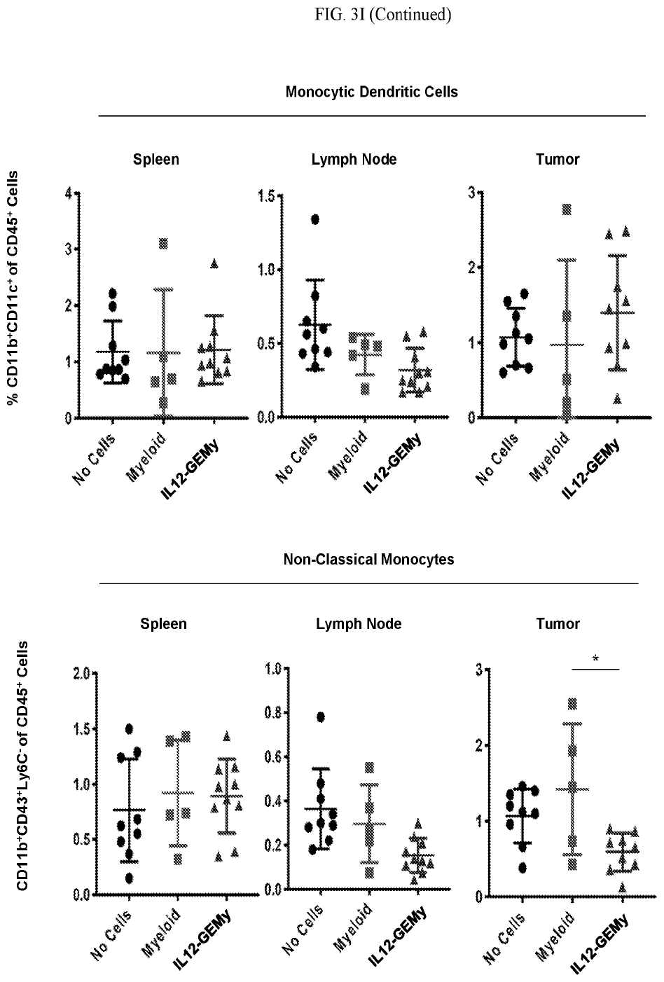

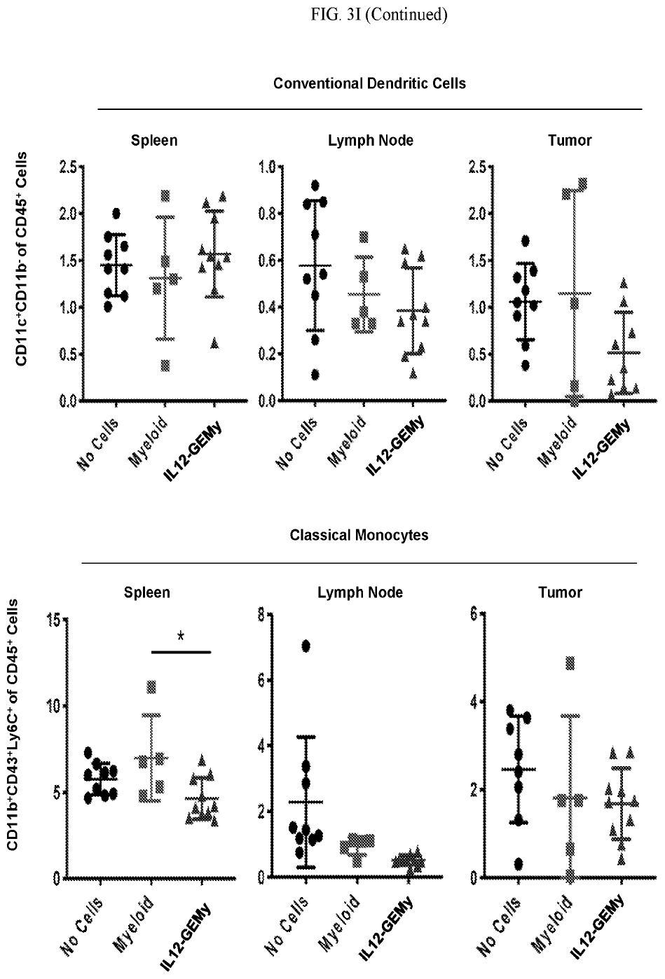

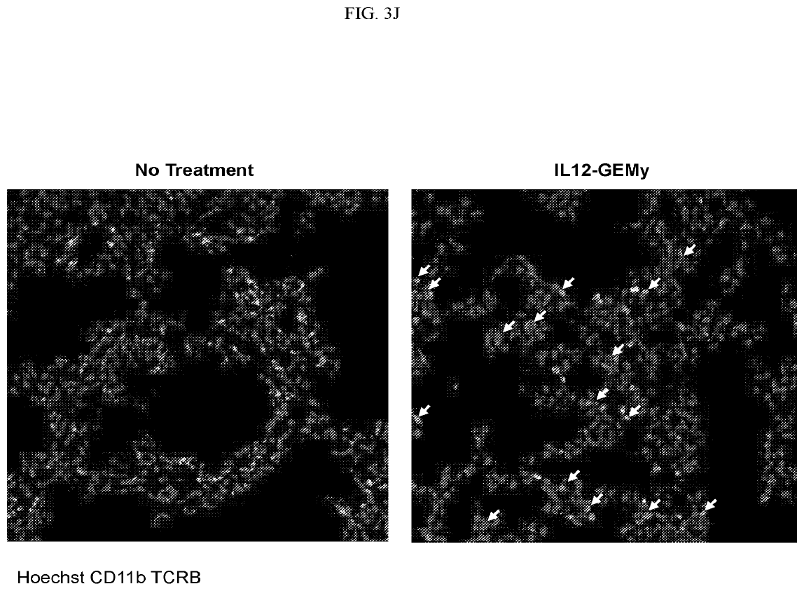

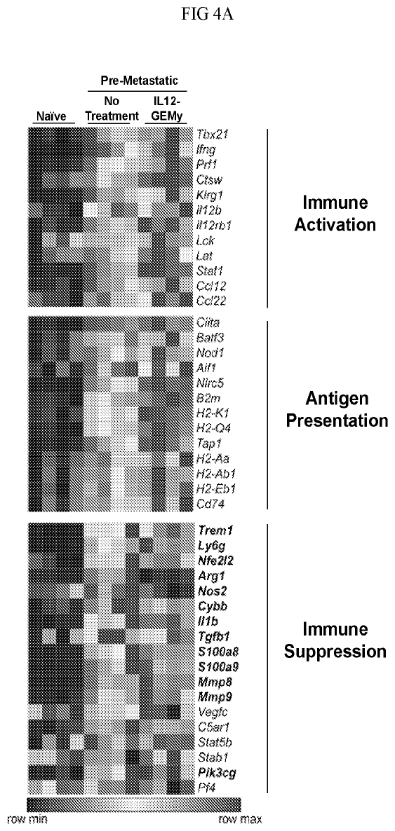

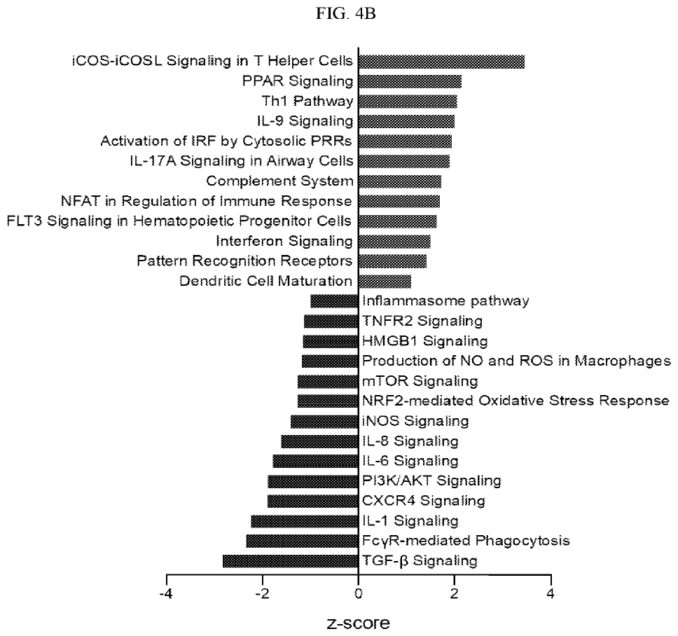

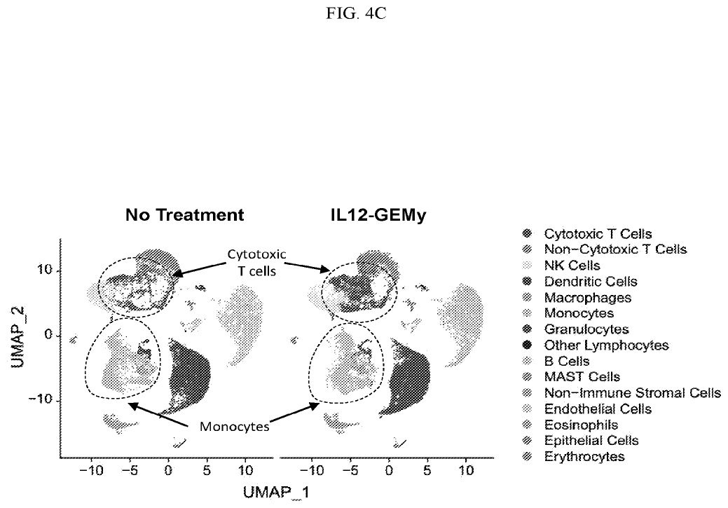

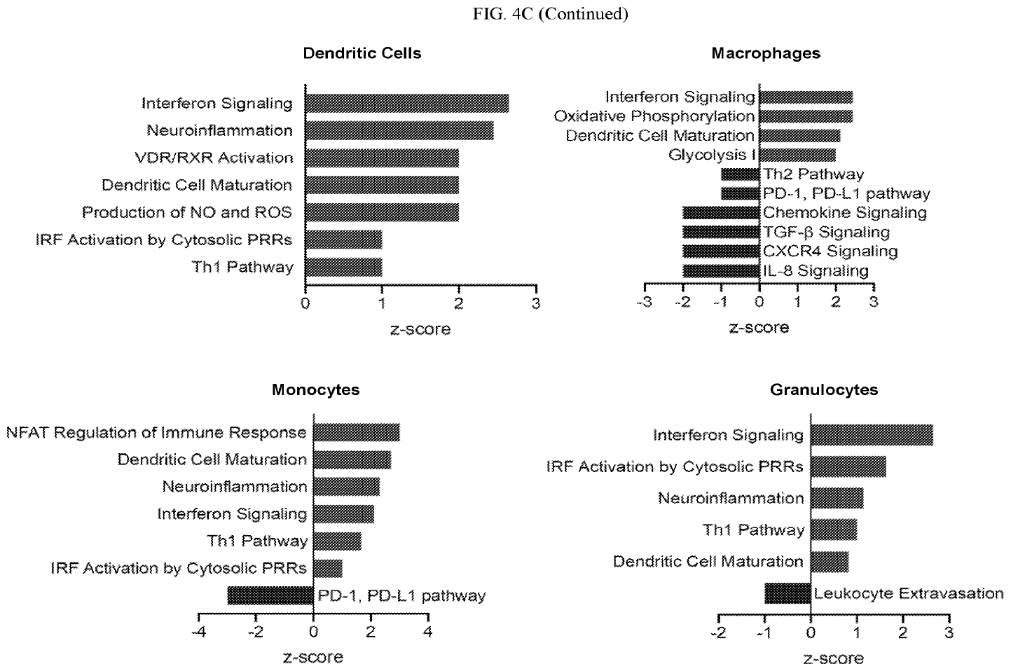

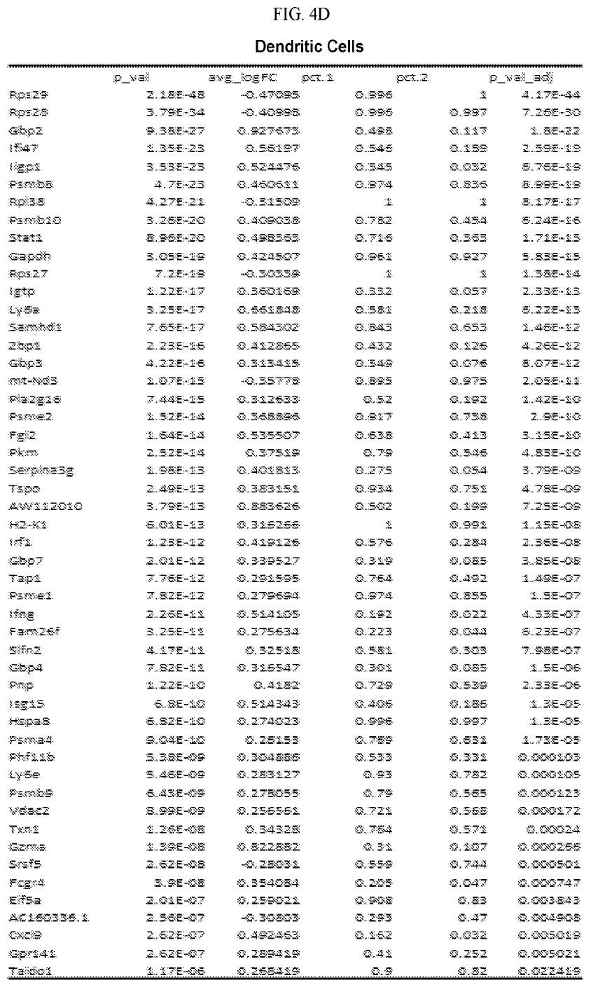

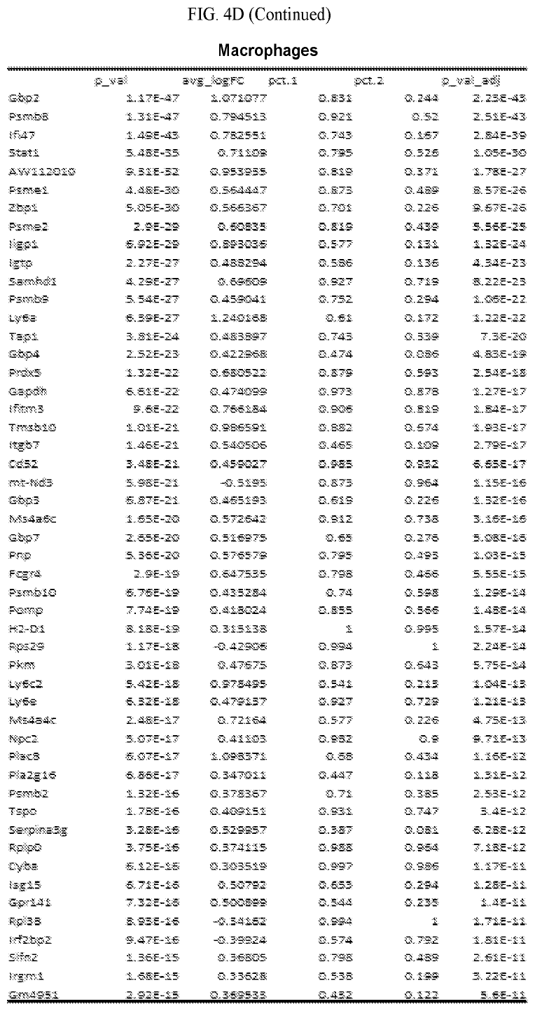

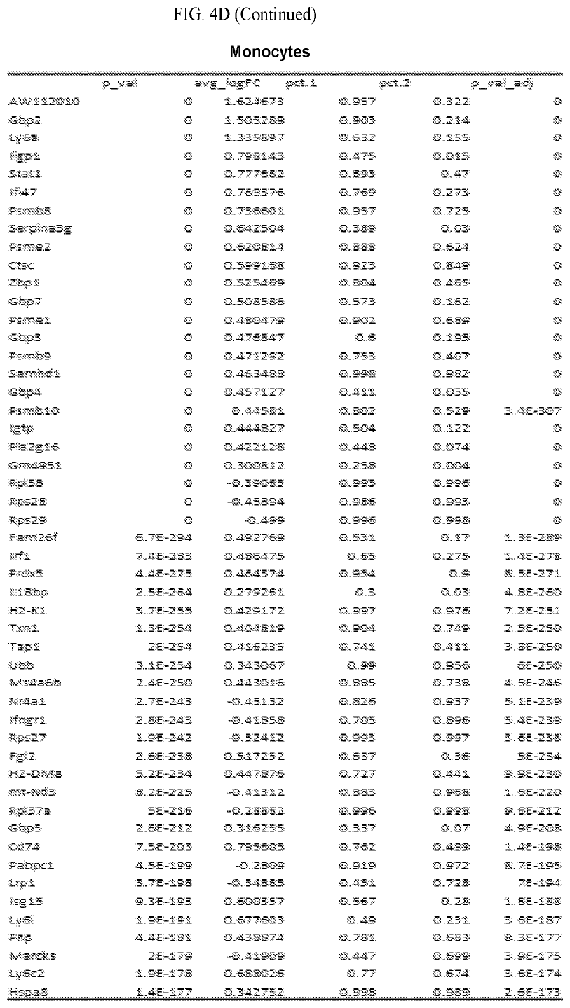

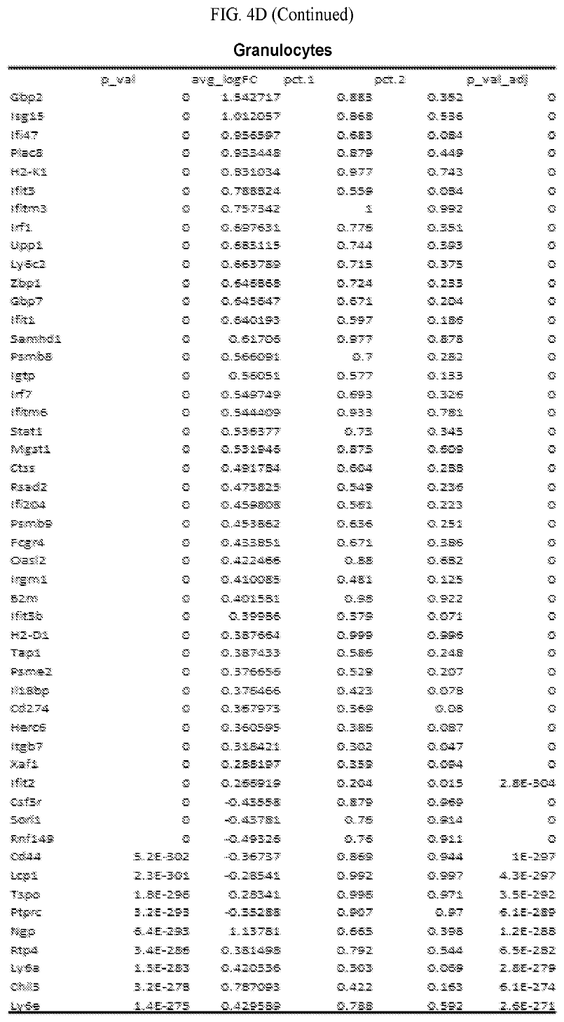

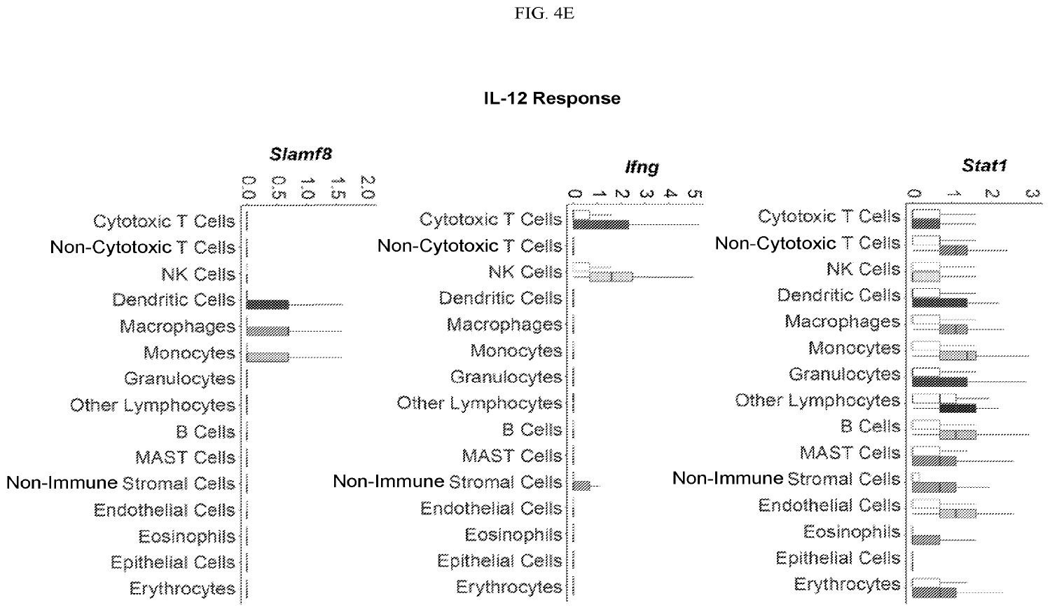

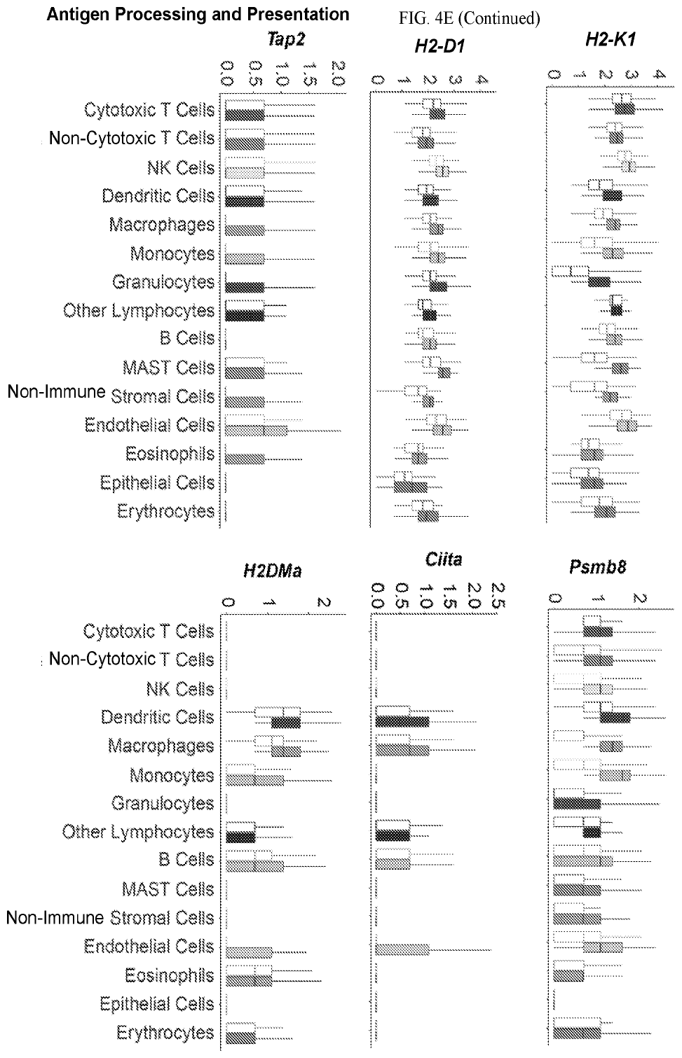

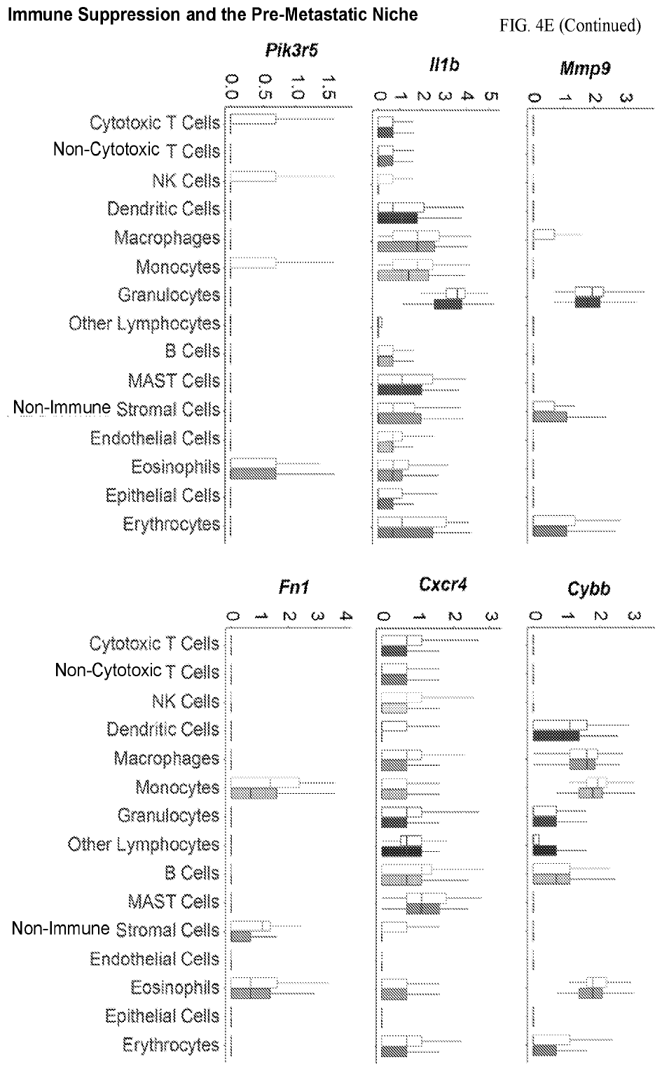

To determine the direct functional impact of IL12-GEMy treatment on the pre-metastatic microenvironment, the transcriptomic landscape of the pre-metastatic lungs of primary tumor-bearing mice three days after treatment with IL12-GEMys was profiled. IL12-GEMy treatment generates a global shift in the transcriptional program of pre-metastatic lungs that is distinct from the lungs of non-treated tumor-bearing mice. IL12-GEMy treatment induces genes associated with Th1 immune activation (Tbx21, Ifng, and Stat1) and cytotoxicity (Prf1, Ctsw), as well as antigen presentation (Ciita, Batf3, B2m, Tap1, H2-K1, H2-Q4, H2-Aa, H2-Ab1 and H2-Eb1) (FIG. 4A). Strikingly, immunosuppressive genes that we identified to be upregulated in pre-metastatic lungs as part of the core immune suppression program are downregulated with IL12-GEMy treatment (Trem1, Ly6g, Nfe2l2, Arg1, Nos2, Cybb, Il1b, Tgflb1, S100a8, S100a9, Mmp8, Mmp9, and Pik3cg) (FIG. 4A).

Pathway analysis revealed the upregulation of T cell pathways related to antitumor activity, such as ICOS signaling, PPAR signaling, Th1 activation, NFAT signaling, and interferon signaling, which are downstream pathways of IL-12 signaling and are associated with activation of adaptive immunity and antitumor responses (FIG. 4B). In parallel, the transcriptional profile of IL12-GEMy-treated lungs revealed a significant downregulation of pathways involved in niche-associated immune suppression such as TGF-3, IL-1, IL-6 and iNOS signaling, as well as oxidative stress response and the production of NO and ROS (FIG. 4B). This reversal of the core immune suppression gene niche program illustrates the plasticity of the pre-metastatic niche phenotype that can be targeted by the introduction of a small population of GEMys to induce profound reprogramming of the niche environment in the lungs of tumor-bearing mice.

In order to examine the transcriptional effects of IL12-GEMys on specific cell populations, scRNA-seq analysis on lungs from tumor-bearing not treated and IL12-GEMy-treated mice was performed. Although there was no significant change in the relative numbers of the myeloid cell populations, myeloid cells did demonstrate a population shift representative of profound transcriptional reprogramming in response to IL12-GEMy (FIG. 4C). To better understand IL12-GEMy-dependent phenotypic changes in the transcriptional programs of myeloid cells, pathway analysis of specific myeloid populations in the lungs of IL12-GEMy-treated versus non-treated tumor-bearing mice was performed (FIG. 4C). Marked activation of interferon and Th1 pathways was observed in myeloid populations (FIG. 4C). Moreover, gene pathways related to DC maturation were upregulated in monocyte, macrophage, and granulocyte cell clusters (FIG. 4C). Notably, there was a strong downregulation of pathways within myeloid subsets including the PD-1/PD-L1 axis, genes associated with Th2 signaling, TGF-β and IL-8 signaling, all associated with immunosuppressive myeloid biology (FIG. 4C). There was also a strong reduction in genes associated with leukocyte extravasation within the granulocyte population (FIG. 4C). The top 50 genes upregulated within each myeloid cell population could be used as a biomarker of response to GEMy therapy (FIG. 4D). These findings implicate IL12-GEMy cell therapy in reducing a multitude of diverse signaling pathways within the pre-metastatic niche that can contribute to disseminated tumor cell invasion, immune evasion and survival.

To further elucidate the contribution of specific cell types in response to IL12-GEMy treatment, the expression of various relevant genes per cluster was examined (FIG. 4E). One of the main downstream effects of IL-12 signaling is IFNγ production, which is robustly induced in cytotoxic T cells and NK cells in response to IL12-GEMy administration, and to a lesser extent in non-immune stromal cells. IFNγ signals through Stat1, which is highly upregulated by many cell types in the pre-metastatic niche in response to IL12-GEMys. IFNγ-induced Slamf8, a negative regulator of ROS production and migration in myeloid cells, is robustly upregulated in DCs, macrophages, and monocytes. IL-12 signaling is also associated with the upregulation of antigen processing and presentation. Many genes associated with both MHC class I (H2-K1, H2-D1, Psmb8, Tap2) and MHC class II (Ciita, H2-DMa) antigen processing and presentation are dramatically upregulated in myeloid cells as well as on many other cell types. Furthermore, single cell analysis revealed that the expression of many of the immunosuppressive genes is reduced in specific cell populations, such as Mmp9 in macrophages, Il1b in NK cells and all myeloid populations and Cybb in DCs, macrophages and monocytes. Additionally, Pik3r5, a recently identified molecular switch that controls immune suppression, is also reduced in cytotoxic T cell, NK cell and monocyte populations. Other genes that play vital roles in the pre-metastatic niche are also differentially regulated. Cxcr4, an important chemokine for hematopoietic stem cell homing, is reduced in many cell types. Additionally, perivascular cell-derived fibronectin (Fn1) is a key component of the pre-metastatic niche. Here, its demonstrated that monocytes and eosinophils also contribute to fibronectin production, which is decreased in the presence of IL12-GEMys.

Together, these data provide evidence that IL12-GEMys have the ability to induce a cascade of transcriptional events in multiple cell types to reverse immune suppression in the pre-metastatic lung microenvironment, leading to the activation of adaptive T cell immune responses. Further, it offers novel insight into the cell-specific responses to IL-12 and IFNγ signaling in vivo, demonstrating both shared and exclusive cell-specific responses orchestrated within the niche environment to generate the collective antitumor phenotype.

### Example 5

This example demonstrates that treatment with IL12-GEMys limits primary tumor burden and metastatic progression in syngeneic tumor models.

To determine whether reversing the core immune suppression signature in the pre-metastatic microenvironment with IL12-GEMy treatment has a therapeutic impact on tumor progression, 8×10⁶ vector control or IL-12 GEMys were transferred into tumor-bearing mice intravenously on day 12. IL12-GEMys lead to the regression of large established primary rhabdomyosarcoma tumors and significantly improved survival, doubling the mean survival time from 22 to 45.5 days (FIG. 5A). Additionally, non-transduced myeloid cells, in vitro IL-12 treated non-transduced myeloid cells (IL-12 pre-treated), or IL12-GEMys were adoptively-transferred at low doses (s 5×10⁶ cells) into mice bearing M3-9-M primary tumors on days 12, 19, and 26 post tumor inoculation (FIG. 5B-5C). Only IL12-GEMys reduced tumor progression and significantly increased the survival of tumor-bearing mice compared to mice receiving non-transduced myeloid cells, IL-12 pre-treated myeloid cells, or no treatment (FIG. 5B).

Given the flow cytometric and transcriptomic data that demonstrates the profound effect that IL12-GEMy treatment has on the metastatic lung microenvironment, the impact of IL12-GEMy treatment on metastasis was evaluated. The lungs of mice bearing primary luciferase-expressing M3-9-M tumors were evaluated by bioluminescent imaging for quantification of metastatic tumor burden. Lungs from IL12-GEMy-treated mice had significantly less metastatic burden and frequency of metastasis compared to lungs from non-treated mice (FIG. 5C). By contrast, there was no significant difference in metastatic burden nor metastatic frequency in mice that received either non-transduced or IL-12 pre-treated myeloid cells compared to mice that received no treatment. Together, these results demonstrate that IL12-GEMy therapy is capable of significantly reducing spontaneous metastasis in mice.

To determine whether the efficacy of IL12-GEMy treatment could be improved by increasing the dose, the dose-dependent impact of IL12-GEMys was examined by administering either a single high dose (8×10⁶ IL12-GEMys), a single low dose (1×10⁶ IL12-GEMys), or no treatment to mice with established M3-9-M primary tumors (FIG. 5D). Mice that received a high dose of IL12-GEMys had delayed primary tumor growth and significantly improved survival compared to non-treated and low dose IL12-GEMy-treated mice, indicating that the therapeutic efficacy of IL12-GEMys is dose-dependent.

To test if the efficacy of IL12-GEMy therapy is enhanced by cyclophosphamide chemotherapy pre-conditioning, M3-9-M tumor-bearing mice were administered a single dose of 2 mg cyclophosphamide ten days post tumor inoculation followed by IL12-GEMy treatment two days later (FIG. 5E). Cyclophosphamide alone had a modest delay in primary tumor growth and significantly increased survival compared to non-treated mice. The combination of cyclophosphamide with either low dose or high dose IL12-GEMys demonstrated a marked decrease in tumor progression and significantly improved survival compared to cyclophosphamide alone. This indicates that IL12-GEMy treatment is more efficacious when used in combination with a single-dose cyclophosphamide pre-conditioning regimen. Furthermore, cyclophosphamide combined with IL12-GEMy treatment was curative in 30% of mice receiving a low dose of IL12-GEMys but reached 66.7% in mice that received a high dose of IL12-GEMys. These data indicate that the therapeutic benefit of chemotherapy pre-conditioning may be generalized to many types of cell-based immunotherapies and could have greater implications for how IL12-GEMys might be used in the clinical setting in the future.

The data demonstrate that IL12-GEMy-based therapy limits spontaneous metastatic progression from a primary tumor (FIG. 5C). To evaluate the impact of IL12-GEMys on overt metastatic tumors, albino C57BL/6 mice were given M3-9-M i.v. to establish lung lesions followed by IL12-GEMy treatment seven days later and assessed for metastatic progression and survival by bioluminescence imaging (FIG. 5F). Mice treated with IL12-GEMys had decreased metastatic burden and increased survival, indicating that IL12-GEMys are capable of alleviating bulk metastatic tumor growth.

Clinically, many rhabdomyosarcoma patients undergo surgical resection of their primary tumor; however, 30% of patients later relapse with metastatic disease. In order to model neo-adjuvant treatment, mice were treated with IL12-GEMys on day 17 followed by amputation of the tumor-bearing leg on day 24. IL12-GEMy cell therapy significantly extended the survival of mice in the neo-adjuvant setting (FIG. 5G). Together, these data indicate that IL12-GEMy therapy can target metastasis and has efficacy in the clinically-relevant neo-adjuvant setting.

Given that IL12-GEMys efficiently home to the liver and to extend the findings to an epithelial tumor model, the impact of IL12-GEMy therapy on cancer progression was tested in a highly-aggressive pancreatic cancer liver metastasis model. KPC177669 is a Kras⁻/p53⁻ tumor cell line derived from the KPC mouse model that metastasizes specifically to the liver when cells are delivered into splenic circulation. Administration of IL12-GEMys five days post intrasplenic injection of tumor cells delayed primary tumor and metastatic outgrowth and significantly extended the survival of mice with KPC177669 pancreatic tumors, with some IL12-GEMy-treated mice never developing detectable tumor lesions (FIG. 5H).

To determine that the effects of GEMy treatment are not specific to the M3-9-M model, BALB/c mice bearing 4T1 mammary carcinoma were treated with BALB/c-derived GEMys 12 days post-tumor inoculation and the mice were monitored for primary tumor growth and survival (FIG. 5I). As expected, GEMy therapy reduced primary tumor growth and significantly extended the survival time of 4T1 tumor-bearing mice.

Together, these data demonstrate, for the first time, the marked therapeutic efficacy of the i.v. administration of a myeloid-based immunotherapy for the treatment of tumor progression and metastasis in multiple metastatic tumor models.

### Example 6

This example demonstrates that IL12 GEMy when combined with cyclophosphamide and fludarabine conditioning cures rhabdomyosarcoma tumor bearing mic with advanced disease.

To determine the efficacy of IL12-GEMys in combination with chemotherapy conditioning with fludarabine/cyclophosphamide in metastatic rhabdomyosarcoma model. In the clinical setting most adoptive cell therapy is delivered after chemotherapy conditioning with cyclophosphamide and fludarabine. Mice were injected with M3-9-M-OVA and then on day 8 after tumor implantation were treated with 2 mg of cyclophosphamide and 5 mg of fludarabine i.p. and immune cell populations. Mice were injected with M3-9-M-OVA and treated with 2 mg of cyclophosphamide and 5 mg of fludarabine i.p. on day 8 and on day 10 were given 5×10⁶ IL-12 GEMys or no cells. Surprisingly, all mice given IL12 GEMys after fludarabine/cyclophosphamide were cured.

In order to examine for the development of long term anti-tumor immune response in cured mice, IL12-GEMy-cured mice were re-challenged with unlabeled M3-9-M cells or their original tumor cell line, M3-9-M-ffLuc2-mCherry-OVA, in the contralateral leg compared to naïve age-matched controls. The IL12-GEMy cured mice did not develop a tumor when rechallenged with the same M3-9-M OVA tumor suggesting effective immunity. Interestingly the IL-12 GEMy cured mice that were given unlabeled M3-9-M cells had significantly delayed tumor growth compared to mice that were not prior cured which demonstrates development of T cell clones to more than the major OVA antigen and effective epitope spreading.

### Example 7

This example demonstrates that IL12-GEMy function is dependent on CD8⁺ T cells.

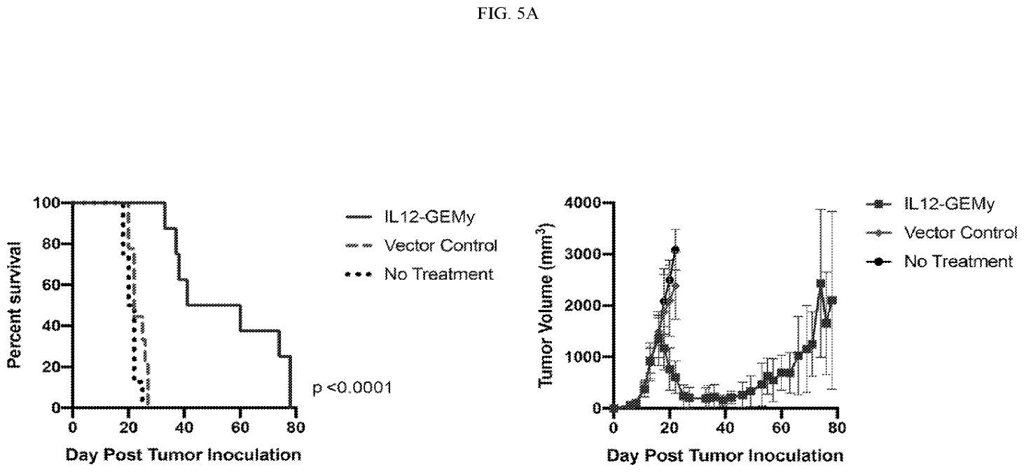

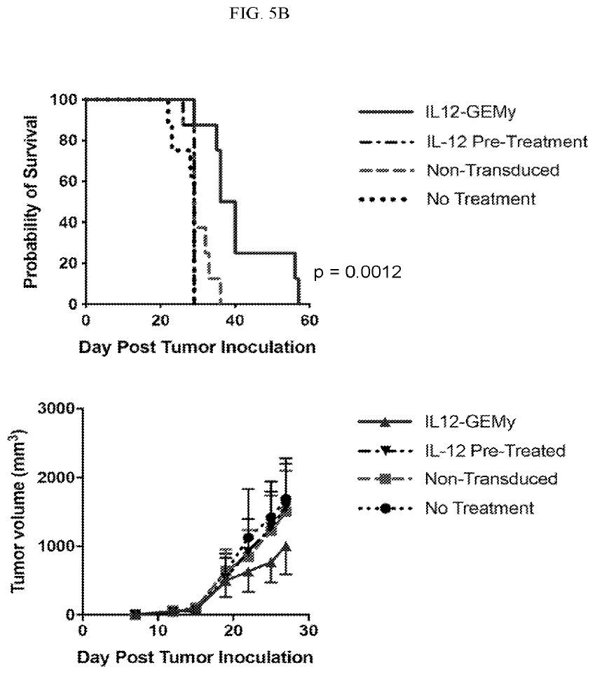

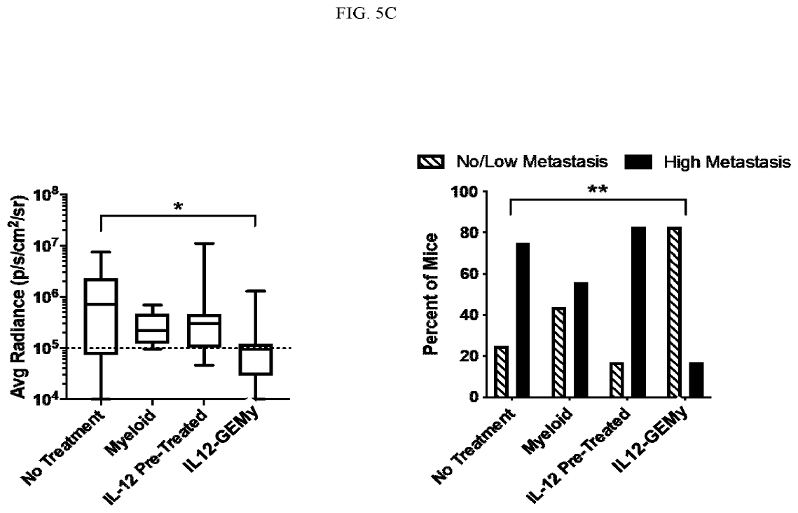

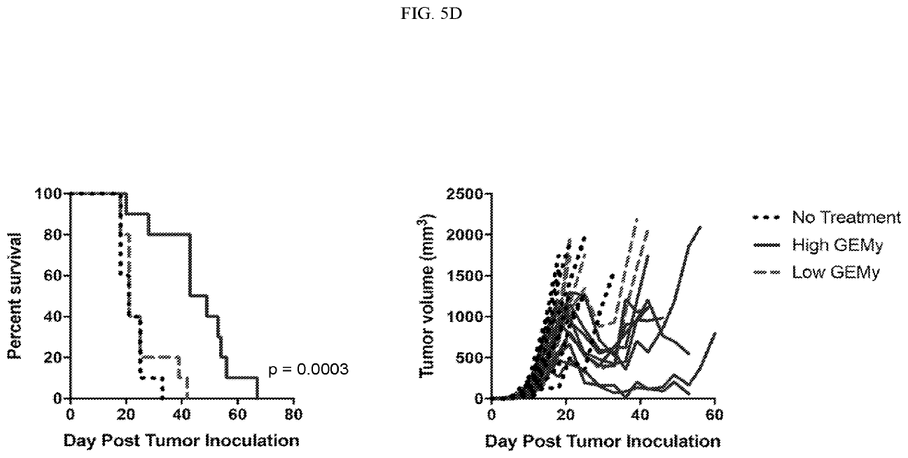

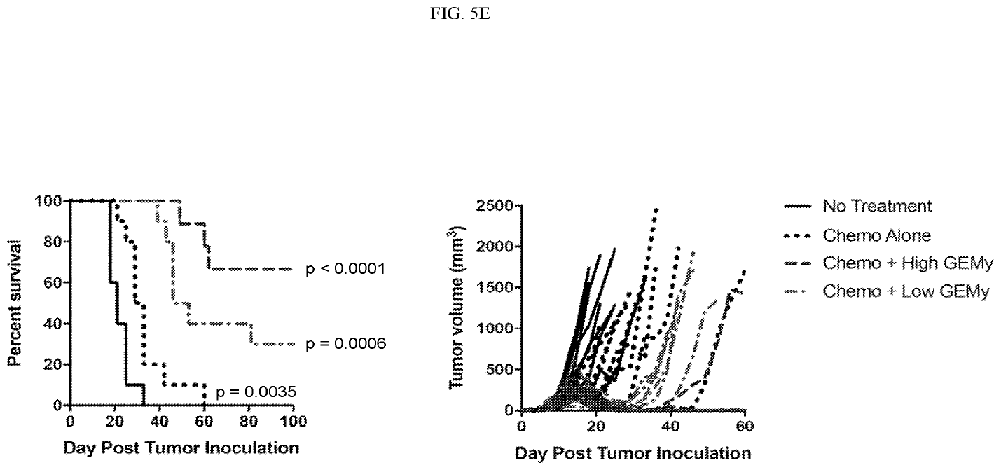

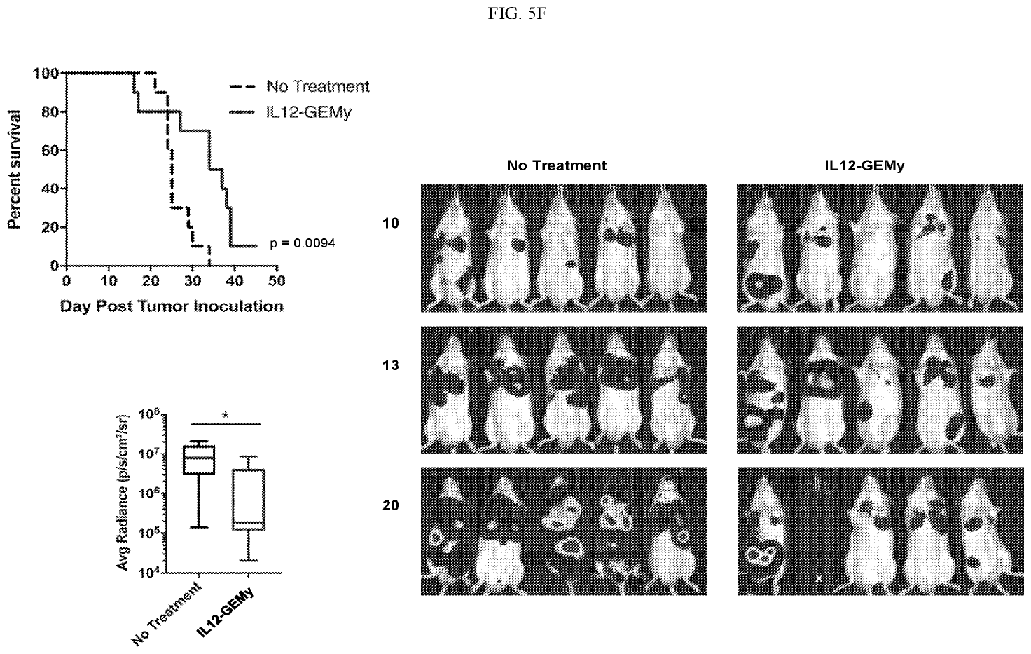

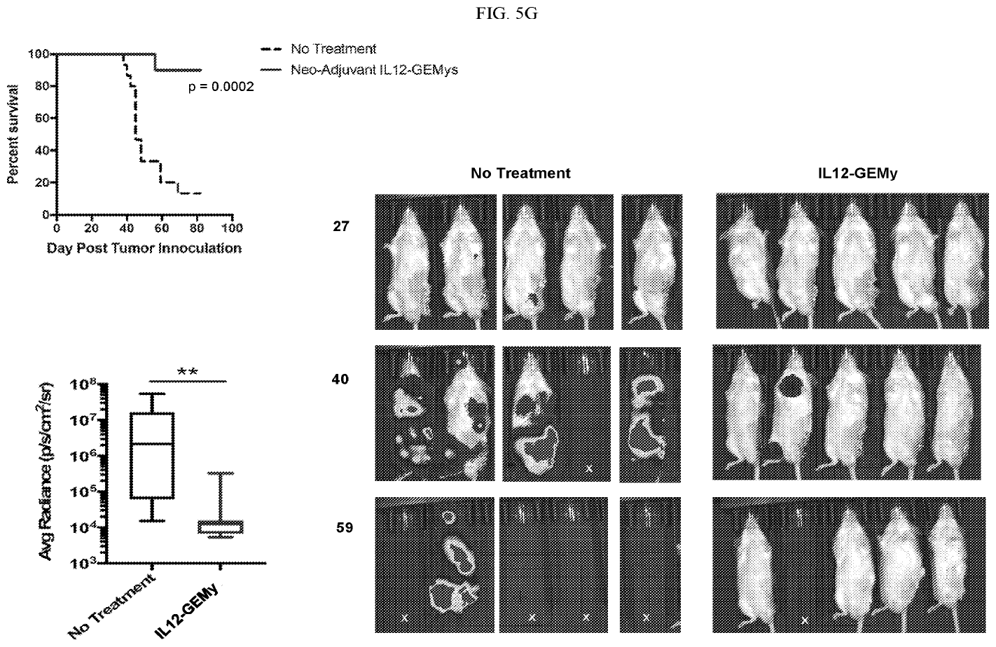

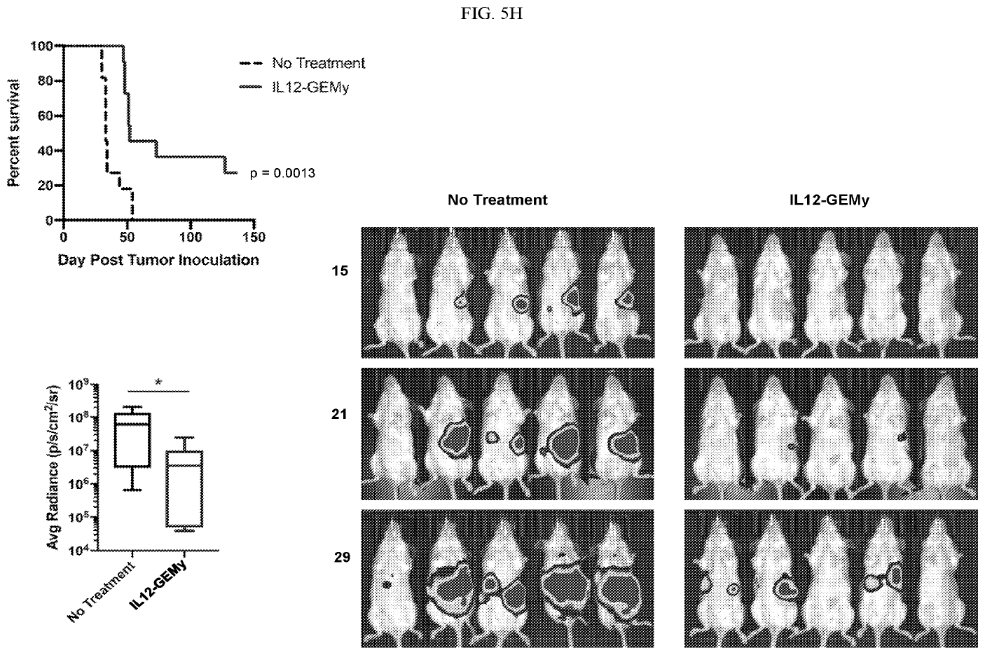

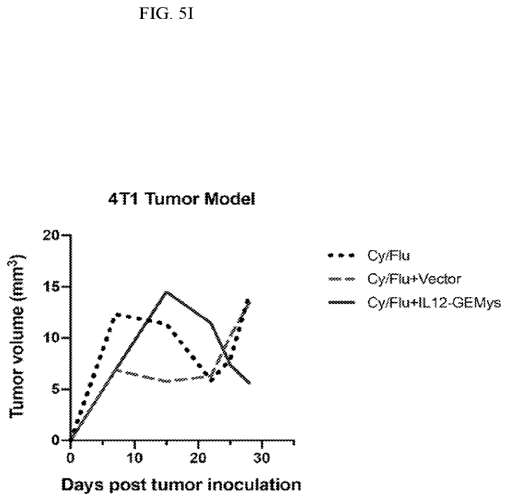

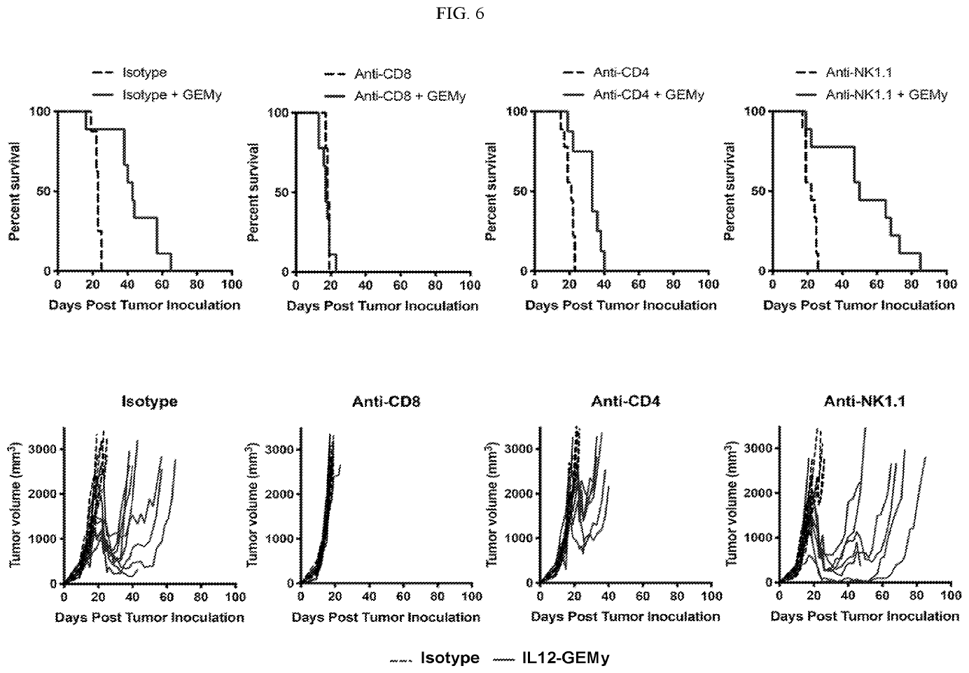

In vivo antibody depletion experiments were performed to target CD8⁺, CD4⁺, or NK1.1⁺ cells to evaluate which lymphoid populations are necessary for mediating the efficacy of IL12-GEMy therapy (FIG. 6). IL12-GEMy treatment in the isotype-treated group reduced large established primary tumors and significantly extended survival (p=0.008) of tumor-bearing mice (FIG. 6). Mice that were depleted of CD8⁺ cells had no response to IL12-GEMy therapy, indicating that CD8⁺ cells are essential for the antitumor effect of IL12-GEMys. CD4 targeting had a partial effect on GEMy efficacy based on survival data. In contrast, incomplete depletion of NK1.1⁺ cells in combination with IL12-GEMy treatment did not have a significant difference in survival compared to the isotype plus IL12-GEMy treatment. These data demonstrate that T cells play a critical role in the mechanism of action of IL12-GEMy therapy.

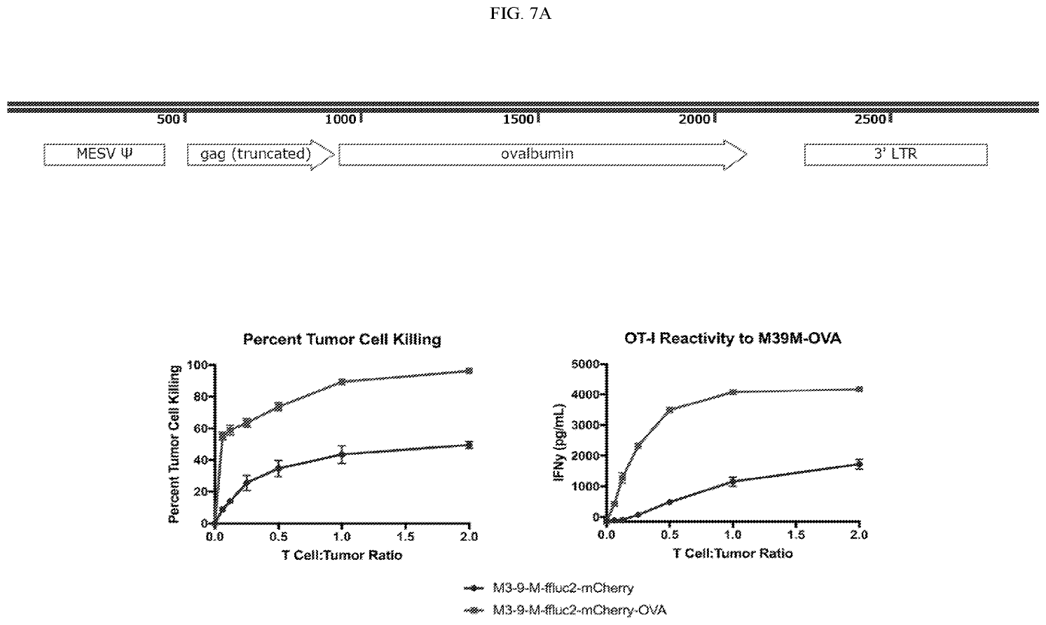

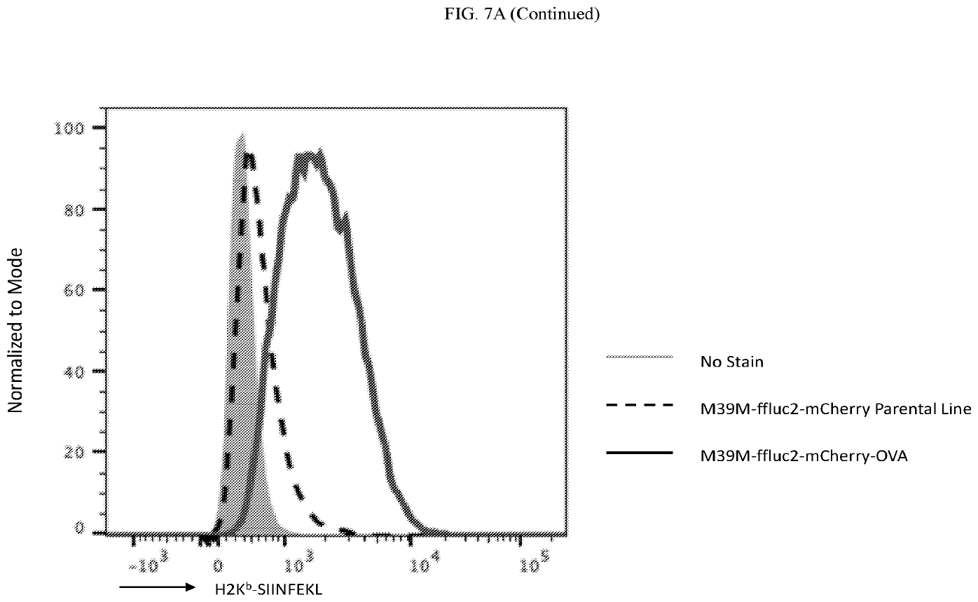

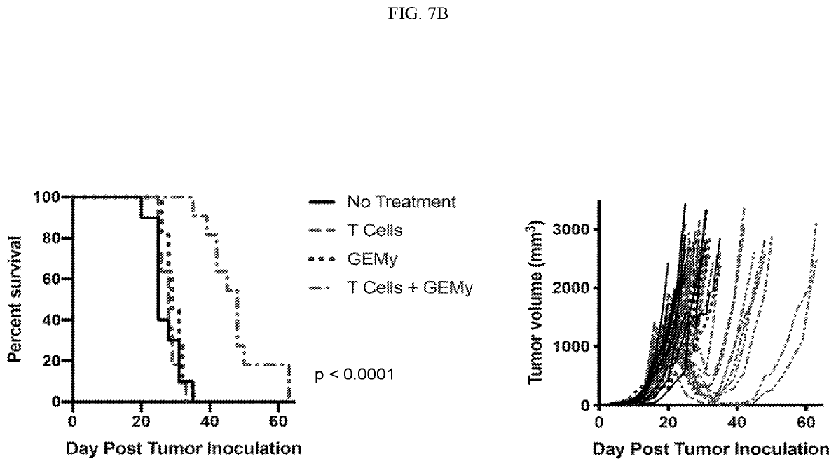

To better understand the mechanism of T cell activation by IL12-GEMys, the impact of IL12-GEMys on T cells in culture was tested. An OVA-expressing M3-9-M cell line that is sensitive to killing by OT-I T cells was generated (FIG. 7A). IL12-GEMys enhance the production of IFNγ by naïve and re-activated CD4+ and CD8+ cells (FIG. 7A).

IL12-GEMy therapy is dependent on CD8+ T cells and promoted IFNγ production of both naïve and reactivated CD4+ and CD8+ T cells in vitro (FIG. 3F-G) and in vivo (FIG. 4A, FIG. 4E). To determine if IL12-GEMy therapy can increase the efficacy of adoptive T cell therapy, an OVA-specific T cell system was used as a model for effective TCR based immunotherapy. OT-I T cells, IL12-GEMys, or a combination were transferred into M3-9-M-OVA tumor-bearing mice with subtherapeutic doses of IL12-GEMys and T cells (FIG. 7B). The transfer of T cells or low-dose IL12-GEMys alone did not impact primary tumor growth. IL12-GEMy treatment in combination with T cells was able to significantly increase the survival of the mice and lead to tumor regression. This enhancement of therapeutic activity was elicited in the absence of any preconditioning regimen, suggesting that the reversal of immune evasion with IL12-GEMy cell therapy alone was sufficient to enhance the antitumor activity of transferred T cells.

Together, these data indicate that IL12-GEMy therapy requires CD8+ T cells and is partially dependent on CD4+ T cells. Additionally, these data support that IL12-GEMys can enhance adoptive T cell therapy.

### Example 8

This example demonstrates that chemotherapy pre-conditioning in combination with IL12-GEMy therapy enhances adoptive T cell therapy and generates functional T cell memory.

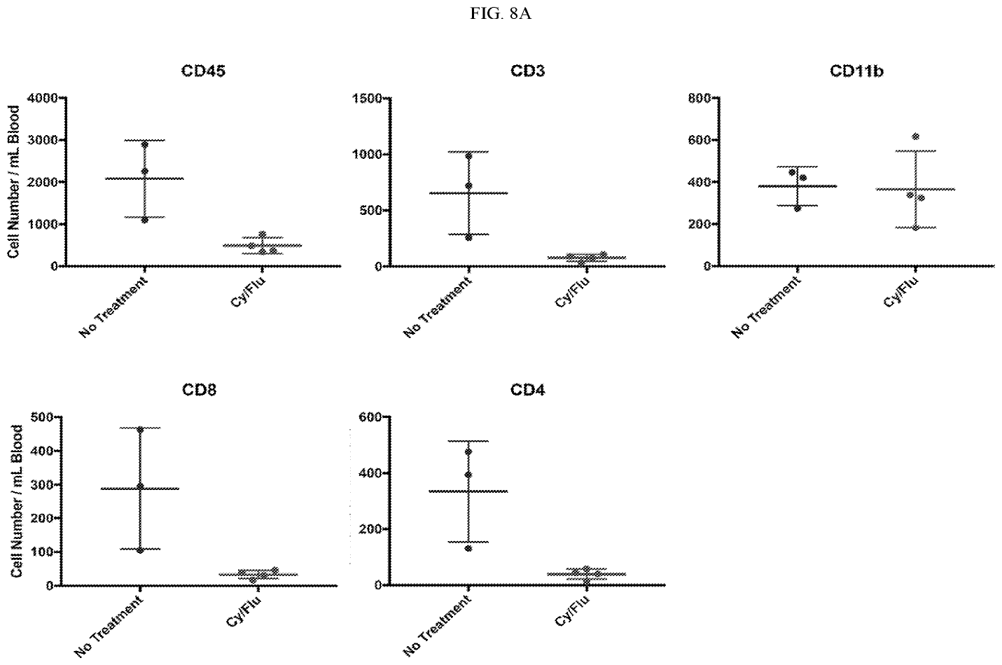

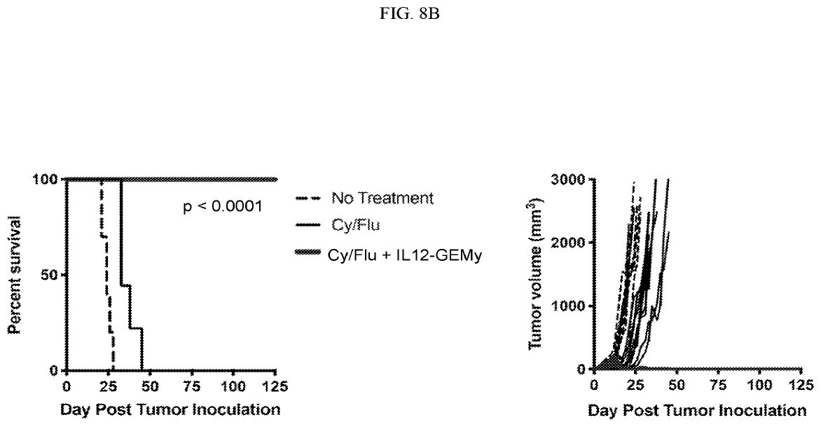

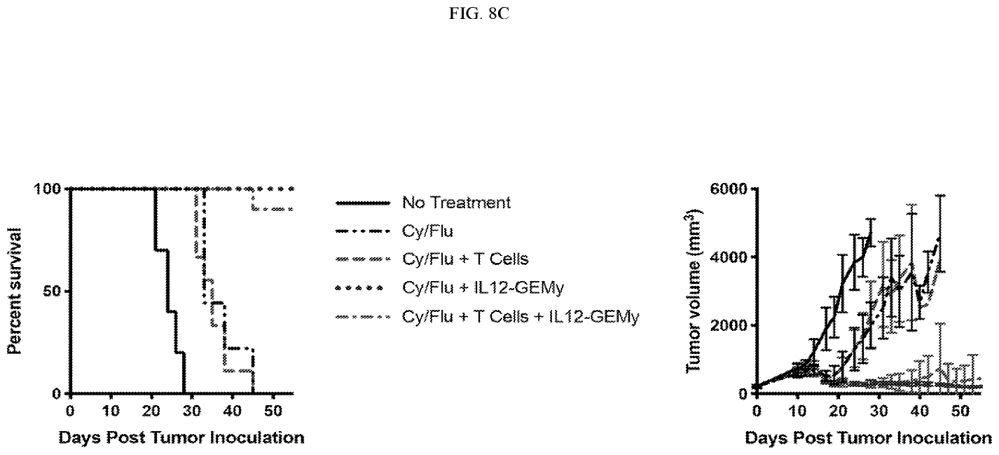

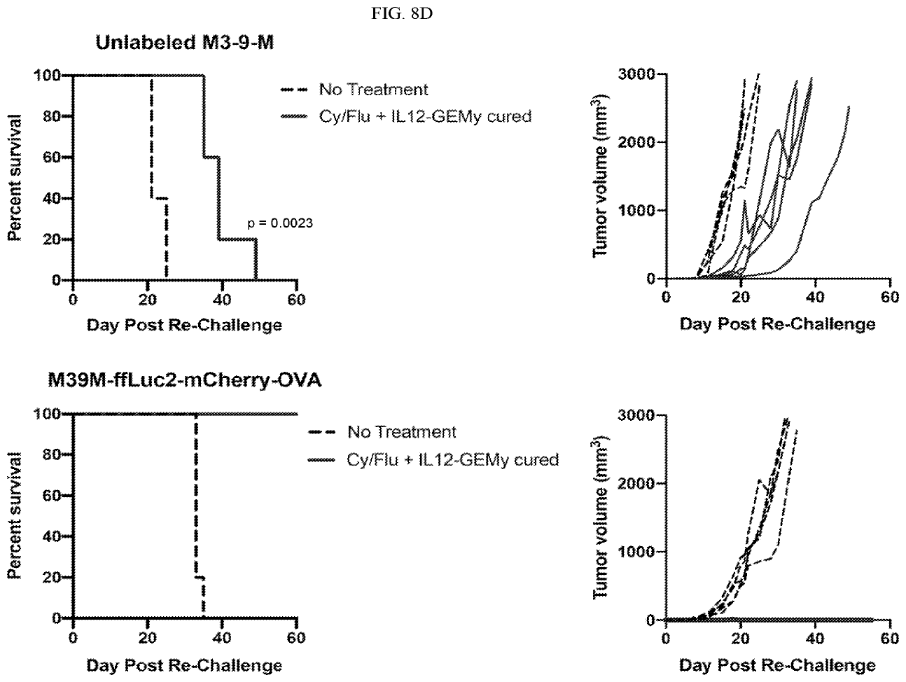

Most T cell therapies currently used in the clinic are given following a pre-conditioning regimen of cyclophosphamide and fludarabine (Cy/Flu). Depletion of circulating T cells was observed following Cy/Flu pre-conditioning while myeloid populations were not impacted in circulation at the time point examined (FIG. 8A). Unexpectedly, when combined with a single-dose pre-conditioning regimen of Cy/Flu forty-eight hours before cell transfer, IL12-GEMy cell therapy resulted in complete and durable cures of mice with established primary tumors (FIG. 8B). 100% of the mice receiving IL12-GEMys after Cy/Flu preconditioning were cured (n=10, experiment repeated twice).

In order to interrogate IL12-GEMy function in eliciting a complete immune response, cured mice were re-challenged with either unlabeled tumor or the original OVA-expressing tumor in the contralateral leg. When re-challenged with unlabeled M3-9-M, lacking the strong OVA antigen, IL12-GEMy-cured mice displayed a statistically-significant delay in tumor growth relative to age-matched naïve controls, evidence that IL12-GEMys elicit endogenous T cell responses that recognize multiple tumor antigens, including non-dominant antigens (FIG. 8C). Mice re-challenged with the original tumor line, M3-9-M-ffLuc2-mCherry-OVA, were immune over 100 days post-IL12-GEMy treatment, consistent with the generation of functional memory T cells (FIG. 8C).

Together, these studies demonstrate that IL12-GEMys support the function of tumor-specific CD8⁺ T cells by enhancing suboptimal T cell therapy, and that IL12-GEMy therapy is capable of generating endogenous long-lived T cell memory to multiple antigens. This stimulation of adaptive immune responses coupled with the reversal of the immune suppression program has a profound impact on tumor and metastatic progression in our preclinical models.

### Example 9

This example demonstrates the feasibility of generating human GEMys.

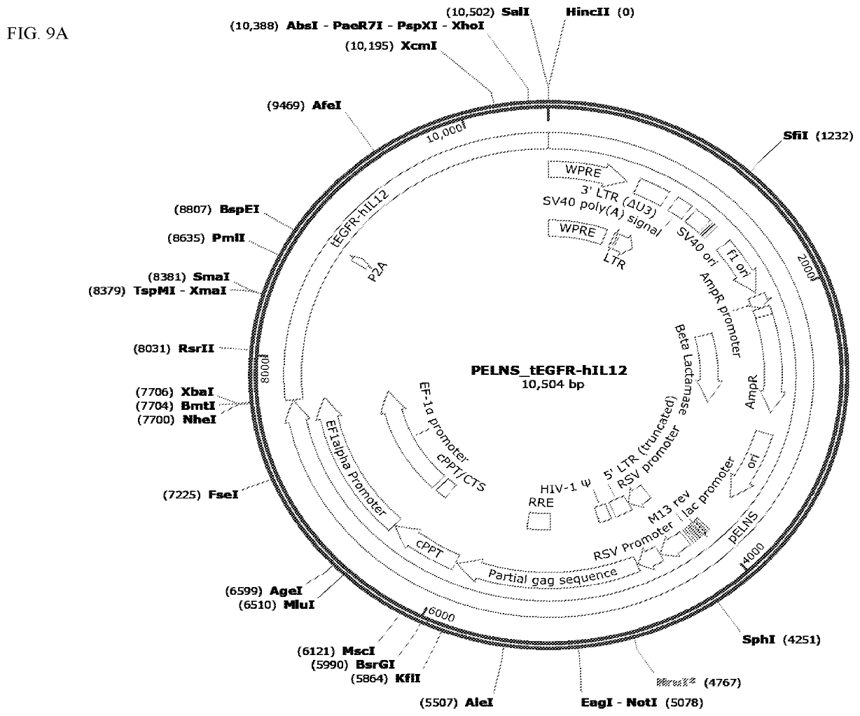

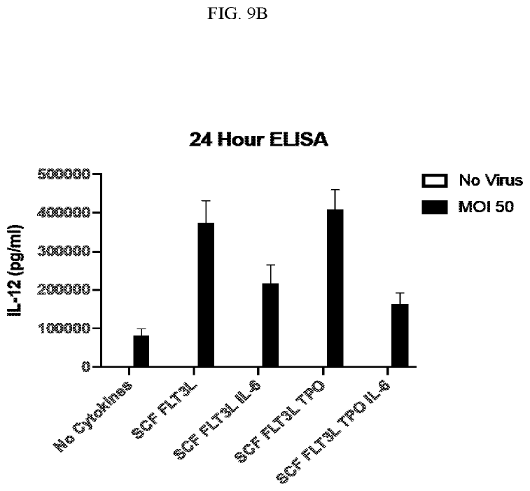

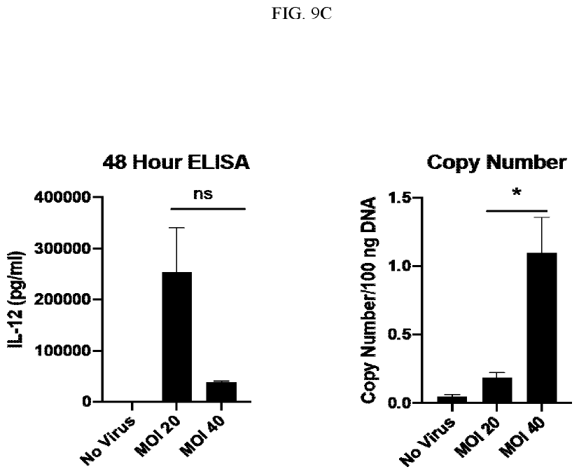

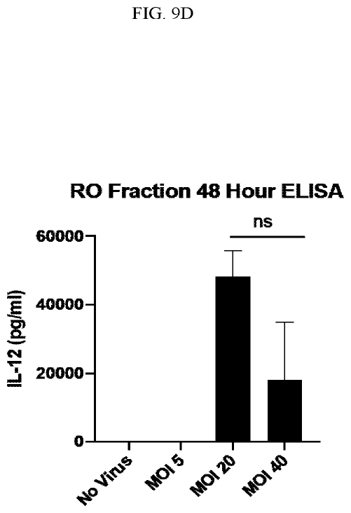

A human vector for generating human IL12-GEMys is depicted in FIG. 9A. Truncated EGFR (tEGFR) is used as a reporter to measure transduction efficiency and as a potential safety switch to deplete transduced cells in vivo by using anti-EGFR antibody (such as Cetuximab).

Human CD34+ stem cells were isolated from apheresis product, cultured under various cytokine conditions and transduced with various MOIs with the IL-12 expressing lentivirus. Human IL-12 was measured in the supernatant at 24 hours after transduction by ELISA (FIG. 9B).

Transduced human CD34+ cells were analyzed for IL-12 production by ELISA and DNA was isolated from the cells for copy number analysis by quantitative PCR (FIG. 9C).

Human monocytes from the RO fraction of elutriated apheresis product were transduced with lentivirus and culture supernatant was analyzed for IL-12 by ELISA.

These data show that the production of human GEMys expressing high levels of IL-12 is achievable from both CD34+ stem cells as well as peripheral blood monocytes.

### Example 10

This example demonstrates the ability to produce GEMys expressing different transgenes as well as more than one transgene at one time to orchestrate therapeutic responses (bi-GEMys).

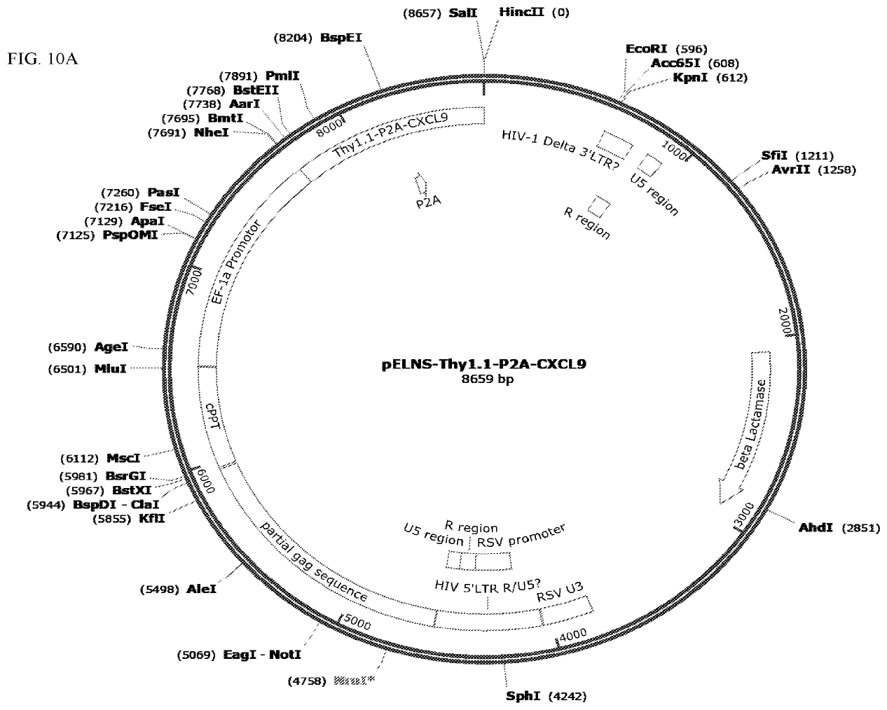

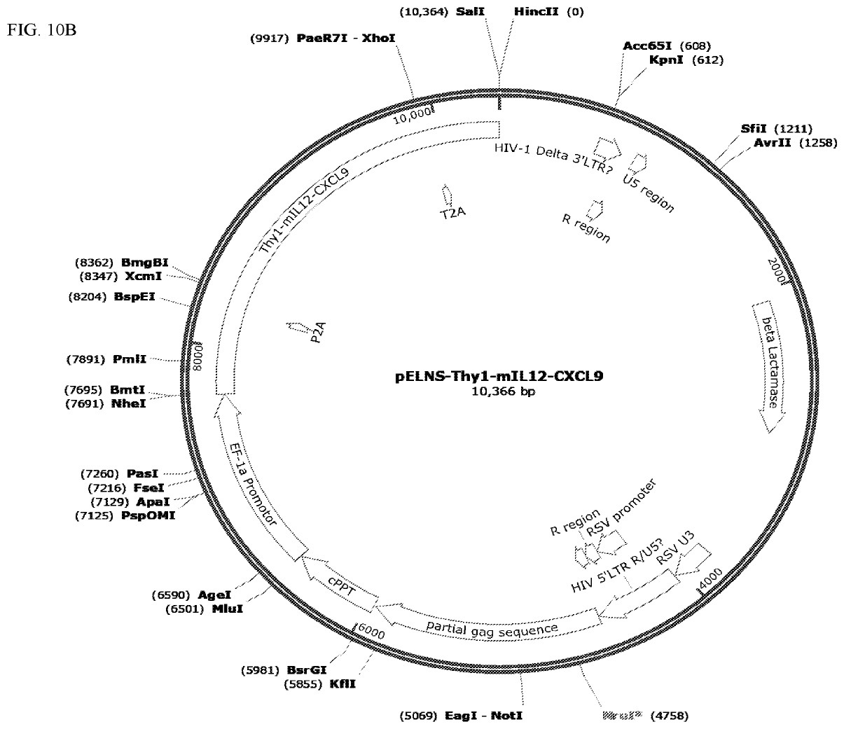

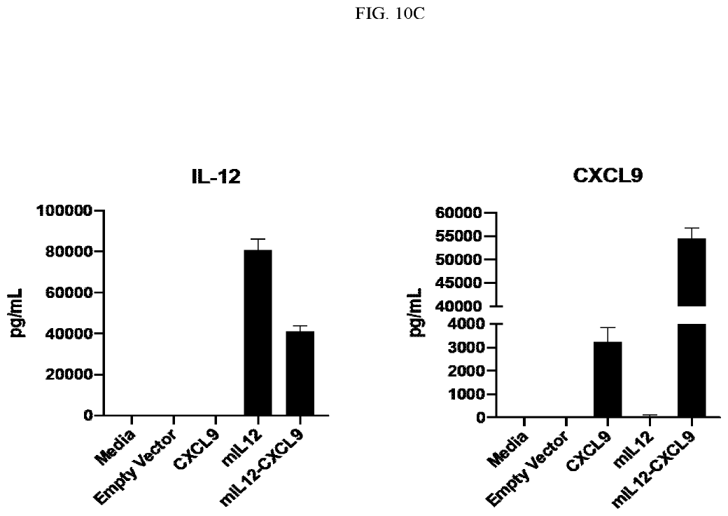

Lentiviral vectors were designed to express the T cell chemoattractant, CXCL9 (FIG. 10A), as well as IL-12 in combination with CXCL9 (FIG. 10B).

Mouse GEMys were generated as previously described and transduced with lentivirus to express IL-12, CXCL9, or IL-12 and CXCL9 together. Analysis of culture supernatant by ELISA demonstrates that both the IL12-GEMys and bi-GEMys produce IL-12, and CXCL9 GEMys as well and bi-GEMys produce CXCL9 (FIG. 10C).

These data provide evidence for the use of GEMys as a platform capable of expressing diverse cargo as well as their ability to express multiple proteins simultaneously.

### Example 11

This example demonstrates an exemplary protocol for the isolation of MSCs.

Growth in hypoxia (5% 02) slightly decreased CD45/TER119+ cells and increases CD105+ cells when MSCs are grown in MesenCult media (no supplements). Hypoxia also slightly increased CD105+ cells when MSCs are grown in MesenPure media. However, there was no difference in CD45/TER119 expression (˜99% of cells are CD45-TER119-regardless of normoxia or hypoxia). Hypoxia is thought to maintain the stem like phenotype and is likely most important for the initial growth/establishment of mouse derived MSCs.

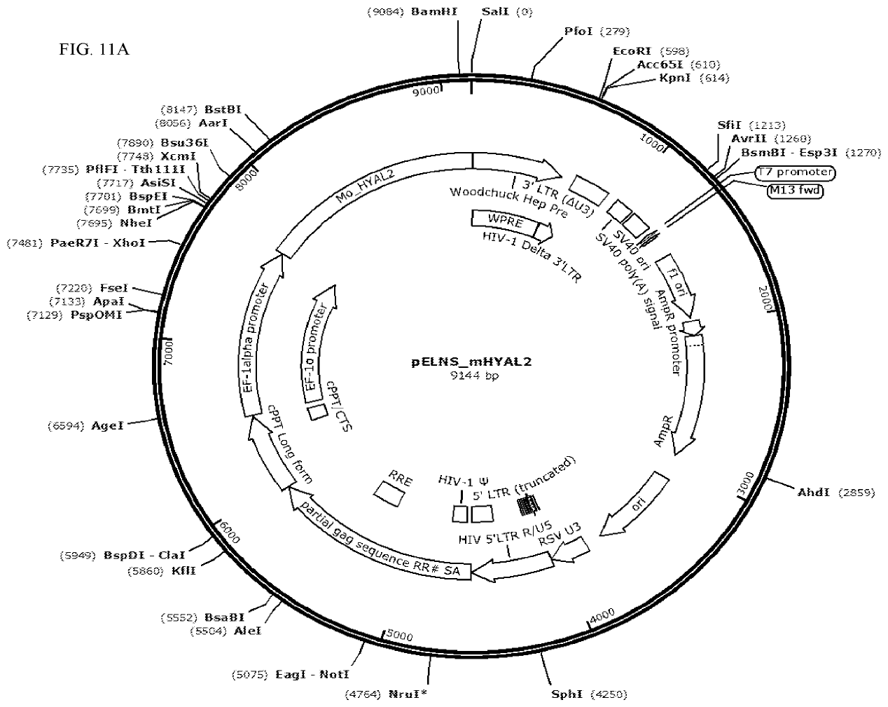

Lentiviral vectors were designed to express hyaluronidase (FIG. 11A) and Spam1 (FIG. 11B) to remodel the extracellular matrix in cancer and disease states.

Hyal2 and Spam1 expression by GEMesys was validated by western blot.

These data demonstrate the process of GEMesy generation through lentiviral transduction and confirm expression of cargo proteins.

### Example 12

This example demonstrates that the immune suppression signature in the pre-metastatic niche is seen in human metastasis, and that this signature may be used to mark the metastatic process as well as evaluate response to targeting of immune suppression.

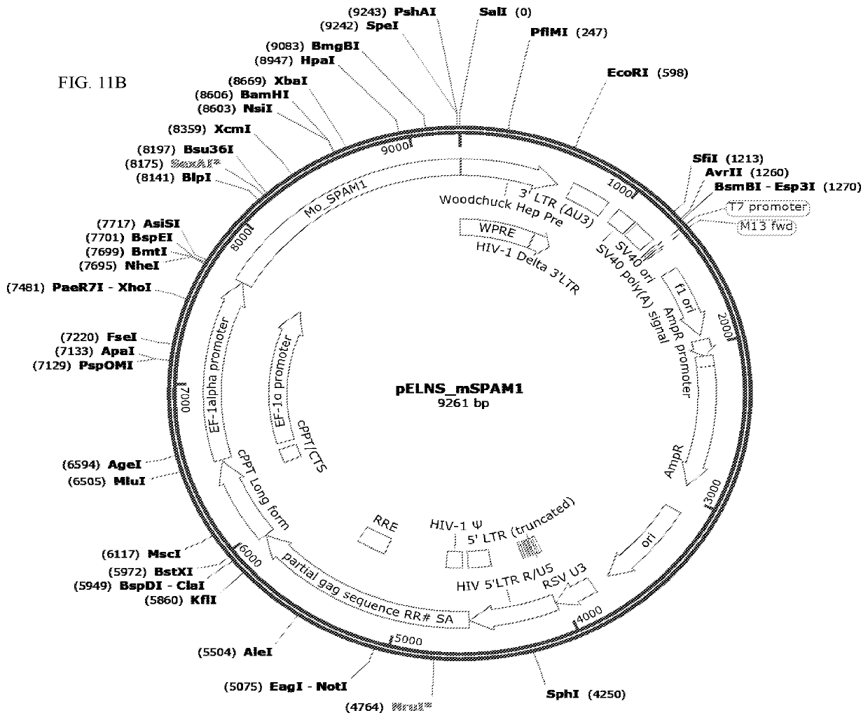

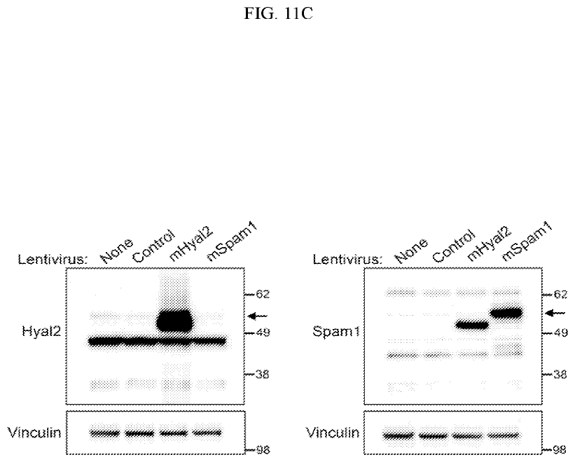

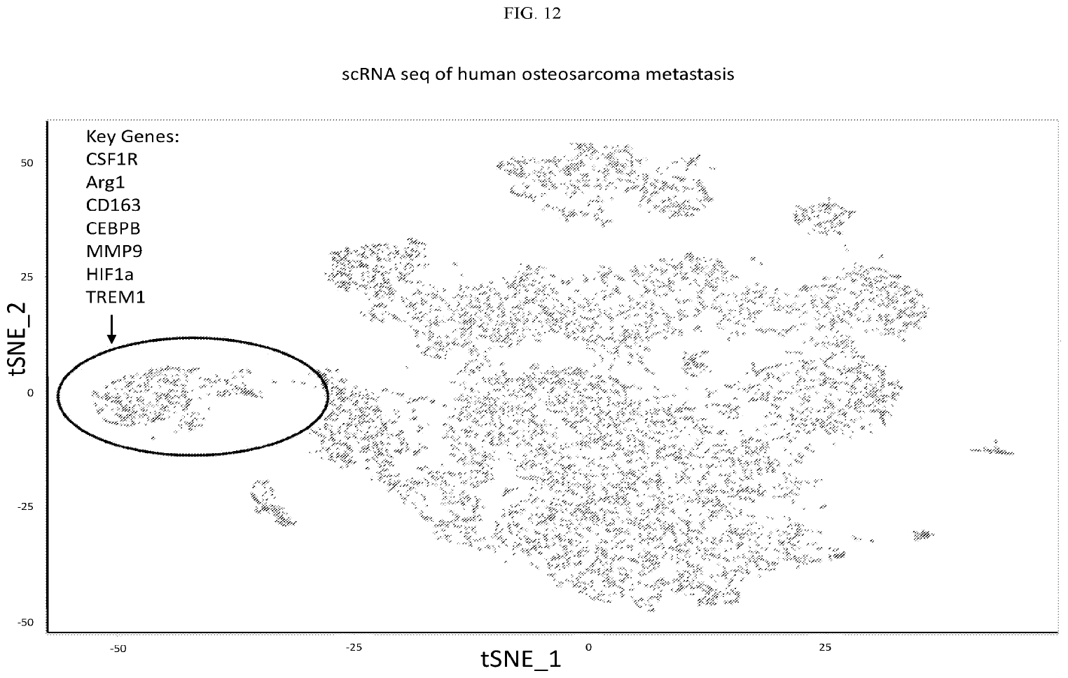

The pre-metastatic niche is a specialized microenvironment composed of activated mesenchymal cells, associated extracellular matrix/matrix remodeling and expansion of hematopoietic stem and progenitor cells that develop into immune suppressive myeloid cells. The immune suppressive microenvironment includes a marked expansion of the myeloid compartment at the expense of the lymphoid compartment over time. The loss of lymphoid cells diminishes the adaptive immune response and provides immune privilege to disseminated tumor cells. The gene signature of the pre-metastatic microenvironment identified key genes involved in immune suppression notably IDO, Arg, TREM, Acod1, MMP9). This signature can be seen in metastatic sarcomas (FIG. 12). Examination of this signature and its reversal in response to IL12 GEMy is depicted in FIG. 4A. Tbx21, IFNγ, Prf1, Ctsw, Klrg1, IL12b, IL12rb1, Lck, Lat, Stat1, Ccl12, Ccl22 constitute a gene signature upregulated in response to IL12 GEMys and may be used during clinical trial evaluation of response to IL12 GEMy therapy.

### Example 13

This example describes an experiment to assess the homing capability of murine and human HSPCs, monocytes, and macrophages.

C57Bl/6 mice are injected orthotopically in the calf muscle with 5×10⁵ M3-9-M cells. Mice are then given 2 mg cyclophosphamide and 5 mg fludarabine IP 48 hrs prior to IV administration of ⁸⁰Zr-oxine labeled IL12-GEMy or vector control GEMy. The cells will be treated with Zr-89 in order to label the cells in the mice and total body imaging at day 0, 2, 4, 8 days after injection.

NSG mice are administered human monocytes derived from the RO fraction of apheresis or CD34+ HSPCs or human macrophages cultured in GMCSF. The cells will be treated with Zr-89 in order to label the cells in the mice and total body imaging will occur at day 0, 2, 4, 8 days after injection. Pilot studies suggest HSPCs home better than macrophages.

### Example 14

This example demonstrates the use of IL12 antibody treatment after administration of IL12 GEMys to inhibit the potential toxicity or to eliminate excessive IL12 signaling.

C57Bl/6 mice are injected orthotopically in the calf muscle with 5×10⁵ M3-9-M cells. Mice are given 8×10⁶IL12 GEMys or vector control GEMys on day 10 after tumor injection. Mice are then given anti-mIL12-p75 or isotype 1 mg/mouse every 5 days for duration of experiment. Mice given anti-mIL12-p75 will show diminution of anti-tumor immune response.

### Example 15

This example demonstrates the use of CRISPR gene editing in myeloid cells to examine and decouple the connection between phagocytosis and immune suppression and functionally alter gene expression to promote or eliminate immune suppression, as well as enhance or diminish phagocytic function in the GEMys in addition to the specific target gene and process.

SC (human monocyte), MD (human monocyte), and THP1 (human acute monocytic leukemia) cell lines are cultured and whole genomic CRISPR/Cas9 guide RNAs are placed in cells and clones selected for screening analysis of impact on downstream assays of immune suppression and phagocytosis. Fluorescent mark of immune suppression or phagocytic function developed for improved screening of particular genes that augment or diminish the process. These screens directly inform genetically engineered myeloid cell design and can be introduced along with additional gene of interest to prevent immune suppression or enhance immune suppressive properties and/or phagocytic function. Arg1 and IDO CRISPR/Cas9 are initial targets.

### Example 16

This example demonstrates the use of genetically engineered myeloid cells expressing TREM2 to ameliorate neurodegeneration and behavioral changes in Alzheimer's disease.

TREM (triggering receptors expressed on myeloid cells) is a cell surface transmembrane glycoprotein. It is expressed on myeloid cells and elevated levels were observed in the pre-metastatic niche and is a key component of the pre-metastatic niche immune suppression gene signature (FIG. 1E). It has also been identified in the myeloid cluster in single cell sequencing performed on osteosarcoma metastasis (FIG. 12).

TREM1 and 2 are expressed on dendritic cells, granulocytes and tissue specific macrophages including osteoclasts, Kuppfer cells and alveolar macrophages. In the brain, microglia exclusively express TREM2. TREM2 expression increases with age and increases in patients with Alzheimer's disease. TREM2 is elevated in brains of mice with Alzheimer's disease, Parkinson's disease and amyotrophic lateral sclerosis, stroke, and traumatic brain injury. Elevated TREM has been confirmed in patients with Alzheimer's disease. TREM2 signals through DAP-12. TREM2 binds LPS or lipoteichoic acids (LTA). Lipids can bind and activate TREMs. ApoE is a major ligand for TREM. TREM1 and 2 modulate myeloid cell number, proliferation and survival.

Alzheimer's disease is a progressive and incurable neurodegenerative disorder characterized by extracellular neuritic plaques and intraneuronal neurofibrillary tangles composed of misfolded and aggregated P amyloid peptide and the microtubule associated protein tau (MAPT or Tau). Early disease is thought to be due to dysfunction in phagocytosis with later changes characterized by neuronal damage and death with pro-inflammatory signaling and hyperactivation of microglia from a homeostatic and tolerogenic phenotype toward a neurodegenerative microglial phenotype characterized by pro-inflammatory cytokines and associated tangles and neuronal loss.

The role of TREM in Alzheimer's disease is not known. The role of TREM in Alzheimer's disease is complex and not clearly understood. Despite the ability of microglia and astrocytes in the clearance of Aβ, the production of pro-inflammatory cytokines like TNFα and IL1β resulting from glial activation are harmful and toxic to neurons. Loss of TREM2 or DPA12 causes Nasu-Hakola disease, a recessive disorder characterized by bone cysts and early dementia. TREM2 has been shown to bind to anionic and zwitterionic lipids found on damaged neurons and AD-associated proteins APOE and Clusterin. The early stages of Alzheimer's disease may have different pathophysiology than later stages. Gliosis may play a neuroprotective role by controlling amyloid load but later become toxic to neurons and catalyst for neurodegeneration. TREM2 can regulate phagocytosis and lysosomal activity in microglia and therefore play a potentially protective role in Alzheimer's disease pathogenesis. TREM2 can also modulate inflammatory signaling. Macrophages lacking TREM2 release higher levels of pro-inflammatory cytokines like TNFα, IL1β, IL6 and NO synthase-2 (NOS2). Elevated TREM2 expression in microglia can have a protective effect.

TREM2 GEMys and TREM2 APOE decoy receptor or APOE TRAP GEMys are administered to aging and disease progression mouse models including APP-PS1 (overexpressing mutated genes for human amyloid precursor protein and presenilin 1) and 5×FAD mice (carrying 5 familial APP and PSEN1 mutations). TREM2 expressing GEMys (TREM2 GEMys) are delivered intravenously or intracerebrally to ameliorate Alzheimer's disease progression. Mice are followed with regular behavioral testing and at endpoint brains will be examined for neuritic plaques and neurofibrillary tangles evidence of neuronal loss and degneration.

APOE is known Alzheimer's disease risk gene and overexpression of APOE in microglia is associated with worsening neurodegeneration. APOE is upregulated in the switch from MO homeostatic and tolerogenic microglia phenotype to a neurodegenerative amoeboid-phagocytic phenotype. This switch is regulated by miR155. Immune suppression and phagocytosis may represent two mutually exclusive or near mutually exclusive programs in myeloid cells. Enhancing APOE sequestering modulates the progression of pro-inflammatory pathways and neuronal loss in Alzheimer's disease or other neurodegenerative conditions.

### Example 17

This example describes experiments wherein CD2AP GEMys that express or are induced to express 6-O-sulfated heparan sulfate proteoglycan as a cell surface anchor are administered to aging and disease progression mouse models of APP-PS1.

Alzheimer's disease is characterized by extracellular deposition of senile plaques, intracellular occurrence of neurofibrillary tangles produced by abnormal aggregation of amyloid beta (AR) and hyperphosphorylation of tau. These plaques and tangles interfere with calcium signaling and synaptic transmission. CD2AP is a scaffolding molecule that regulates the actin cytoskeleton. It is involved in T cell and antigen presenting cell junction. It plays a strong role in dynamic actin remodeling and membrane trafficking during endocytosis and cytokinesis. CD2AP expression in microglia or myeloid cells can enhance synaptic function in neurodegenerative conditions which may ameliorate neuronal loss in Alzheimer's disease. Elevating the expression of CD2AP within the brain could be an effective treatment.

CD2AP GEMys are administered to aging and disease progression mouse models including APP-PS1 (overexpressing mutated genes for human amyloid precursor protein and presenilin 1) and 5×FAD mice (carrying 5 familial APP and PSEN1 mutations). CD2AP GEMys are delivered intravenously or intracerebrally of CD2AP GEMys to ameliorate Alzheimer's disease progression. Mice are followed with regular behavioral testing and at endpoint brains will be examined for neuritic plaques and neurofibrillary tangles and evidence of neuronal loss and degeneration.

### Example 18

This example demonstrates the administration of CD33 DECOY or CD33 TRAP GEMys to aging and disease progression mouse models of APP-PS1.

CD33 is a sialic acid binding immunoglobulin-like lectin that regulates innate immunity but has no known function in the brain. However, CD33 gene has been identified as a risk factor for Alzheimer's disease. CD33 is expressed on microglia. Microglia with CD33 are immunoreactive and correlated with enhanced plaque burden in particular amyloid beta 42 (Aβ42) in AD brain.

Decoy receptor CD33 or TRAP CD33 GEMys are administered to aging and disease progression mouse models including APP-PS1 (overexpressing mutated genes for human amyloid precursor protein and presenilin 1) and 5×FAD mice (carrying 5 familial APP and PSEN1 mutations). Decoy receptor CD33 or TRAP CD33 GEMys are delivered intravenously or intracerebrally to ameliorate Alzheimer's disease progression. Mice are followed with regular behavioral testing and at endpoint brains will be examined for neuritic plaques and neurofibrillary tangles and evidence of neuronal loss and degeneration.

### Example 19

This example demonstrates the use of genetically engineered myeloid cells expressing TREM1 and/or TREM2 TRAP or TREM1/2 Decoy receptors (TREM1/2 TRAP GEMy and TREM1/2 Decoy receptor GEMy). An inflammatory bowel disease (IBD) model is being tested in C57BL/6 mice with DSS-induced colitis. Mice are given TREM1 or TREM2 TRAP or TREM1 or TREM2 decoy receptors with GEMys expressing scFv for intestinal binding for induction of decoy or TRAP expression.

Inflammatory bowel disease refers to chronic inflammatory disorders affecting the gastrointestinal tract. A fine tuning of immune reactivity is essential or chronic inflammation can develop where acute inflammation has not resolved and/or is excessive and leads to tissue damage. The TREM family plays a role in regulation of immune response and can modify pattern recognition receptors. TREM1 mRNA and protein have been shown to be significantly up-regulated in colitis models and the elevation could precede the appearance of histological signs of the disease. Targeting this immune modulatory receptor on myeloid cells will limit or reverse or eliminate the colitis.

### Example 20

This example demonstrates the use of genetically engineered myeloid cells expressing receptors GPR32 GEMys for IBD proresolving receptors that promote specialized pro-resolving mediators (SPMs) being tested in C57/B16 mice with DSS-induced colitis. Mice are given proresolving receptors ChemR23 GEMy, ERV GEMy, FPR2 GEMy, DRV GEMy, GPR32 GEMy, GPR18 GEMY, GPR37 GEMy, or LGR6 GEMy alone or in different combinations and/or at different times and optionally with a nonsignaling scFv for the CSL receptor for intestinal localization and induction of proresolving receptors.

Inflammation initially starts with redness, swelling, pain. It then results in release of chemokines and cytokines followed by lipid mediators—prostaglandins and leukotrienes. This results in neutrophil migration and LTB4-dependent amplification of neutrophil influx. Apoptotic neutrophils can induce macrophage clearance which results in biosynthesis of specialized pro-resolving mediators that reduce expression of IL6 and IFNγ and inhibit the migration and activation of dendritic cells and further cytokine secretion. In the case of ongoing inflammation with continued apoptotic neutrophils activating dendritic cell antigen presentation and T cell activation as a result of hyperactive pro-inflammatory response or an inefficiency to stimulate resolution of inflammation due to disordered function of mediators or failed response to mediators can be prototypical of chronic inflammatory disorders such as atherosclerosis, diabetes, inflammatory bowel disease and arthritis. Rebalancing the myeloid program toward a resolution phenotype restores the immune balance in these disorders without the need for the traditional immune suppression therapeutic approach.

### Example 21

This example demonstrates the use of genetically engineered myeloid cells that express P2ry2 and/or P2ry6 for therapeutic benefit in inflammatory bowel disease in C57/B16 mice with DSS-induced colitis and reversal of neurodegeneration in APP-PS1 mice with neurodegeneration.

The transcriptional phenotype of myeloid cells can be critical in tuning the local immune response. These cells hold key functions in turning adaptive immune arm on for functional anti-microbial or anti-tumor immunity by key transcription factor mediated gene programs as well as reducing acute inflammation and enhancing wound healing by an alternative transcription facto mediated gene program. Manipulating the gene program by altering the transcriptional phenotype of myeloid cells redirects the immune balance in a localized fashion that can hold therapeutic benefit in different altered microenvironments. P2ry2 and P2ry6 are key homeostatic genes in microglia and myeloid cells. Restoring these pathways elicit a cascade of downstream and secreted mediators that can rebalance the microenvironment. Protein level restoration of P2ry12 in APP-PS1 model improves plaque elimination/restoration.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition comprising (a) genetically modified hematopoietic stem and progenitor cells (HSPCs), (b) genetically modified mesenchymal cells, or (c) both (a) and (b), wherein the cells contain a vector comprising a transgene.

2. The composition of claim 1, wherein the composition comprises the HSPCs and the HSPCs are CD34+.

3. The composition of claim 1, wherein the composition comprises the mesenchymal cells and the mesenchymal cells are mesenchymal stem cells.

4. The composition of claim 1, wherein the vector is a viral vector.

5. The composition of claim 4, wherein the viral vector is a lentiviral vector.

6. (canceled)

7. The composition of claim 1, wherein the transgene encodes hyaluronidase.

8. The composition of claim 1, wherein the transgene encodes a scFv, IgG, bispecific antibody, or trispecific antibody.

9. The composition of claim 1, wherein the vector encodes one or more transgenes selected from IL-12, IL-10, CXCL9, CXCL10, TGFβ, IL-2, SMAD, TREM2, CD2AP, a Herpes Simplex Virus Thymidine Kinase (HSVTK)/Ganciclovir (GCV) suicide gene system, and an inducible Caspase suicide gene system.

10. A method for producing genetically modified hematopoietic stem and progenitor cells (HSPCs) comprising:
transfecting isolated mammalian HSPCs with a vector comprising a transgene,
thereby producing genetically modified HSPCs.

11.-12. (canceled)

13. The method of claim 10, further comprising differentiating the genetically modified HSPCs into myeloid cells, thereby producing genetically engineered myeloid cells.

14. The method of claim 13, wherein the genetically engineered myeloid cells are genetically engineered bone marrow-derived CXCR4+ myeloid cells.

15. A method for producing genetically modified mesenchymal cells comprising:
transfecting isolated mesenchymal cells with a viral vector comprising a transgene,
thereby producing genetically modified mesenchymal cells.

16. (canceled)

17. The method of claim 15, wherein the mesenchymal cells are mesenchymal stem cells and the method further comprises differentiating the genetically modified mesenchymal stem cells into stromal cells, thereby producing genetically engineered stromal cells.

18. The method of claim 17, wherein the genetically engineered stromal cells are activated pericytes, myofibroblasts, vascular smooth muscle cells, or combinations thereof.

19.-24. (canceled)

25. A method of treating cancer in a mammal with cancer comprising administering the composition of claim 1 to the mammal.

26.-28. (canceled)

29. A method of reducing tumor growth or reducing or preventing recurrence of tumor in a mammal with cancer comprising administering the composition of claim 1 to the mammal.

30.-31. (canceled)

32. A method of extending survival time of a mammal with cancer comprising administering the composition of claim 1 to the mammal.

33.-34. (canceled)

35. A method of preventing tumor dormancy in a mammal with cancer comprising administering the composition of claim 1 to the mammal.

36.-38. (canceled)

39. A method of reducing or preventing metastasis in a mammal with cancer comprising administering the composition of claim 1 to the mammal.

40.-43. (canceled)

44. A method of treating a neurodegenerative condition, autoimmune disorder, or inflammatory disorder in a mammal comprising administering the composition of claim 1 to the mammal.

45.-50. (canceled)
